SEQUENCE LISTING (2 sequences)

SEQ ID NO: 1 - DNA, 21 bp, Artificial
  Synthetic Primers
aagcttatggatggatggaga

SEQ ID NO: 2 - DNA, 21 bp, Artificial
  Synthetic Primers
ggatcctcagcgggccaaggc

(19) United States
(12) Patent Application Publication
(10) Pub. No.: US 2009/0105251 A1
(43) Pub. Date: Apr. 23, 2009

(54) Renin inhibitors

(76) Inventors: Benjamin Jones, San Diego, CA (US); Andrey A. Kiryanov, San Diego, CA (US); Stephen W. Kaldor, San Diego, CA (US)

Correspondence Address: TAKEDA SAN DIEGO, INC., 10410 SCIENCE CENTER DRIVE, SAN DIEGO, CA, 92121, US

(21) Appl. No.: 11/953,793

(22) Filed: Dec. 10, 2007

Related U.S. Application Data
(60) Provisional application No. 60/886,609, filed on Jan. 25, 2007.

(51) Int. Cl. A61K 31/5377; C07D 409/06; A61K 31/496; A61K 31/495; A61P 35/00; A61P 9/12; C12N 9/99; C07D 241/04; C07D 413/14

(52) U.S. Cl. 514/235.8; 544/379; 514/252.13; 514/255.01; 544/391; 544/121; 435/184

(57) ABSTRACT

Compounds, pharmaceutical compositions, kits and methods are provided for use with renin that comprise a compound selected from the group consisting of:

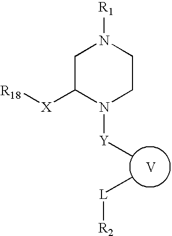

wherein the variables are as defined herein.

### RELATED APPLICATION

This application claims the benefit of U.S. Provisional Application No. 60/886,609, filed Jan. 25, 2007; the disclosure of which is incorporated herein by reference.

### FIELD OF THE INVENTION

The present invention relates to compounds that may be used to inhibit renin, as well as compositions of matter and kits comprising these compounds. The invention also relates to methods for inhibiting renin and treatment methods using compounds according to the present invention.

### BACKGROUND OF THE INVENTION

The renin-angiotensin-aldosterone system (“RAAS”) is one of the hormonal mechanisms involved in regulating pressure/volume homeostasis and also in the development of hypertension, a condition that can progress to more serious cardiovascular diseases such as congestive heart failure. Activation of RAAS begins with secretion of the enzyme renin from juxtaglomerular cells in the kidney.

Renin, a member of the aspartyl protease family, passes from the kidneys into the blood where it cleaves angiotensinogen to generate the decapeptide angiotensin I. Angiotensin I is then cleaved in the lungs, the kidneys and other organs by the angiotensin-converting enzyme (ACE) to form the octapeptide angiotensin II. Angiotensin TI, which is known to work on at least two receptor subtypes (AT₁ and AT₂), increases blood pressure both directly by arterial vasoconstriction and indirectly by liberating from the adrenal glands the sodium-ion-retaining hormone aldosterone. Angiotensin II also produces other physiological effects such as promoting sodium and fluid retention, inhibiting renin secretion, increasing sympathetic nervous system activity, stimulating vasopressin secretion, causing a positive cardiac inotropic effect and modulating other hormonal systems.

Modulation of the RAAS represents a major advance in the treatment of cardiovascular diseases. In particular, the rationale to develop renin inhibitors lies in its specificity (Kleinert H. D., Cardiovasc. Drugs, 1995, 9, 645). The only substrate known for renin is angiotensinogen, which can only be processed (under physiological conditions) by Renin. Inhibitors of the enzymatic activity of renin are therefore expected to bring about a reduction in the formation of angiotensin I and angiotensin II.

In view of the foregoing, renin is an attractive target for the discovery of new therapeutics for cardiovascular disease, hypertension, congestive heart failure, myocardial infarction, renal protection, inflammation, neurological diseases, cancer and other diseases. Accordingly, there is a need to find new renin inhibitors for use as therapeutic agents to treat human diseases.

### SUMMARY OF THE INVENTION

The present invention, in one aspect, relates to compounds that have activity for inhibiting Renin. The present invention also relates to pharmaceutical compositions, articles of manufacture and kits comprising these compounds.

In another aspect, the invention relates to pharmaceutical compositions that comprises a renin inhibitor according to the present invention as an active ingredient. Pharmaceutical compositions according to the invention may optionally comprise 0.001%-100% of one or more inhibitors of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

The invention is also directed to kits and other articles of manufacture for treating disease states associated with Renin.

In one embodiment, a kit is provided that comprises a composition comprising at least one renin inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one renin inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

In another aspect, the invention is related to methods for preparing the compounds, compositions and kits according to the present invention. For example, several synthetic schemes are provided herein for synthesizing compounds according to the present invention.

In another aspect, the invention is related to reagents that may be used in the preparation of the compounds according to the invention.

In another aspect, the invention is related to methods for using compounds, compositions, kits and articles of manufacture according to the present invention. In one embodiment, the compounds, compositions, kits and articles of manufacture are used to inhibit Renin.

In another embodiment, the compounds, compositions, kits and articles of manufacture are used to treat a disease state for which renin possess activity that contributes to the pathology and/or symptomology of the disease state. In another embodiment, a compound is administered to a subject wherein renin activity within the subject is altered, preferably reduced.

In another embodiment, a prodrug of a compound is administered to a subject that is converted to the compound in vivo where it inhibits Renin.

In another embodiment, a method of inhibiting renin is provided that comprises contacting a renin with a compound according to the present invention.

In another embodiment, a method of inhibiting renin is provided that comprises causing a compound according to the present invention to be present in a subject in order to inhibit renin in vivo.

In another embodiment, a method of inhibiting a renin is provided that comprises administering a first compound to a subject that is converted in vivo to a second compound wherein the second compound inhibits renin in vivo. It is noted that the compounds of the present invention may be the first or second compounds.

In another embodiment, a therapeutic method is provided that comprises administering a compound according to the present invention.

In another embodiment, a method of treating a condition in a patient that is known to be mediated by Renin, or which is known to be treated by renin inhibitors, comprising administering to the patient a therapeutically effective amount of a compound according to the present invention.

In another embodiment, a method is provided for treating a disease state for which renin possess activity that contributes to the pathology and/or symptomology of the disease state, the method comprising: causing a compound according to the present invention to be present in a subject in a therapeutically effective amount for the disease state.

In another embodiment, a method is provided for treating a disease state for which renin possess activity that contributes to the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which renin possess activity that contributes to the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by Renin, or that is known to be treated by renin inhibitors.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound of or having the formula” is intended to encompass the compound and all pharmaceutically acceptable ionized forms and solvates, all possible stereoisomers, all possible resonance forms and tautomers, all pharmaceutically acceptable salts and their polymorphs, unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibit renin and thus may themselves be prodrugs for another compound. Such prodrugs of another compound may or may not themselves independently have renin inhibitory activity.

### BRIEF DESCRIPTION OF THE FIGURES

FIG. 1 illustrates SEQ ID NO: 1 and SEQ ID NO: 2 referred to in this application.

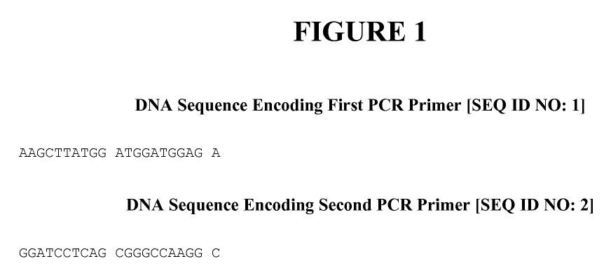

### DEFINITIONS

Unless otherwise stated, the following terms used in the specification and claims shall have the following meanings for the purposes of this Application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃₋₈ rings such as cyclopropane, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkoxy” means the radical —O-alkyl; the alkyl group is as defined in this and can be optionally substituted.

“Alkyl” represented by itself means a straight or branched, saturated or unsaturated, aliphatic radical having a chain of carbon atoms, optionally with oxygen (See “oxaalkyl”) or nitrogen atoms (See “azaalkyl”) between the carbon atoms. C_{X} alkyl and C_{X—Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, pentyl, hexyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenylethyl, 1-phenylethyl, 3-phenylpropyl, 2-thienylmethyl, 2-pyridinylmethyl and the like).

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X—Y} alkylene are typically used where X and Y indicate the number of carbon atoms in the chain. For example, C₁₋₆ alkylene includes methylene (—CH₂—), ethylene (—CH₂CH₂—), trimethylene (—CH₂CH₂CH₂—), tetramethylene (—CH₂CH₂CH₂CH₂—), 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkenylene” means a straight or branched, divalent carbon chain having one or more carbon-carbon double bonds. Examples of alkenylene include ethene-1,2-diyl, propene-1,3-diyl, methylene-1,1-diyl, and the like.

“Alkynylene” means a straight or branched, divalent carbon chain having one or more carbon-carbon triple bonds. Examples of alkynylene include ethyne-1,2-diyl, propyne-1,3-diyl, and the like.

“Alkylidene” means a straight or branched saturated or unsaturated, aliphatic radical connected to the parent molecule by a double bond. C_{X} alkylidene and C_{X—Y} alkylidene are typically used where X and Y indicate the number of carbon atoms in the chain. For example, C₁₋₆ alkylidene includes methylene (═CH₂), ethylidene (═CHCH₃), isopropylidene (═C(CH₃)₂), propylidene (═CHCH₂CH₃), allylidene (═CH—CH═CH₂), and the like).

“Amino” means the radical —NRₐR_{b}, where Rₐ and R_{b} are each independently hydrogen or a non-hydrogen substituent. Representative amino groups include, without limits, —NH₂, —NHCH₃, —N(CH₃)₂, —NHC₁₋₁₀-alkyl, —N(C₁₋₁₀-alkyl)₂, —NHaryl, —NHheteroaryl, —N(aryl)₂, —N(heteroaryl)₂, and the like. Optionally, Rₐ and R_{b} together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (see Heteroaryl).

“Aryl” means a monocyclic or polycyclic ring assembly where all the ring atoms are carbon atoms, and at least one of the rings comprising the ring assembly is an aromatic ring. If one or more ring atoms is not carbon (e.g., N, S), the ring assembly is a heteroaryl. C_{X} aryl and C_{X—Y} aryl are typically used where X and Y indicate the number of carbon atoms in the ring.

“Azaalkyl” means an alkyl, as defined above, except where one or more substituted or unsubstituted nitrogen atoms (—N—) are positioned between carbon atoms of the alkyl. For example, an (C₂₋₆) azaalkyl refers to a chain comprising between 2 and 6 carbons and one or more nitrogen atoms positioned between the carbon atoms.

“Bicyclic” means a two-ringed ring assembly where the two rings are fused together, linked by a single bond or linked by two bridging atoms.

“Bicycloalkyl” means a saturated or partially unsaturated fused bicyclic or bridged polycyclic ring assembly.

“Bicycloaryl” means a ring assembly of two rings, wherein the rings are linked by a single bond or fused and at least one of the rings comprising the ring assembly is an aromatic ring. C_{X} bicycloaryl and C_{X—Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRₐR_{b} where Rₐ and R_{b} are each independently hydrogen or a non-hydrogen substituent.

“Carbocycle” means a ring consisting of carbon atoms.

“Carbocyclic ketone derivative” means a carbocyclic derivative wherein the ring contains a —C(═O)— moiety.

“Carbonyl” typically means a divalent radical —C(═O)—. It is noted that the term “carbonyl” when referring to a monovalent substituent can alternatively refer to a substituted carbonyl or acyl group, —C(═O)Rₐ, where Rₐ is hydrogen or a non-hydrogen substituent on the carbonyl carbon, forming different carbonyl-containing groups including acids, acid halides, aldehydes, amides, esters, and ketones.

“Carboxy” typically means a divalent radical —C(O)O—. It is noted that the term “carboxy” when referring to a monovalent substituent means a substituted carboxy, —C(O)ORₐ, where Rₐ is hydrogen or a non-hydrogen substituent on the carboxyl group forming different carboxy containing groups including acids and esters. It is further noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a radical comprising a non-aromatic, saturated or partially unsaturated, monocyclic, fused or bridged polycyclic ring assembly. C_{X} cycloalkyl and C_{X—Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent radical comprising a saturated or partially unsaturated, monocyclic or polycyclic ring assembly. C_{X} cycloalkylene and C_{X—Y} cycloalkylene are typically used where X and Y indicate the number of carbon atoms in the ring assembly.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a multi-ring assembly wherein the rings comprising the ring assembly are so linked that the ring atoms that are common to two rings are directly bound to each other. The fused ring assemblies may be saturated, partially saturated, carbocyclics, heterocyclics, aromatics, heteroaromatics, and the like. Non-exclusive examples of common fused rings include decalin, naphthalene, anthracene, phenanthrene, indole, benzofuran, purine, quinoline, and the like.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g., halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroalkyl” means alkyl, as defined in this Application, provided that one or more of the atoms within the alkyl chain is a heteroatom.

“Heteroaryl” means a monocyclic or polycyclic ring assembly wherein at least one ring atom is a heteroatom and the remaining ring atoms are carbon, and at least one of the rings comprising the ring assembly is an aromatic ring. Monocyclic heteroaryl groups include, but are not limited to, cyclic aromatic groups having five or six ring atoms, wherein at least one ring atom is a heteroatom and the remaining ring atoms are carbon. The nitrogen atoms of such heteroaryl rings can be optionally quaternerized and the sulfur atoms of such heteroaryl rings can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes polycyclic ring assemblies, wherein a heteroaromatic ring is fused or linked by a bond to one or more rings independently selected from the group consisting of an aromatic ring, a cycloalkyl ring, a cycloalkenyl ring, a heterocycloalkyl ring and another heteroaromatic ring. Bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The polycyclic heteroaryl ring assembly can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined in this Application, provided that one or more of the atoms within the ring assembly is a heteroatom. For example, hetero(C₄₋₁₂)bicycloaryl as used in this Application includes, but is not limited to, indoline, 2-amino-4-oxo-3,4-dihydropteridin-6-yl, tetrahydroisoquinolinyl, and the like.

“Heterocycloalkyl” means cycloalkyl, as defined in this Application, provided that one or more of the atoms forming the ring is a heteroatom. Non-exclusive examples of heterocycloalkyl include piperidyl, 4-morpholyl, 4-piperazinyl, pyrrolidinyl, perhydropyrrolizinyl, 1,3-dioxanyl, 1,4-dioxanyl and the like.

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —NR—, —N⁺(O⁻)═, —O—, —S— or —S(O)₂—, wherein R is hydrogen or a non-hydrogen substituent.

“Heterobicycloalkyl” means bicycloalkyl, as defined in this Application, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like.

“Heterocycle” refers to a ring moiety, saturated, unsaturated or aromatic, where at least one ring atom is a heteroatom and the remaining ring atoms are carbon.

“Heterocycloalkylene” means cycloalkylene, as defined in this Application, provided that one or more of the ring member carbon atoms is replaced by a heteroatom.

“Hydroxy” means the radical —OH.

“IC₅₀” refers to the molar concentration of an inhibitor that produces 50% inhibition of the target enzyme.

“Iminoketone derivative” means a derivative comprising the moiety —C(═NR)—, wherein R is hydrogen or a non-hydrogen substituent attached to the nitrogen.

“Isomers” mean any compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four different substituents (where no two are the same) is termed a “chiral center.” A compound with one chiral center has two enantiomeric forms of opposite chirality. A mixture of equal amounts of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Moiety” means an interconnected group of atoms, generally referred to by its most characteristic structural component. For example, a “carbonyl moiety” refers to groups that contain a carbonyl group.

“Nitro” means the radical —NO₂-“Oxaalkyl” means an alkyl, as defined above, except where one or more oxygen atoms (—O—) are positioned between carbon atoms of the alkyl. For example, an (C₂₋₆)oxaalkyl refers to a chain comprising between 2 and 6 carbons wherein one or more oxygen atoms is positioned between two carbon atoms.

“Oxy” typically means the radical —O—. It is noted that the term “oxy” when referring to a monovalent radical can alternatively refer to a substituents oxy group, —OR—, where R is hydrogen or a non-hydrogen substituent on the oxy radical forming oxy-containing groups including hydroxy, alkoxy, aryloxy, heteroaryloxy and carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into an inhibitor according to the present invention. The prodrug itself may or may not also have renin inhibitory activity. For example, an inhibitor comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, an inhibitor comprising an amine group may be administered as an amide or as an N-alkyl (particularly N-methyl or N-ethyl) that is converted by hydrolysis or oxidation in vivo to the amine compound.

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, β-methoxyethoxymethyl ether, p-methoxybenzyl, methylthiomethyl ether, pivaloyl, silyl ether, carbobenzyloxy, benzyl, tert-butoxycarbonyl, p-methoxyphenyl, 9-fluorenylmethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituent convertible to hydrogen in vivo” means any group that is convertible to a hydrogen atom by enzymological or chemical means including, but not limited to, hydrolysis, reduction and oxidation. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [—(O)CO—C(CH₃)₃], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; —C(O)CH(NH2)CH₃), Arg (arginine), Asn (asparagine), Asp (aspartic acid), Cys (cysteine), Glu (glutamic acid), His (histidine), Ile (isoleucine), Leu (leucine; —C(O)CH(NH₂)CH₂CH(CH₃)₂ Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Orn (ornithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [—(O)CO—C(CH₃)₃], and the like. Suitable peptide residues include peptide residues comprising two to five, and optionally two to three, of the aforesaid amino acid residues. Examples of such peptide residues include, but are not limited to, residues of such peptides as Ala-Ala [—C(O)CH(NH)CH₃—C(O)CH(NH₂)CH₃)], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [—(O)CO—C(CH₃)₃], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl). Further examples of substituents “convertible to hydrogen in vivo” include enzymatic oxidizable groups such as N-alkyls, particularly N-methyl and N-ethyl.

“Substituted or unsubstituted” or “optionally substituted” means that a given moiety may consist of only hydrogen atoms bound at available valences (unsubstituted) or may further comprise one or more non-hydrogen atoms bound through available valencies (substituted). The substituents of an “optionally substituted” group may include, without limitation, one or more substituents independently selected from the group or designated subsets thereof, aldehyde, (C₁₋₁₀)alkyl, alkylene, alkylidene, amide, amino, aminoalkyl, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted.

In one particular embodiment, examples of substituents include, but are not limited to, hydrogen, halo, nitro, cyano, thio, oxy, hydroxy, carbonyloxy, (C₁₋₁₀)alkoxy, (C₄₋₁₄)aryloxy, hetero(C₁₋₃)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —S(O)—. It is noted that the term “sulfinyl” when referring to a monovalent substituent can alternatively refer to a substituted sulfinyl group, —S(═O)₂R, where R is hydrogen or a non-hydrogen substituent on the sulfur atom forming different sulfinyl groups including sulfinic acids, sulfinamides, sulfinyl esters, and sulfoxides.

“Sulfonyl” means the radical —S(O)₂—It is noted that the term “sulfonyl” when referring to a monovalent substituent can alternatively refer to a substituted sulfonyl group, —S(═O)₂R, where R is hydrogen or a non-hydrogen substituent on the sulfur atom forming different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thiocarbonyl” means the radical —C(S)—. It is noted that the term thiocarbonyl when referring to a monovalent substituent can alternatively refer to a substituted thiocarbonyl group, —C(═S)₂R, where R is hydrogen or a non-hydrogen substituent on the carbon atom forming different thiocarbonyl groups including thioacids, thioamides, thioesters, and thioketones.

“Treatment” or “treating” means any administration of a compound of the present invention and includes:

(1) preventing the disease from occurring in an animal which may be predisposed to the disease but does not yet experience or display the pathology or symptomatology of the disease,

(2) inhibiting the disease in an animal that is experiencing or displaying the pathology or symptomatology of the diseased (i.e., arresting further development of the pathology and/or symptomatology), or

(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c}, where Rₐ, R_{b}, and R_{c}, may each independently be hydrogen or any other substituent where the atom attached to the carbon is not a hydrogen atom. Hence, —CF₃, —CH₂OH and —CH₂CN, for example, are all C₁ alkyls.

### DETAILED DESCRIPTION OF THE INVENTION

The present invention relates to compounds, compositions, kits and articles of manufacture that may be used to inhibit Renin. The present invention also relates to methods for inhibiting renin and treatment methods using compounds according to the present invention. The present invention further relates to methods for the preparation of the renin inhibitors of the invention, and to compounds that are useful for the preparation of the renin inhibitors of the invention.

It is noted that the compounds of the present invention may also possess inhibitory activity for other aspartyl proteases (e.g., pepsin, gastricsin, napsin, BACE 1 & 2 and cathepsin D and E) and thus may be used to address disease states associated with these other family members. In addition, the compounds of the present invention may be useful as inhibitors of plasmepsins to treat malaria and as inhibitors of Candida albicans secreted aspartyl proteases to treat fungal infections.

In one aspect, the invention directs to renin inhibiting compounds. In one embodiment, the compounds are of the formula:

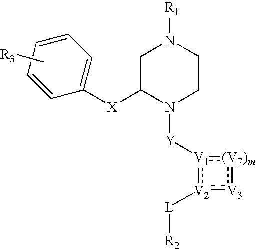

wherein

m is selected from the group consisting of 0, 1, 2, 3, and 4;

L is absent or is a linker providing 1, 2 or 3 atom separation between V₂ and R₂, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

each V₇ is independently selected from the group consisting of, —CR₁₉R₂₀—, —NR₂₁—, —O— and —S—, with the proviso that when m is 2 or 3, no two adjacent V₇ are both substituted with a substituent other than hydrogen;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, the compounds are of the formula:

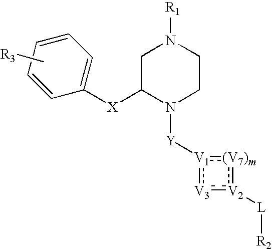

wherein

m is selected from the group consisting of 0, 1, 2, 3, and 4;

L is absent or is a linker providing 1, 2 or 3 atom separation between V₂ and R₂, wherein the atoms of the linker providing the separation are selected from the group consisting of carbon, oxygen, nitrogen, and sulfur atoms;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

V₁ and V₂ are each independently selected from the group consisting of —CR₂₂— and —N—;

V₃ is selected from the group consisting of —CH₂—, —CH═, —NH—, —N═, —O— or —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted,

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, the compounds are of the formula:

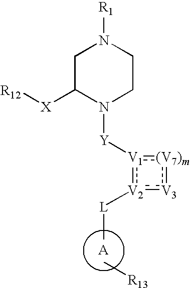

wherein

m is selected from the group consisting of 0, 1, 2, 3, and 4;

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, the compounds are of the formula:

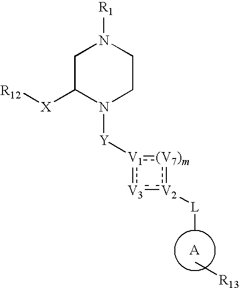

wherein

m is selected from the group consisting of 0, 1, 2, 3, and 4;

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, the compounds are of the formula:

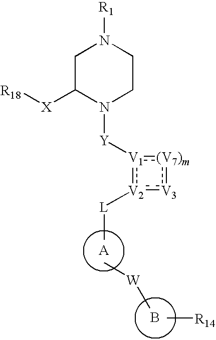

wherein

m is selected from the group consisting of 0, 1, 2, 3, and 4;

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form an oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, the compounds are of the formula:

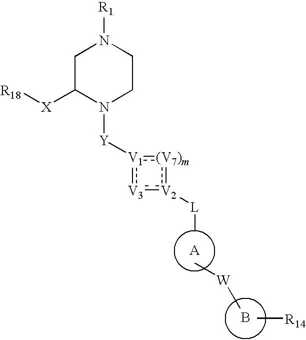

wherein

m is selected from the group consisting of 0, 1, 2, 3, and 4;

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form an oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In yet another embodiment, the compounds are of the formula:

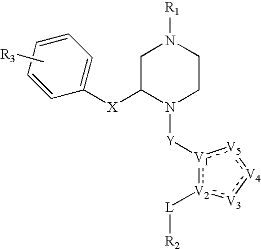

wherein

L is absent or is a linker providing 1, 2 or 3 atom separation between V₂ and R₂, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond,

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In yet another embodiment, the compounds are of the formula:

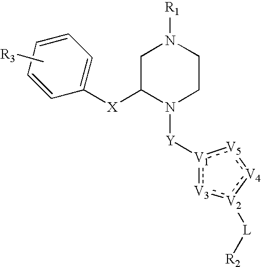

wherein

L is absent or is a linker providing 1, 2 or 3 atom separation between V₂ and R₂, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond,

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, compounds of the present invention are of the formula:

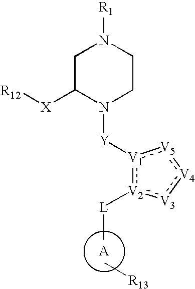

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between ring V and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, compounds of the present invention are of the formula:

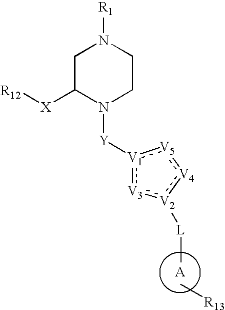

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between ring V and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In a further embodiment, the compounds of the present invention are of the formula:

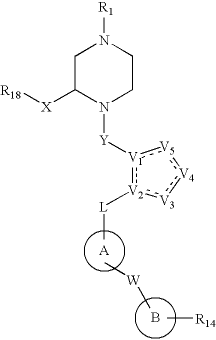

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form an oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In a further embodiment, the compounds of the present invention are of the formula:

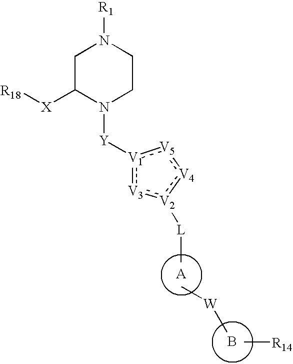

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄ and V₅ are each independently selected from the group consisting of —CR₁₉R₂₀—, —NR₂₁—, —O— and —S—, and at least one of V₄ and V₅ is selected from the group consisting of —CH₂—, —CH═, —NH—, —N═, —O— and —S—;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form an oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In a further embodiment, compounds of the present invention are of the formula:

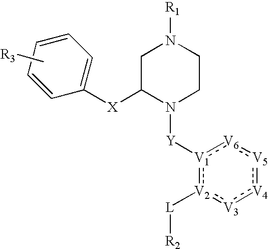

wherein

L is absent or a linker providing 1, 2 or 3 atom separation between V₂ and R₂, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In a further embodiment, compounds of the present invention are of the formula:

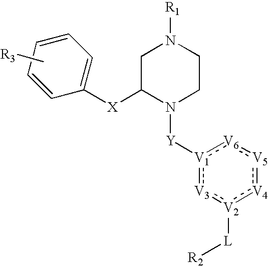

wherein

L is absent or a linker providing 1, 2 or 3 atom separation between V₂ and R₂, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, compounds of the present invention are of the formula:

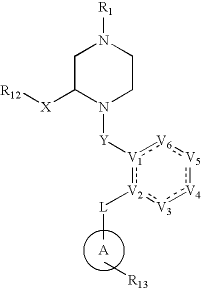

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; wherein the compounds include any polymorph, solvate, ester, tautomer, enantiomer, and pharmaceutical acceptable salt form of the compounds.

In another embodiment, compounds of the present invention are of the formula:

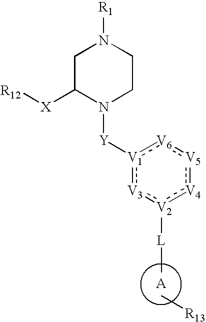

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₂ is a substituted or unsubstituted phenyl or (C₄₋₇)heteroaryl;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; wherein the compounds include any polymorph, solvate, ester, tautomer, enantiomer, and pharmaceutical acceptable salt form of the compounds.

In another embodiment, The compounds of the present invention are of the formula:

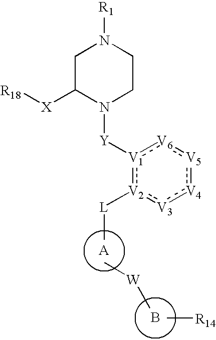

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In another embodiment, The compounds of the present invention are of the formula:

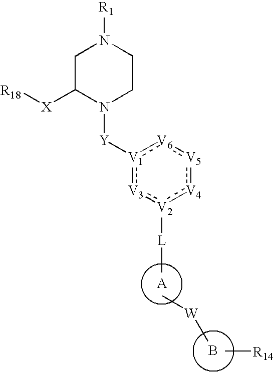

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

L is absent or is a linker providing 1, 2 or 3 atom separation between V₂ and ring A, wherein the atoms of the linker providing the separation are selected from the group consisting of carbon, oxygen, nitrogen, and sulfur atoms;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the compounds include any polymorph, solvate, ester, tautomer, enantiomer, and pharmaceutical acceptable salt form of the compounds.

In one variation of the above embodiments, X is selected from the group consisting of —CH₂— and —CH₂CH₂—. In another variation, X is —CH₂—. In another variation, X is —CH₂CH₂—.

In one variation of the above embodiments and variations, R₄ is selected from the group consisting of hydrogen and halo. In another variation, R₄ is hydrogen.

In one variation of the above embodiments and variations, R₅ is selected from the group consisting of hydrogen and halo. In another variation, R₅ is hydrogen.

In yet another variation of the above embodiments and variations, R₁ is hydrogen. In another variation R₁ is methyl. In another variation R₁ is ethyl.

In still another variation of the above embodiments and variations, R₁₂ is an unsubstituted phenyl. In still another variation of the above embodiments and variations, R₁₂ is a substituted phenyl.

In a further variation of the above embodiments and variations, R₃ is selected from the group consisting of

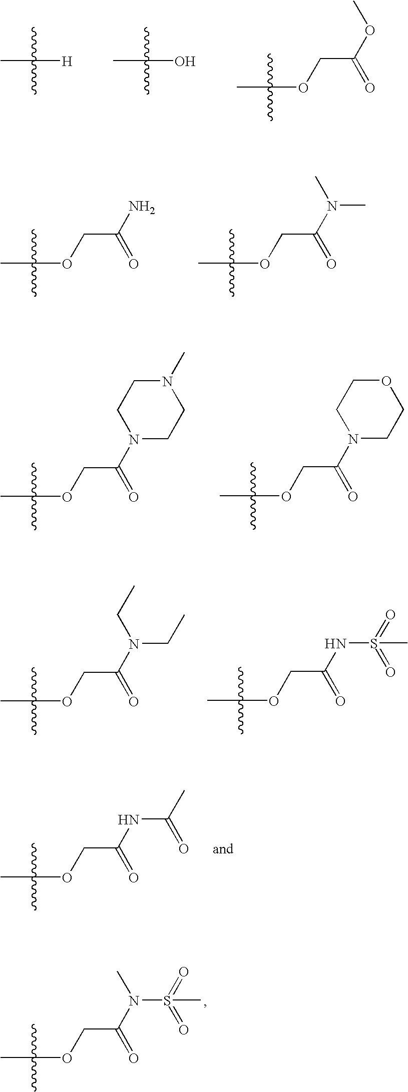

each substituted or unsubstituted.

In another variation of the above embodiments and variations, R₂ is selected from the group consisting of phenyl and hetero(C₁₋₅)aryl, each unsubstituted or substituted with one or more substitutents. In a further variation the substituents of R₂ are each individually selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted. In another further variation, R₂ is substituted with a substituent selected from the group consisting of

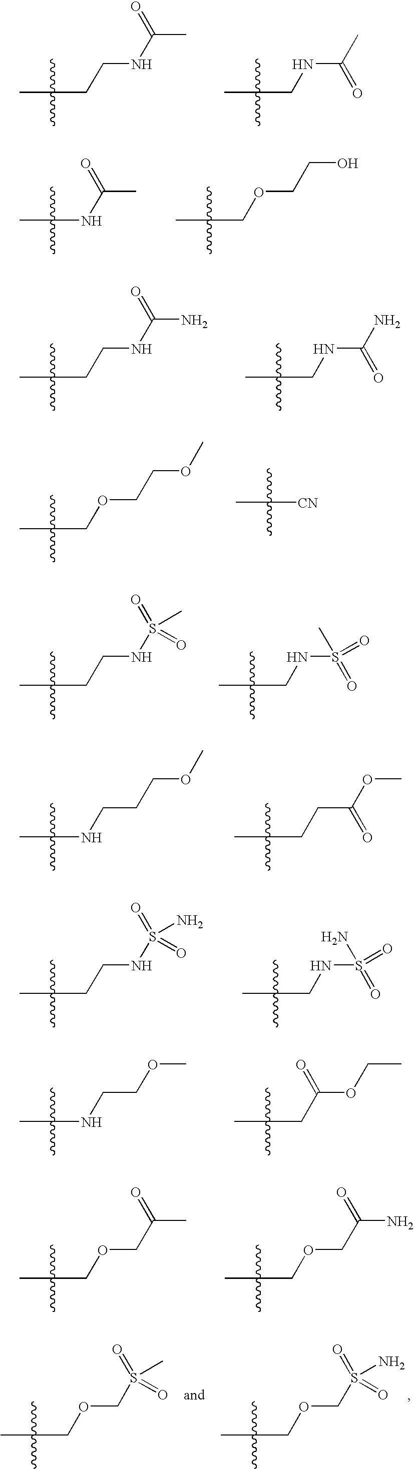

each optionally substituted.

In another variation of the above embodiments and variations, ring A is selected from the group consisting of phenyl and hetero(C₁₋₅)aryl, each substituted or unsubstituted. In another variation, ring A is phenyl.

In another variation of the above embodiments and variations, R₁₃ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, hydroxycarbonyl(C₁₋₁₀)alkyl, alkyl(C₁₋₃)aminocarbonylalkyl(C₁₋₁₀-)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted. In another variation, R₁₃ is selected from the group consisting of

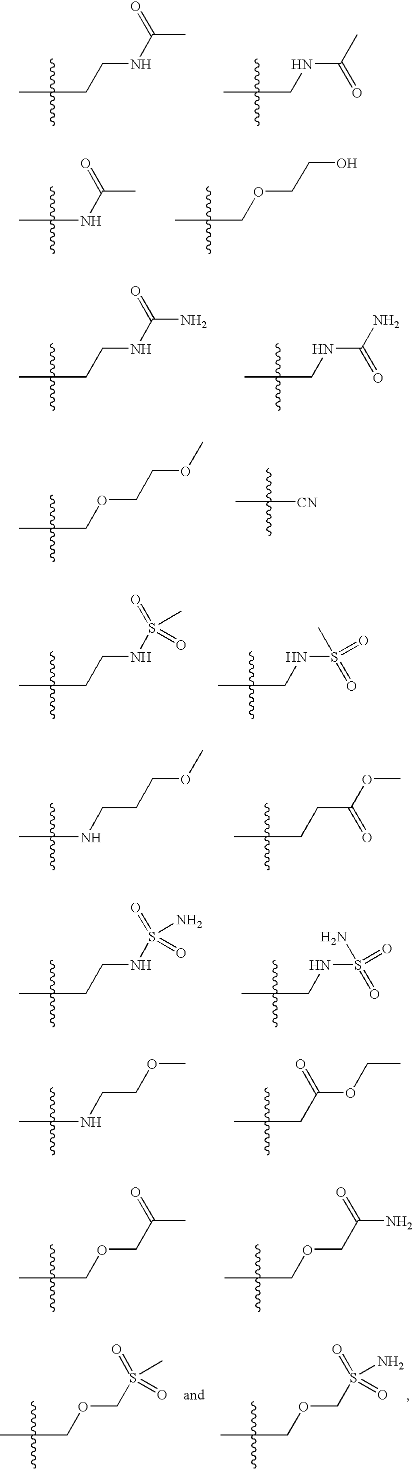

each optionally substituted.

In another variation of the above embodiments and variations, W is selected from the group consisting of —CR₁₅R₁₆—, —NH— and —O—, where R₁₅ and R₁₆ are each independently selected from the group consisting of hydrogen, halo, hydroxyl and substituted or unsubstituted (C₁₋₃)alkyl. In another variation, W is —CH₂—. In another variation, W is —NH—. In a further variation, W is —O—.

In another variation of the above embodiments and variations, R₁₅ is selected from the group consisting of hydrogen, hydroxyl, halo, and substituted or unsubstituted (C₁₋₃)alkyl. In another variation, R₁₅ is hydrogen.

In another variation of the above embodiments and variations, R₁₆ is selected from the group consisting of hydrogen, hydroxyl, halo and substituted or unsubstituted (C₁₋₃)alkyl.

In another variation of the above embodiments and variations, R₁₇ is selected from the group consisting of

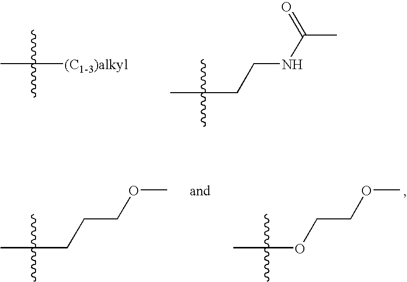

each substituted or unsubstituted.

In another variation of the above embodiments and variations, R₁₈ is selected from the group consisting of (C₁₋₆)alkyl, (C₃₋₇)cycloalkyl, (C₄₋₇)aryl and hetero(C₁₋₅)aryl, each substituted or unsubstituted.

In another variation of the above embodiments and variations, R₁₈ is an unsubstituted phenyl. In yet another variation of the above embodiments and variations, R₁₈ is a unsubstituted phenyl.

In another variation of the above embodiments and variations, ring B is selected from the group consisting of phenyl and hetero(C₁₋₁₀)aryl, each substituted or unsubstituted.

In another variation of the above embodiments and variations, R₁₄ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀-)alkyl, amido(C₁₋₁₀)alkyl, alkoxyalkoxy(C₁₋₁₀)alkyl, alkoxy(C₁₋₁₀)alkyl and alkoxyalkoxy, each substituted or unsubstituted.

In another variation, R₁₄ is selected from the group consisting of

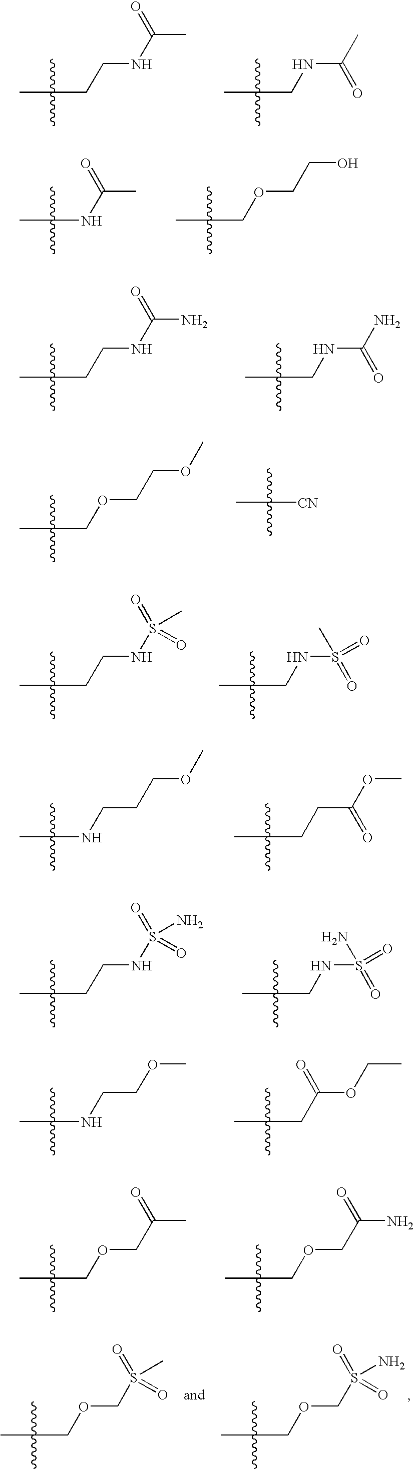

each substituted or unsubstituted.

In another variation of the above embodiments and variations, V₁ is —C═.

In another variation of the above embodiments and variations, V₂ is —C═.

In another variation of the above embodiments and variations, V₃ is selected from the group consisting of —CH═ and —N═.

In another variation of the above embodiments and variations, each V₁, V₂ and V₃ is independently selected from the group consisting of —C═, —N—, —CH═, and —N═.

In another variation of the above embodiments and variations, V₄ is selected from the group consisting of —CH═ and —N═.

In another variation of the above embodiments and variations, V₅ is selected from the group consisting of —CH═ and —N═.

In another variation of the above embodiments and variations, V₆ is selected from the group consisting of —CH═ and —N═.

In another variation of the above embodiments and variations, R₁₉ is selected from the group consisting of hydrogen, halo, oxy, hydroxyl, (C₁₋₃)alkyl and alkoxy, each substituted or unsubstituted.

In another variation of the above embodiments and variations, R₂₀ is selected from the group consisting of hydrogen, halo, oxy, hydroxyl, (C₁₋₃)alkyl and alkoxy, each substituted or unsubstituted.

In another variation of the above embodiments and variations, R₂₁ is selected from the group consisting of hydrogen and substituted or unsubstituted (C₁₋₃)alkyl.

In another variation of the above embodiments and variations, R₂₂ is selected from the group consisting of hydrogen and substituted or unsubstituted (C₁₋₃)alkyl. In another variation,

R₂₂ is hydrogen. In yet another variation, R₂₂ is absent.

In yet another variation of the above embodiments and variations, L is absent.

In yet another variation of the above embodiments and variations, Y is —C(O)—.

Particular examples of compounds according to the present invention include, but are not limited to:
((R)-2-benzylpiperazin-1-yl)(3-(2-phenoxyphenyl)thiophen-2-yl)methanone,
((S)-2-benzylpiperazin-1-yl)(2-phenylcyclopropyl)methanone,
(R)-(2-benzylpiperazin-1-yl)(3′,4′-dimethylbiphenyl-2-yl)methanone,
(R)-(2-benzylpiperazin-1-yl)(biphenyl-2-yl)methanone,
((R)-2-benzylpiperazin-1-yl)(2′-chlorobiphenyl-2-yl)methanone,
(R)-(2-benzylpiperazin-1-yl)(1-phenyl-1H-pyrazol-5-yl)methanone,
(R)-(2-benzylpiperazin-1-yl)(5-phenyloxazol-4-yl)methanone,
((R)-2-benzylpiperazin-1-yl)(1-(2-(trifluoromethyl)phenyl)-1H-imidazol-2-yl)methanone,
2-(2-(2-((R)-2-benzylpiperazine-1-carbonyl)thiophen-3-yl)phenoxy)-4,6-dimethylnicotinonitrile,
2-(2-(2-((R)-2-benzylpiperazine-1-carbonyl)thiophen-3-yl)phenoxy)benzonitrile,
2-(2-(2-((R)-2-benzylpiperazine-1-carbonyl)thiophen-3-yl)phenoxy)nicotinonitrile,
((R)-2-benzylpiperazin-1-yl)(2-(2-phenoxyphenyl)thiophen-3-yl)methanone,
((R)-2-benzylpiperazin-1-yl)(1,3-dimethyl-4-(2-phenoxyphenyl)-1H-pyrazol-5-yl)methanone,
((R)-2-benzylpiperazin-1-yl)(1-ethyl-3-methyl-4-(2-phenoxyphenyl)-1H-pyrazol-5-yl)methanone,
((R)-2-benzylpiperazin-1-yl)(5-fluoro-2′-phenoxybiphenyl-2-yl)methanone,
((R)-2-benzylpiperazin-1-yl)(4-fluoro-2′-phenoxybiphenyl-2-yl)methanone,
((R)-2-benzylpiperazin-1-yl)(1-methyl-4-(2-phenoxyphenyl)-1H-pyrazol-3-yl)methanone,
(R)-(3-(1-benzyl-1H-pyrazol-4-yl)thiophen-2-yl)(2-benzylpiperazin-1-yl)methanone,
((R)-2-benzylpiperazin-1-yl)(2-(biphenyl-2-yl)-thiophen-3-yl)-methanone,
(R)-2-(2-(2-benzylpiperazine-1-carbonyl)phenylthio)benzonitrile,
(R)-(2-benzylphenyl)(2-benzylpiperazin-1-yl)methanone,
(R)-(2-benzoylphenyl)(2-benzylpiperazin-1-yl)methanone,
(R)-(2-(benzyloxy)phenyl)(2-benzylpiperazin-1-yl)methanone,
1-benzhydryl-4-((R)-2-benzylpiperazine-1-carbonyl)pyrrolidin-2-one,
(R)-(2-benzylpiperazin-1-yl)(2-(4-methoxybenzoyl)phenyl)methanone,
((R)-2-benzylpiperazin-1-yl)(1-(naphthalen-1-yl)-1H-imidazol-5-yl)methanone,
((R)-2-benzylpiperazin-1-yl)(1-(biphenyl-2-yl)-1H-imidazol-5-yl)methanone,
((R)-2-benzylpiperazin-1-yl)(2-(3,5-dimethyl-1H-pyrazol-4-yl)phenyl)methanone,
(R)-(2-benzylpiperazin-1-yl)(3-methyl-5-phenylisoxazol-4-yl)methanone,
(R)-(2-benzylpiperazin-1-yl)(5-phenyl-2-(pyridin-3-yl)thiazol-4-yl)methanone,
(R)-(2-benzylpiperazin-1-yl)(4′-fluorobiphenyl-2-yl)methanone,
(R)-(2-(benzo[d]thiazol-2-yl)phenyl)(2-benzylpiperazin-1-yl)methanone,
((R)-2-benzylpiperazin-1-yl)(3-(2-chlorophenyl)-5-cyclopropylisoxazol-4-yl)methanone,
(R)-(6-benzyl-2-methyl-6H-thieno[2,3-b]pyrrol-5-yl)(2-benzylpiperazin-1-yl)methanone,
(R)-(2-benzylpiperazin-1-yl)(2,2-dimethyl-2,3-dihydrobenzofuran-7-yl)methanone,
(R)-(2-benzylpiperazin-1-yl)(4-(4-chlorophenyl)thiophen-2-yl)methanone,
(R)-(2-benzylpiperazin-1-yl)(1-((5-methyl-2-phenyloxazol-4-yl)methyl)-1H-indazol-3-yl)methanone,
(R)-(2-benzylpiperazin-1-yl)(1-tert-butyl-5-phenyl-1H-pyrazol-4-yl)methanone,
N-(2-(2-(2-((R)-2-benzylpiperazine-1-carbonyl)thiophen-3-yl)phenoxy)phenethyl)acetamide,
N-((2-(2-(2-((R)-2-benzylpiperazine-1-carbonyl)thiophen-3-yl)phenoxy)pyridin-3-yl)methyl)acetamide,
((R)-2-benzylpiperazin-1-yl)(2-(2-(morpholinomethyl)phenyl)-thiophen-3-yl)methanone,
((R)-2-benzylpiperazin-1-yl)(1-(2-phenoxyphenyl)-1H-pyrrol-2-yl)methanone,
(R)-(2-benzylpiperazin-1-yl)(4-(2-phenoxyphenyl)thiophen-2-yl)methanone,
(R)-(2-benzylpiperazin-1-yl)(5-(2-phenoxyphenyl)thiophen-2-yl)methanone,
(R)-(2-benzylpiperazin-1-yl)(5-(2-phenoxyphenyl)furan-2-yl)methanone, and
(R)-(2-benzylpiperazin-1-yl)(5-(biphenyl-2-yl)-1,3,4-oxadiazol-2-yl)methanone.

It is noted that the compounds of the present invention may be in the form of a pharmaceutically acceptable salt. It is further note that the compounds of the present invention may be in a mixture of stereoisomers, or the compound may comprise a single stereoisomer.

In another aspect, the present invention is related to a pharmaceutical composition comprising as an active ingredient a compound according to any one of the above embodiments and variations. In one embodiment, the composition is a solid formulation adapted for oral administration. In another embodiment, the composition is a liquid formulation adapted for oral administration. In yet another embodiment, the composition is a tablet. In still another embodiment, the composition is a liquid formulation adapted for parenteral administration.

In another embodiment, the pharmaceutical composition comprises a compound according to any one of the above embodiments and variations, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In another aspect, the invention is related to a kit which comprises a compound of any one of the above embodiments and variations; and instructions which comprise one or more forms of information selected from the group consisting of indicating a disease state for which the composition is to be administered, storage information for the composition, dosing information and instructions regarding how to administer the composition. In one particular variation, the kit comprises the compound in a multiple dose form.

In still another aspect, the invention is related to an article of manufacture comprising a compound of any one of the above embodiments and variations and packaging materials. In one embodiment, the packaging material comprises a container for housing the compound. In another embodiment, the container comprises a label indicating one or more members of the group consisting of a disease state for which the compound is to be administered, storage information, dosing information and/or instructions regarding how to administer the compound. In another embodiment, the article of manufacture comprises the compound in a multiple dose form.

In a further aspect, the invention is related to a therapeutic method comprising administering a compound to a subject.

In one embodiment, the method comprises contacting renin with a compound of any one of the above embodiments and variations.

In yet another embodiment is a method of inhibiting renin which comprises causing a compound of any one of the above embodiments and variations to be present in a subject in order to inhibit renin in vivo.

A further embodiment is a method of inhibiting renin which comprises administering a first compound to a subject that is converted in vivo to a second compound wherein the second compound inhibits renin in vivo, the second compound being a compound according to any one of the above embodiments and variations.

Another further embodiment is a method of treating a disease state for which renin possesses activity contributes to the pathology and/or symptomology of the disease state. In one variation, the method comprises causing a compound of any one of the above embodiments and variations to be present in a subject in a therapeutically effective amount for the disease state. In another variation, the method comprises administering a compound of any one of the above embodiments and variations to a subject, wherein the compound is present in the subject in a therapeutically effective amount for the disease state. In a further variation, the method comprises administering a first compound to a subject that is converted in vivo to a second compound wherein the second compound inhibits renin in vivo, the second compound being a compound according to any one of the above embodiments and variations.

In one variation of the above embodiments and variations, the disease state is selected from the group consisting of cardiovascular disease, hypertension, congestive heart failure, myocardial infarction, renal protection, inflammation, neurological disease and cancer.

In another aspect, the invention is related to methods for the preparation of the compounds of the invention. In one embodiment, the method comprises

coupling a compound of the formula

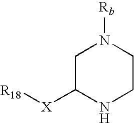

to a compound of the formula R′—Y-J,

under conditions that form a compound of the formula

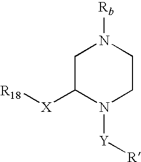

wherein

J is a leaving group;

X is —(CR₄R₅)ₙ—, where n is selected from the group consisting of 1 and 2;

Y is selected from the group consisting of —C(O)— and —S(O)₂—;

R_{b} is selected from the group consisting of R₁, benzyl and Boc;

R′ is a substituted ring;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In another embodiment, R′ is selected from the group consisting of

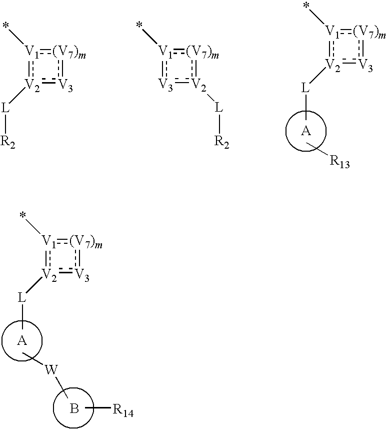

wherein

m is selected from the group consisting of 0, 1, 2, 3, and 4;

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

V₁ and V₂ are each independently selected from the group consisting of —CR₂₂— and —N—;

V₃ is selected from the group consisting of —CH₂—, —CH═, —NH—, —N═, —O— and —S—,

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

L is absent or is a linker providing 1, 2 or 3 atom separation between ring V and R₂ or ring A, wherein the atoms of the linker providing the separation are selected from the group consisting of carbon, oxygen, nitrogen, and sulfur atoms;

W is absent or is selected from the group consisting of —CR₁₅R₁₆—, —NR₁₇—, —O— and —S—;

R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond; and

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring; and

wherein the reaction product includes any hydrate, solvate, tautomer, enantiomer, and pharmaceutically acceptable salt form of the reaction product.

In one variation of the preceding embodiments and variations, Y is —C(O)—.

In another variation, L is absent or is selected from the group consisting of —S—, —C(O)— and —CH₂—. In another variation, L is absent.

In yet another variation, W is absent. In another variation, W is selected from the group consisting of —CR₁₅R₁₆—, —NH— and —O—, and where R₁₅ and R₁₆ are each independently selected from the group consisting of hydrogen, halo, hydroxyl and substituted and unsubstituted (C₁₋₃)alkyl. In yet another variation, W is —NH—. In still another variation, W is —O—. In yet another variation, W is —CH₂—.

In another embodiment, the method of the invention comprises coupling a compound of the formula

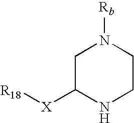

to a compound of the formula

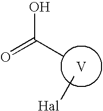

under conditions that form a product of the formula

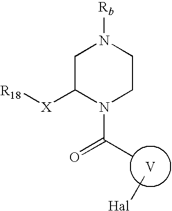

wherein

ring V is a 3, 4, 5, 6, or 7 membered, optionally substituted, saturated, unsaturated, or aromatic carbocycle or heterocycle;

Hal is selected from the group consisting of —Br, —I, —OTf, and —OMs;

X is —(CR₄R₅)ₙ—, where n is selected from the group consisting of 1 and 2;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and

the compound formed includes any hydrate, solvate, tautomer, enantiomer, and pharmaceutically acceptable salt form of the reaction product.

In one variation, the method of the preceding embodiment further comprises

reacting the reaction product of the formula

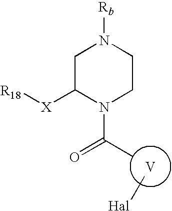

with a boronic acid derivative selected from the group consisting of

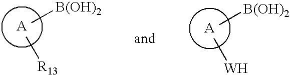

under conditions that form a reaction product selected from the group consisting of

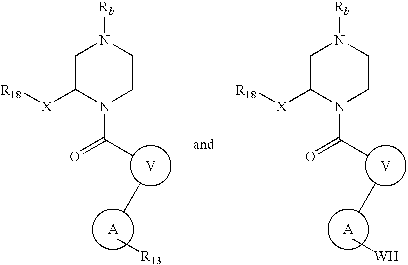

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring V is a 3, 4, 5, 6, or 7 membered, optionally substituted, saturated, unsaturated, or aromatic carbocycle or heterocycle;

Hal is selected from the group consisting of —Br, —I, —OTf, and —OMs;

X is —(CR₄R₅)ₙ—, where n is selected from the group consisting of 1 and 2;

W is absent or is selected from the group consisting of —CR₁₅R₁₆—, —NR₁₇—, —O— and —S—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In another variation of the above embodiments and variations, R₁₃ is selected from the group consisting of

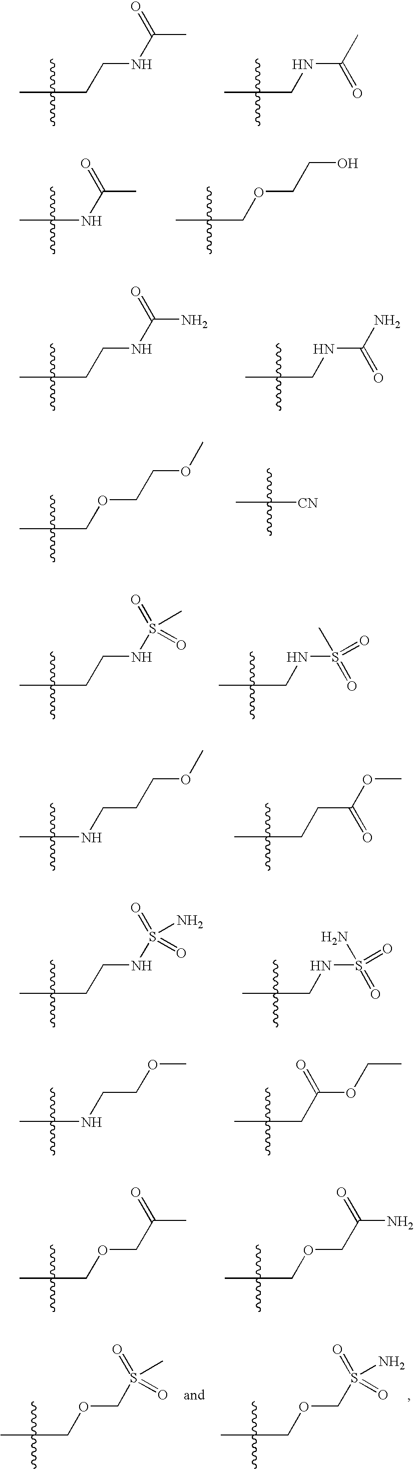

each substituted or unsubstituted.

In another variation of the preceding embodiment, the method further comprises

reacting the compound having the formula

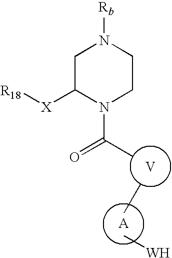

with a cyclic halide of the formula

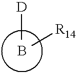

under conditions that form a reaction product of the formula

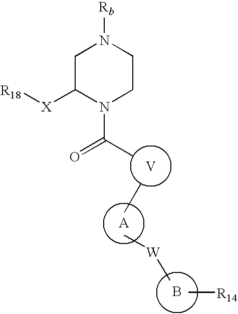

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring V is a 3, 4, 5, 6, or 7 membered, optionally substituted, saturated, unsaturated, or aromatic carbocycle or heterocycle;

D is selected from the group consisting of —F, —Cl and —Br;

X is —(CR₄R₅)ₙ—, where n is selected from the group consisting of 1 and 2;

W is absent or is selected from the group consisting of —CR₁₅R₁₆—, —NR₁₇—, —O— and —S—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and

R₁₄ is selected from the group consisting of

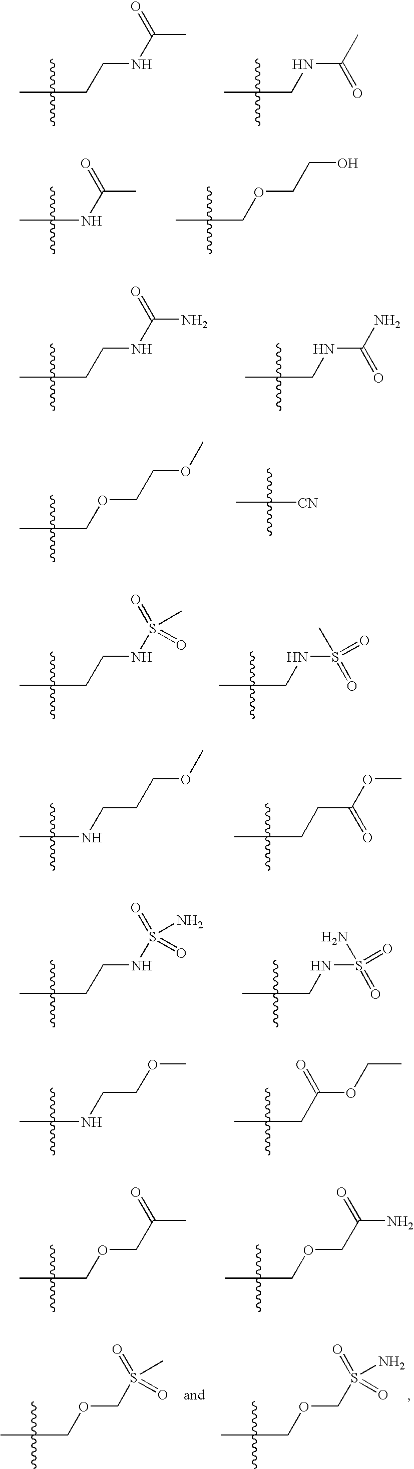

each substituted or unsubstituted.

In another variation of the preceding embodiment where R₁₄ is —CN, the method further comprises converting the —CN group under conditions that forms an acyl-derivative having the formula

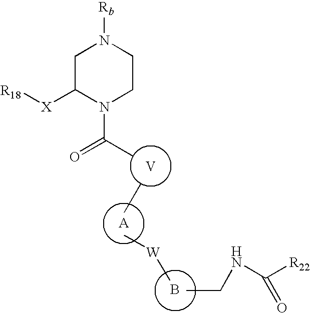

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring V is a 3, 4, 5, 6, or 7 membered, optionally substituted, saturated, unsaturated, or aromatic carbocycle or heterocycle;

X is —(CR₄R₅)ₙ—, where n is selected from the group consisting of 1 and 2;

W is absent or is selected from the group consisting of —CR₁₅R₁₆—, —NR₁₇—, —O— and —S—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and

R₂₂ is selected from the group containing C₁₋₁₀alkyl and amino, substituted or unsubstituted.

In one variation of any one of the above embodiments and variations, the method further comprises

coupling a compound of the formula

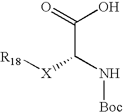

to a compound of the formula

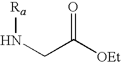

under conditions that form a compound of the formula

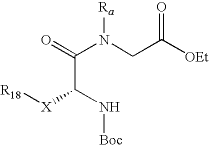

cyclizing the compound immediately above to produce a compound of the formula

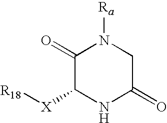

reducing the compound immediately above to produce the compound of the formula

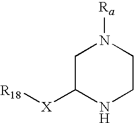

wherein

X is —(CR₄R₅)ₙ—, where n is selected from the group consisting of 1 and 2;

Rₐ is R₁ or benzyl;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In yet another variation, X is selected from the group consisting of —CH₂— and —CH₂CH₂—.

In another variation of any one of the above embodiments and variations, R₁₈ is a substituted phenyl and the substituent is selected from the group consisting of

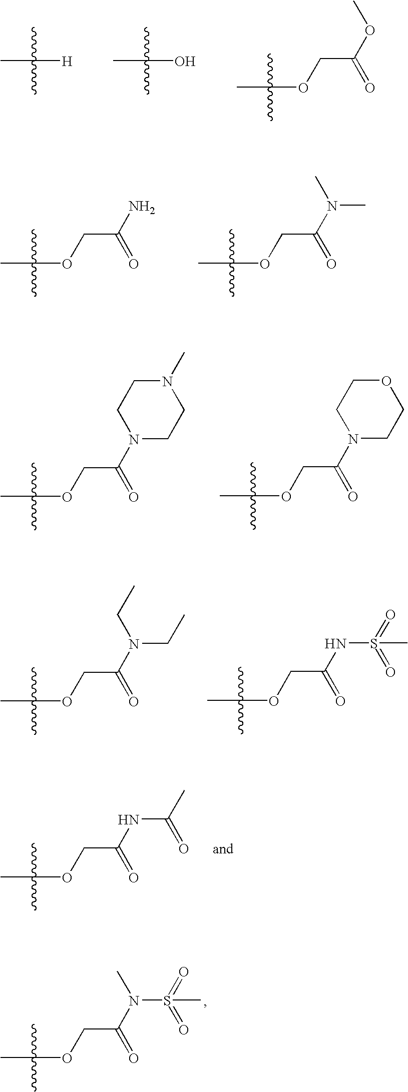

substituted or unsubstituted.

In one variation of any one of the above embodiments and variations, ring V is selected from the group consisting of:

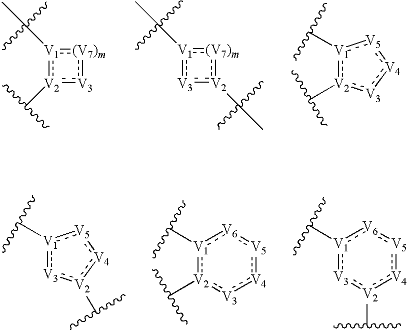

wherein

m is selected from the group consisting of 0, 1, 2, 3, and 4;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring.

In another variation of any one of the above embodiments and variations, ring A is selected from the group consisting of (C₃₋₇)cycloalkyl, hetero(C₃₋₇)cycloalkyl, (C₄₋₇)aryl and hetero(C₁₋₆)aryl, each substituted or unsubstituted. In yet another variation, ring A is selected from the group consisting of phenyl and hetero(C₁₋₅)aryl, each substituted or unsubstituted. In yet another variation, ring A is substituted with a substituent selected from the group consisting of

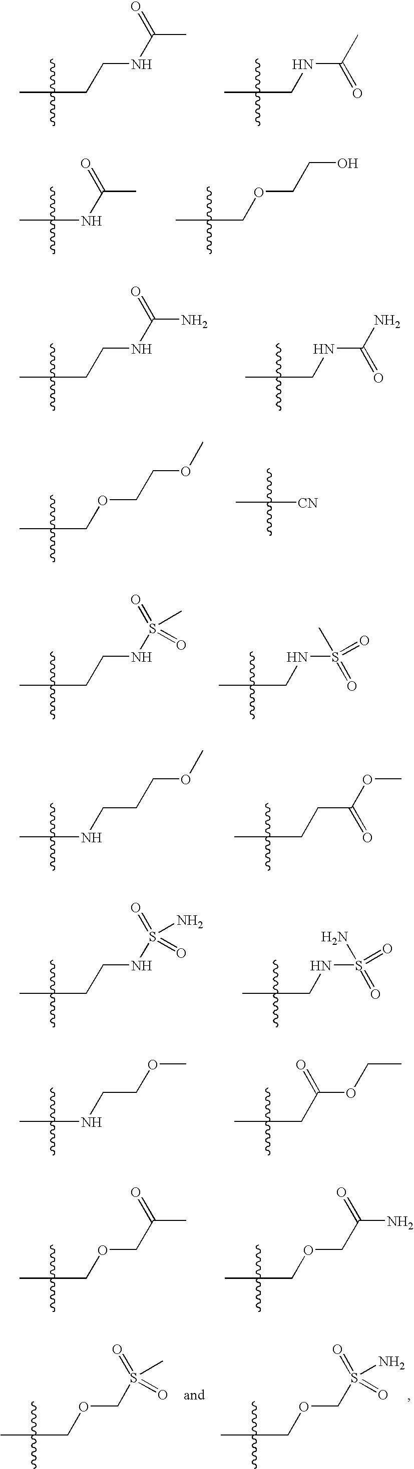

each substituted or unsubstituted.

In another variation of any one of the above embodiments and variations, ring B is selected from the group consisting of phenyl and hetero(C₁₋₁₀)aryl, each substituted or unsubstituted. In another variation, ring B is phenyl. In yet another variation, ring B is substituted with a substituent selected from the group consisting of

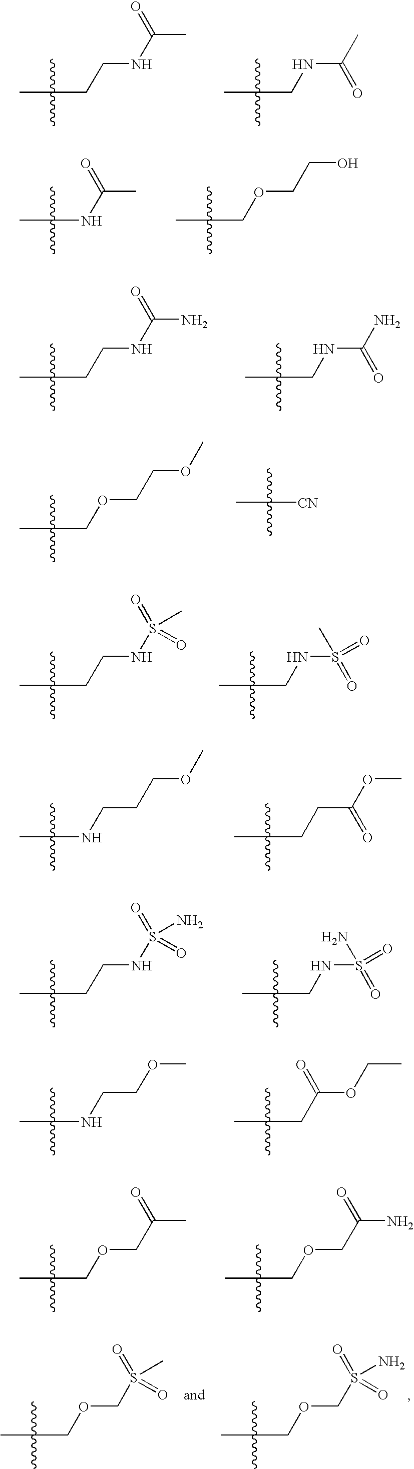

each substituted or unsubstituted.

A further aspect of the invention relates to reagents that may be used to synthesize the compounds of the invention. In one embodiment, the reagents are of the formula

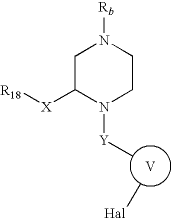

wherein

ring V is a 3, 4, 5, 6, or 7 membered, optionally substituted, saturated, unsaturated or aromatic carbocycle or heterocycle;

Hal is selected from the group consisting of —Br, —I, —OTf, and —OMs;

X is —(CR₄R₅)ₙ—, where n is selected from the group consisting of 1 and 2;

Y is selected from the group consisting of —C(O)— and —S(O)₂—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In another embodiment, the reagents are of the formula

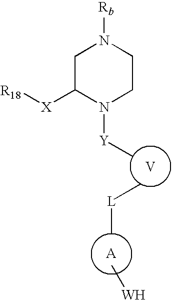

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring V is a 3, 4, 5, 6, or 7 membered, optionally substituted, saturated, unsaturated or aromatic carbocycle or heterocycle;

L is absent or is a linker providing 1, 2 or 3 atom separation between ring V and ring A, wherein the atoms of the linker providing the separation are selected from the group consisting of carbon, oxygen, nitrogen, and sulfur atoms;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In another embodiments, the reagents are of the formula

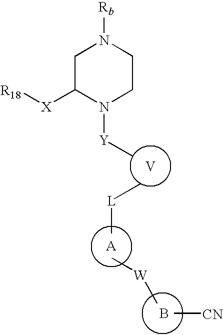

wherein

ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;

ring V is a 3, 4, 5, 6, or 7 membered, optionally substituted, saturated, unsaturated, or aromatic carbocycle or heterocycle;

L is absent or is a linker providing 1, 2 or 3 atom separation between ring V and ring A, wherein the atoms of the linker providing the separation are selected from the group consisting of carbon, oxygen, nitrogen, and sulfur atoms;

W is absent or is selected from the group consisting of —CR₁₅R₁₆—, —NR₁₇—, —O— and —S—;

X is —(CR₄R₅)ₙ—, where n is selected from the group consisting of 1 and 2;

Y is selected from the group consisting of —C(O)— and —S(O)₂—;

R_{b} is selected from the group consisting of R₁, benzyl and a Boc group;

R₁ is selected from the group consisting of hydrogen and a substituent convertible in vivo to hydrogen;

R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;

R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;

R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond; and

R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

In one variation of any one of the above embodiments and variations, ring V is selected from the group consisting of:

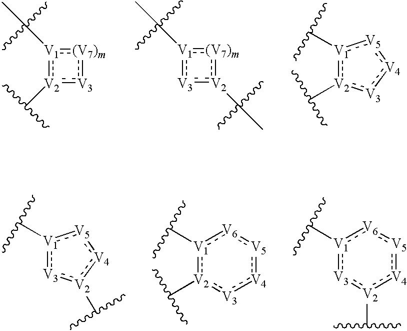

wherein

m is selected from the group consisting of 0, 1, 2, 3, and 4;

V₁ and V₂ are each independently selected from the group consisting of —CR₂₂— and —N—;

V₃ is selected from the group consisting of —CH₂—, —CH═, —NH—, —N═, —O— and —S—;

V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;

each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;

R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and

R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;

or any two R₁₉, R₂₀ and R₂₁ are taken together to form a 3, 4, 5, 6 or 7 membered ring.

In one variation of any one of the preceding embodiments and variations, R₁₈ is a substituted phenyl. In another variation R₁₈ is a substituted phenyl, wherein the substituent is selected from the group consisting of

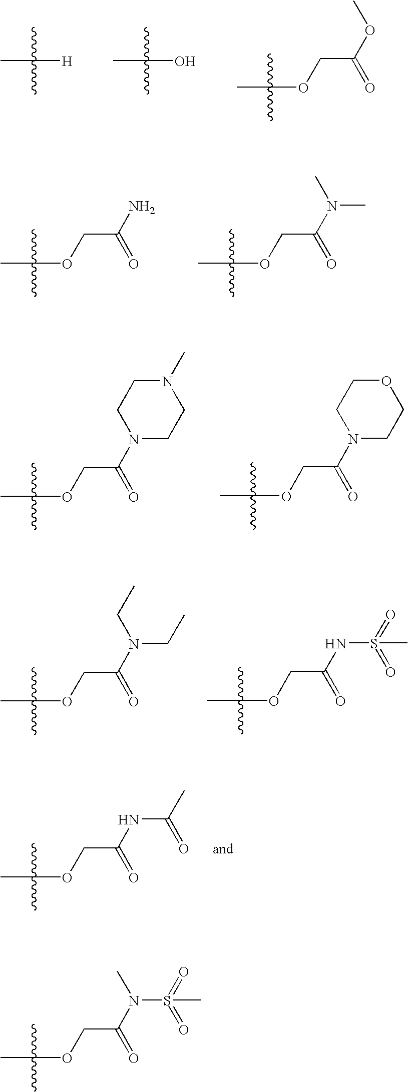

In one variation of any one of the preceding embodiments and variations, L is absent.

In one variation of any one of the preceding embodiments and variations, Y is —C(O)—.

In one variation of any one of the preceding embodiments and variations, R₁ is hydrogen, methyl or ethyl. In another variation, R₁ is hydrogen. In another variation, R₁ is methyl. In another variation, R₁ is ethyl.

### Salts, Hydrates, and Prodrugs of Renin Inhibitors

It should be recognized that the compounds of the present invention may be present and optionally administered in the form of salts, hydrates and prodrugs that are converted in vivo into the compounds of the present invention.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

It is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g., potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄) alkyl halides, e.g., methyl, ethyl, iso-propyl and tert-butyl chlorides, bromides and iodides; di (C₁₋₄) alkyl sulfates, e.g., dimethyl, diethyl and diamyl sulfates; (C₁₀₋₁₈) alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water-soluble and oil-soluble compounds of the present invention.

Compounds of the invention further include prodrug derivatives of the compounds. It is noted that in many instances, the prodrugs themselves also fall within the scope of the range of compounds according to the present invention.

Various forms of prodrugs are well known in the art. For examples of such prodrug derivatives, see, e.g.,
a) Design of Prodrugs, Bundgaard, A. Ed., Elsevier, 1985 and Method in Enzymology, Widder, K. et al., Ed.; Academic, 1985, vol. 42, p. 309-396;
b) Bundgaard, H. “Design and Application of Prodrugs” in A Textbook of Drug Design and Development, Krosgaard-Larsen and H. Bundgaard, Ed., 1991, Chapter 5, p. 113-191; and
c) Bundgaard, H., Advanced Drug Delivery Review, 1992, 8, 1-38.
   Each of which is incorporated herein by reference.

Pharmaceutically acceptable prodrugs of the compounds of this invention include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters.

Compounds of the present invention may also be conveniently prepared, or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

### Compositions Comprising Renin Inhibitors

A wide variety of compositions and administration methods may be used in conjunction with the compounds of the present invention. Such compositions may include, in addition to the compounds of the present invention, conventional pharmaceutical excipients, and other conventional, pharmaceutically inactive agents. Additionally, the compositions may include active agents in addition to the compounds of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising compounds of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The renin inhibitors and compositions comprising them may be administered or coadministered in any conventional dosage form. Co-administration in the context of this invention is intended to mean the administration of more than one therapeutic agent, one of which includes a renin inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerin, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When compounds according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding compounds according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more compounds according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of an inhibitor of the present invention to reduce renin activity in vivo, thereby treating the disease state of the subject.

Dosage forms or compositions may optionally comprise one or more compounds according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more renin inhibitors, optionally 0.1-95%, and optionally 1-95%.

Salts, preferably sodium salts, of the inhibitors may be prepared with carriers that protect the compound against rapid elimination from the body, such as time release formulations or coatings. The formulations may further include other active compounds to obtain desired combinations of properties.

### Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain variation of the above embodiments and variations, compounds according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water-soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The compounds of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising compounds of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

### Injectables, Solutions, and Emulsions

The present invention is also directed to compositions designed to administer the compounds of the present invention by parenteral administration, generally characterized by subcutaneous, intramuscular or intravenous injection. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions includes EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of an inhibitor in the parenteral formulation may be adjusted so that an injection administers a pharmaceutically effective amount sufficient to produce the desired pharmacological effect. The exact concentration of an inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is known and practiced in the art.

Injectables may be designed for local and systemic administration. Typically a therapeutically effective dosage is formulated to contain a concentration of at least about 0.1% w/w up to about 90% w/w or more, preferably more than 1% w/w of the renin inhibitor to the treated tissue(s). The inhibitor may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The renin inhibitor may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

### Lyophilized Powders

The compounds of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a renin inhibitor is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the inhibitor.

### Topical Administration

The compounds of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The renin inhibitors may be formulated as aerosols for topical application, such as by inhalation (see, U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the renin inhibitor alone or in combination with other pharmaceutically acceptable excipients can also be administered.

### Formulations for Other Routes of Administrations

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

### Examples of Formulations

The following are particular examples of oral, intravenous and tablet formulations that may optionally be used with compounds of the present invention. It is noted that these formulations may be varied depending on the particular compound being used and the indication for which the formulation is going to be used.

ORAL FORMULATION

| | | |
|---|---|---|
| Compound of the Present Invention | 10-100 | mg |
| Citric Acid Monohydrate | 105 | mg |
| Sodium Hydroxide | 18 | mg |
| Flavoring | | |

| | |
|---|---|
| Water | q.s. to 100 mL |

INTRAVENOUS FORMULATION

| Compound of the Present Invention | 0.1-10 | mg |
|---|---|---|
| Dextrose Monohydrate | q.s. to make isotonic | |
| Citric Acid Monohydrate | l.05 | mg |
| Sodium Hydroxide | 0.18 | mg |

| | |
|---|---|
| Water for Injection | q.s. to 1.0 mL |

TABLET FORMULATION

| | |
|---|---|
| Compound of the Present Invention | 1% |
| Microcrystalline Cellulose | 73% |
| Stearic Acid | 25% |
| Colloidal Silica | 1% |

### Kits Comprising Renin Inhibitors

The invention is also directed to kits and other articles of manufacture for treating diseases associated with Renin. It is noted that diseases are intended to cover all conditions for which the renin possess activity that contributes to the pathology and/or symptomology of the condition.

In one variation of the above embodiments and variations, a kit is provided that comprises a composition comprising at least one inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another variation of the above embodiments and variations, an article of manufacture is provided that comprises a composition comprising at least one inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific variation of the above embodiments and variations of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### Combination Therapy

A wide variety of therapeutic agents may have a therapeutic additive or synergistic effect with renin inhibitors according to the present invention. Such therapeutic agents may additively or synergistically combine with the renin inhibitors to reduce or alleviate the effects and symptoms of cardiovascular disease.

In one variation of the above embodiments and variations, a method is provided for treating cardiovascular disease comprising treating cells with a compound according to the present invention in combination with an aldosterone receptor antagonist, wherein the cells are treated with the compound according to the present invention before, at the same time, and/or after the cells are treated with the aldosterone receptor antagonist, referred to herein as combination therapy. It is noted that treatment of one agent before another is referred to herein as sequential therapy, even if the agents are also administered together. It is noted that combination therapy is intended to cover when agents are administered before or after each other (sequential therapy) as well as when the agents are administered at the same time.

### Preparation of Renin Inhibitors

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (i.e., enantiomers and diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) and can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet and Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this Application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art. For example, appropriate prodrugs can be prepared by reacting a non-derivatized compound with a suitable carbamylating agent (e.g., 1,1-acyloxyalkylcarbonochloridate, para-nitrophenyl carbonate, or the like) or an acylating agent. Further examples of methods of making prodrugs are described in Saulnier et al. Bioorganic and Medicinal Chemistry Letters, 1994, Vol. 4, p. 1985. Those of ordinary skill in the art have the knowledge and means to accomplish this without undue experimentation.

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3rd edition, John Wiley & Sons, Inc. 1999.

Compounds according to the present invention may be conveniently prepared or formed during the process of the invention, as solvates (e.g., hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet and Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| μL (microliters) | Ac (acetyl) |
| atm (atmosphere) | ATP (Adenosine Triphophatase) |
| BOC (tert-butyloxycarbonyl) | BOP (bis(2-oxo-3-oxazolidinyl)phosphinic chloride) |
| Brij35 (polyoxyethyleneglycol dodecyl ether) | BSA (Bovine Serum Albumin) |
| CBZ (benzyloxycarbonyl) | CDI (1,1-carbonyldiimidazole) |
| DCC (dicyclohexylcarbodiimide) | DCE (dichloroethane) |
| DCM (dichloromethane) | DMAP (4-dimethylaminopyridine) |
| DME (1,2-dimethoxyethane) | DMF (N,N-dimethylformamide) |
| DMPU (N,N′-dimethylpropyleneurea) | DMSO (dimethylsulfoxide) |
| DTT (dithiothreitol) | EDCI (ethylcarbodiimide hydrochloride) |
| EDTA (Ethylenediaminetetraacetic acid) | Et (ethyl) |
| Et₂O (diethyl ether) | EtOAc (ethyl acetate) |
| FMOC (9-fluorenylmethoxycarbonyl) | g (grams) |
| h (hours) | HOAc or AcOH (acetic acid) |
| HOBT (1-hydroxybenzotriazole) | HOSu (N-hydroxysuccinimide) |
| HPLC (high pressure liquid chromatography) | Hz (Hertz) |
| i.v. (intravenous) | IBCF (isobutyl chloroformate) |
| i-PrOH (isopropanol) | L (liters) |
| LAH (lithium aluminium hydride) | M (molar) |
| mCPBA (meta-chloroperbenzoic acid) | Me (methyl) |
| MeOH (methanol) | mg (milligrams) |
| MHz (megahertz) | min (minutes) |
| mL (milliliters) | mM (millimolar) |
| mmol (millimoles) | mol (moles) |
| MOPS (Morpholinepropanesulfonic acid) | mp (melting point) |
| NaOAc (sodium acetate) | NEt₃ (triethylamine) |
| OMe (methoxy) | OTf (O-triflate) |
| OMs (O-mesylate) | psi (pounds per square inch) |
| RP (reverse phase) | RT (ambient temperature) |
| SPA (Scintillation Proximity Assay) | TBAF (tetra-n-butylammonium fluoride) |
| TBS (t-butyldimethylsilyl) | tBu (tert-butyl) |
| TEA (triethylamine) | TFA (trifluoroacetic acid) |
| TFAA (trifluoroacetic anhydride) | THF (tetrahydrofuran) |
| TIPS (triisopropylsilyl) | TLC (thin layer chromatography) |
| TMS (trimethylsilyl) | TMSE (2-(trimethylsilyl)ethyl) |
| Tr (retention time) | |

All references to ether or Et₂O are to diethyl ether; and brine refers to a saturated aqueous solution of NaCl. Unless otherwise indicated, all temperatures are expressed in ° C. (degrees Centigrade). All reactions are conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad). When two rotomers are observed, the combined NMR spectra are presented.

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosures of all documents cited throughout this application are incorporated herein by reference.

A. Synthetic Schemes for Compounds of the Present Invention

Compounds according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, John Wiley and Sons, 1991.

General synthetic routes for producing compounds of the present invention are shown in the schemes below. The various substituents may be selected from among the various substituents otherwise taught herein.

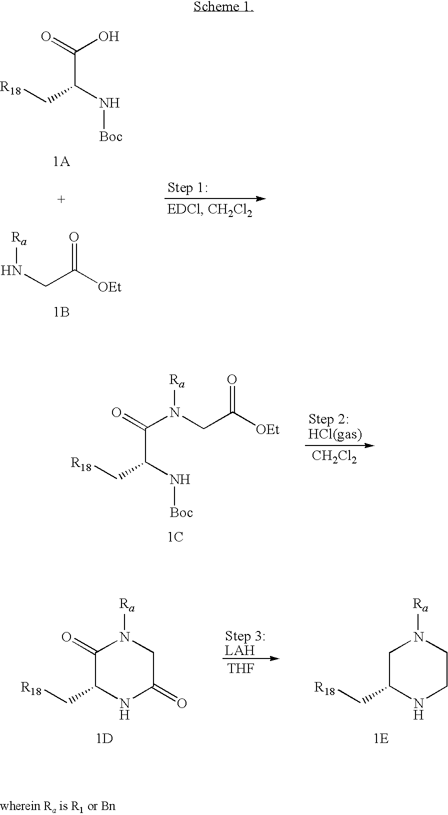

N-Boc protected starting material 1A (1.0 equiv.) can be coupled to N—F,-glycine ethyl ester 1B (1.0 equiv.) using ethylcarbodiimide hydrochloride (EDCI) (1.1-1.5 equiv.) and HOBt (1.1-1.5 equiv.) in dichloromethane (0.1-0.5 M) (Step 1). The Boc group can be removed upon bubbling of HCl gas through the cold (0-15° C.) solution of the resulting amide 1C in dichloromethane (0.2-0.5 M). Subsequent cyclization upon removal of the solvent can afford the dioxopiperazine derivative 1D (Step 2), which can be reduced to the corresponding diamine 1E with lithium aluminum hydride (LAH) (2-3 equiv.) in THF (0.2-0.4 M) at reflux for 1-24 h (Step 3).

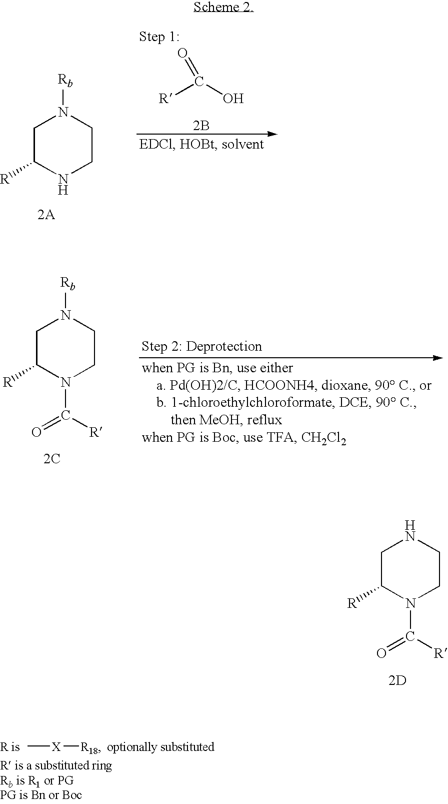

An amine component 2A (1.0 equiv.) can be coupled to cyclic substituted carboxylic acids 2B (1.0-2.0 equiv.) under the standard EDCI (1.0-1.5 equiv.)/HOBt (1.0-1.5 equiv.) conditions in dichloromethane or DMF (0.1-0.5 M) at 0-90° C. (Step 1) forming the corresponding amide 2C. The protective group, if it is benzyl, can be removed (Step 2) using one of the following conditions: (a) Pd(OH)₂/HCOONH₄/dioxane at 90° C. for 0.5-24 h, or (b) 1-chloroethylchloroformate, dichloroethane (DCE), 90° C. for 1-3 h, then MeOH, 20-65° C. for 0.1-2 h. The protective group, if it is Boc, can be removed with excess TFA in dichloromethane (room temperature, 0.1-18 h). The product 2D can be purified by HPLC (acetonitrile-water, TFA buffered) or flash column chromatography on silica gel.

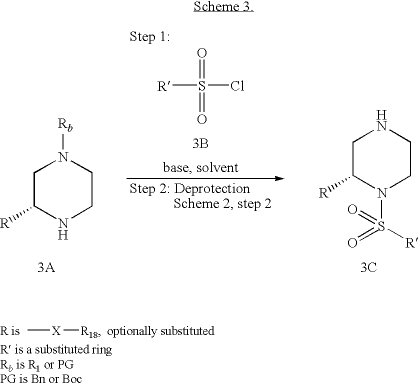

An amine component (1.0 equiv.) 3A can be reacted with a cyclic substituted sulfonyl chloride 3B (1.0-3.0 equiv.) using base (e.g. NEt₃ or pyridine) in an appropriate solvent (e.g. dichloromethane, 0.1-0.5 M). The resulting sulfonamide can be deprotected in a manner identical to that in Scheme 2, Step 2 yielding compound 3C.

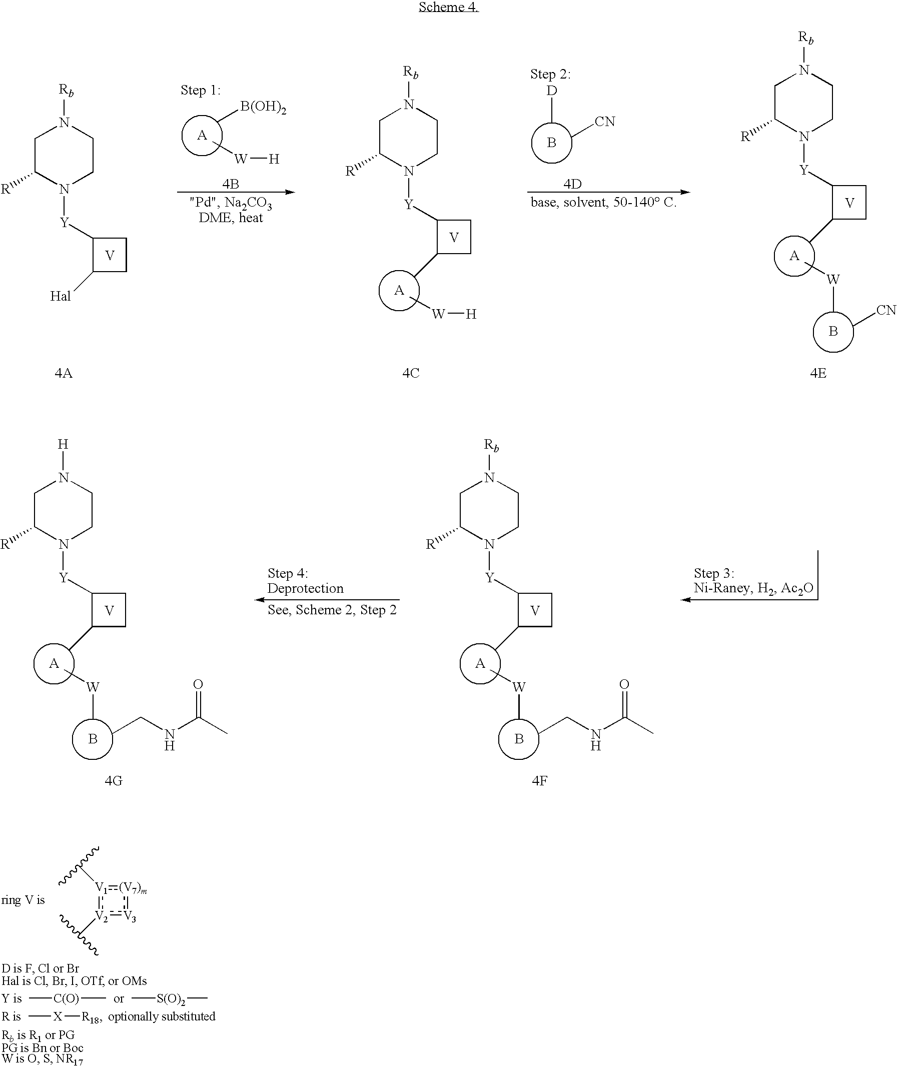

Halogenated cyclic compound 4A (where Y is —C(O)— or —S(O)₂—, and R is a substituted ring) produced by the methods of Scheme 2 and 3 can be reacted with a cyclic substituted boronic acid 4B in a palladium-catalyzed (e.g. Pd(PPh₃)₄, 0.05-0.5 equiv.) coupling, using base (e.g. aq. Na₂CO₃, 2-4 equiv.) in DME (0.05-0.5 M) at 20-120° C. (Step 1). The resulting compound 4C (1.0 equiv.) can then be used in the nucleophilic displacement (Step 2) of the cyclic substituted halide 4D (1-10 equiv.) using base (e.g. K₂CO₃ or Cs₂CO₃) in acetone or DMF (0.1-1.0 M) upon heating to 50-140° C. for 0.5h to 3d. The resulting nitrile 4E (1 equiv.) can be reduced to the primary amine and in situ converted to an acetyl-derivative using H₂ (30-100 psi) and Ni-Raney (excess) in Ac₂O (0.05-0.2 M) to afford compound 4F (Step 3). Finally, deprotection of the secondary amine (Step 4, as described in Scheme 2, Step 2) can afford compound 4G.

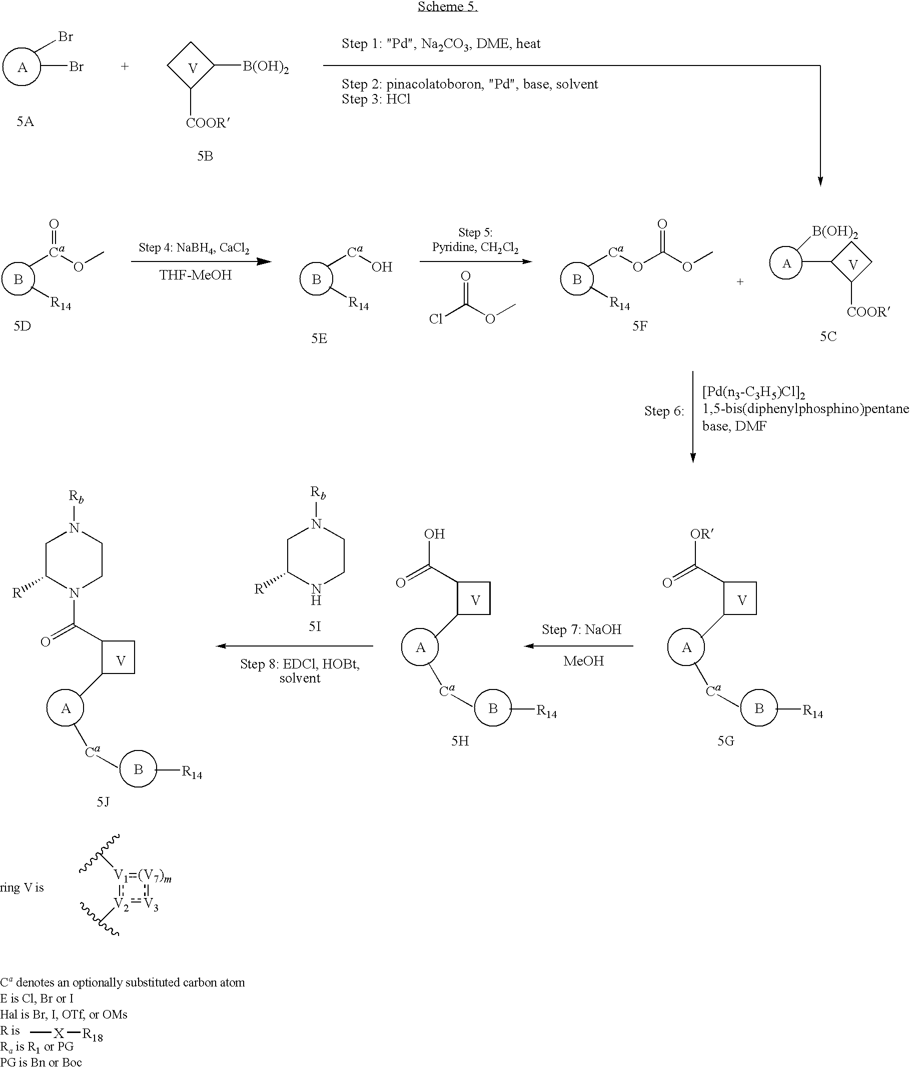

Scheme 5 provides a route for preparing compounds 5J. C^{a} of compound 5J denotes a carbon which may be substituted or unsubstituted.

Compound 5J where C^{a} is an unsubstituted methylene (—CH₂—) may be prepared according to Scheme 5. In Step 1, the dibromo derivative 5A may be coupled to the boronic acid 5B in a palladium catalyzed reaction as described in Scheme 4, Step 1. The reaction product can then be derivatized with pinacolatoboron, in a similar palladium catalyzed reaction to form the borate ester which may be hydrolyzed by HCl forming the corresponding boronic acid derivative 5C.

Separately, in Step 4, the ester starting material 5D (1 equiv.) can be reduced with NaBH₄ in the presence of CaCl₂ (2 equiv.) in THF-MeOH (0.05-0.15 M). The resulting primary alcohol 5E (1 equiv.) can be converted to the carbonate 5F with methylchloroformate (1.0 equiv.) and pyridine (2-5 equiv.) in dichloromethane (0.1-0.5 M) at 0-30° C. (Step 5). The carbonate 5F (1.0 equiv.) can then be used in the Suzuki coupling (Step 6) with the boronic acid derivative 5C (0.7-3 equiv.) using allylpalladium (II) chloride dimer (0.02-0.2 equiv.), 1,5-bis-(diphenylphosphino)pentane (0.04-0.4 equiv.), and a base (e.g. K₂CO₃, 2.2 equiv.) in DMF (1 M). The resulting ester 5G can be saponified with a base (e.g. excess 1M aq. NaOH), in MeOH (0.1-0.3 M) (Step 7). The resulting acid 5H (1 equiv.) can be reacted with the amine component 5I (1-1.5 equiv.) using standard coupling conditions (Step 8, as described in Scheme 2, Step 1). If desirable, the protection group, Boc or Bn, can be removed as described in Scheme 2, Step 2.

For preparing compounds 5J where C^{a} is a mono-substituted methylene (e.g., —CHR₁₅ and R₁₅ is not hydrogen), the primary alcohol 5E may be oxidized to a secondary alcohol (R₁₄—Ring B-CHR₁₅—OH) prior to being converted to a carbonate in Step 5. The primary alcohol 5E can be oxidized by conventional methods such as a Swern oxidation to produce an aldehyde, and a Grignard reagent R₁₅MgE (E=Cl, Br or I) can then be added to the aldehyde to provide the secondary alcohol 5E′.

For preparing compounds 5J where C^{a} is a di-substituted methylene (e.g., —CR₁₅R₁₆— and R₁₅ and R₁₆ both represent groups other than hydrogen), the secondary alcohol can be further oxidized by conventional means such as a Swern oxidation to provide a ketone, and a second Grignard reagent, R₁₆MgE, can be added to the ketone to provide a tertiary alcohol (R₁₄—Ring B-CR₁₅R₁₆—OH), where R₁₅ and R₁₆ are both not hydrogen. The tertiary alcohol can then be converted to a carbonate 5J (Step 5); and progressing through the subsequent synthesis steps lead to the formation of compound 5J, where rings A and B are separated by a substituted methylene CR₁₅R₁₆ as illustrated in Scheme 5.

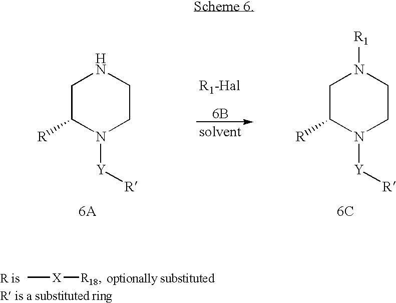

Deprotected inhibitors of the above schemes may be converted to prodrug 6C by coupling a masking group (R₁) to the amine component. The amine component 6A (1.0 equiv.) can be reacted with an activated derivative of the masking group (1-3 equiv.), such as a Hal-derivative 6B (Hal is Cl, Br, OTf, OMs, or the like), in an appropriate solvent (e.g. dichloromethane, DMF, acetone, 0.1-0.5 M), optionally using base (e.g. NEt₃ or pyridine). For example, R₁ can be alkyl, e.g. methyl or ethyl, and can be attached by reaction with the corresponding alkyl chloride, bromide or iodide. Further example, R₁ can be an acyl group and can be attached by reaction with the corresponding acyl chloride, bromide or iodide. Methods and conditions for attaching various R₁ groups are well known in the art, and may be found, for example, in T W Greene, Protective Groups in Organic Synthesis, 4th ed.

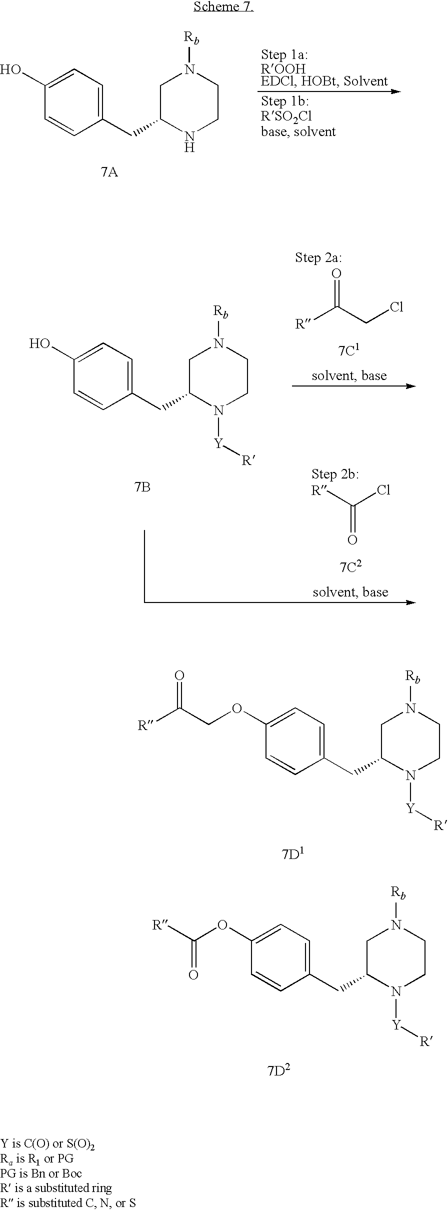

The cyclic substituents on the amine component may be functionalized. The amine component 7A can be acylated as described in Scheme 2 or Scheme 3 (Step 1a or 1b, respectively) depending on whether Y is C(O) or S(O)₂. The resulting phenol 7B (1 equiv.) can then be functionalized with various electrophiles (1-10 equiv.) (Step 2). For example, phenol 7B may react with α-halocarbonyl-containing compound 7C¹ (3-10 equiv.) using base (e.g. K₂CO₃, 3-5 equiv.) in acetone or DMF (0.1-1.0 M) at 50-130° C. (Step 2a) to afford compound 7D¹. Also, 7B may react with acid chloride/carbamoyl chloride 7C² (1-10 equiv.) in pyridine (0.2-1.2 M) at 0-50° C. (Step 2b) to afford compound 7D² respectively. Compounds 7D¹ and 7D² may be deprotected (when R_{b} is Bn or Boc) as described in Scheme 2, Step 2.

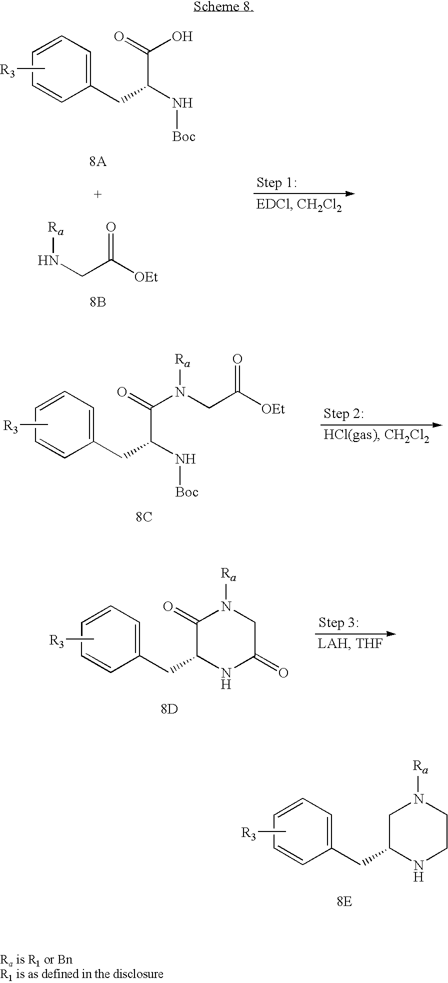

The amine component 8E may be prepared by the synthetic route as described in Scheme 1. Specifically, in connection to Scheme 8, the coupling of N-Boc protected phenylalanine derivative 8A to N—R₃-glycine ethyl ester 8B can afford amide 8C. The removal of the Boc group and subsequent cyclization of 8C to the dioxopiperazine derivative 8D and its LAH reduction then yields the diamine product 8L. The diamine product 8L may be deprotected (when Rₐ is Bn) by the procedure described in Scheme 2, Step 2.

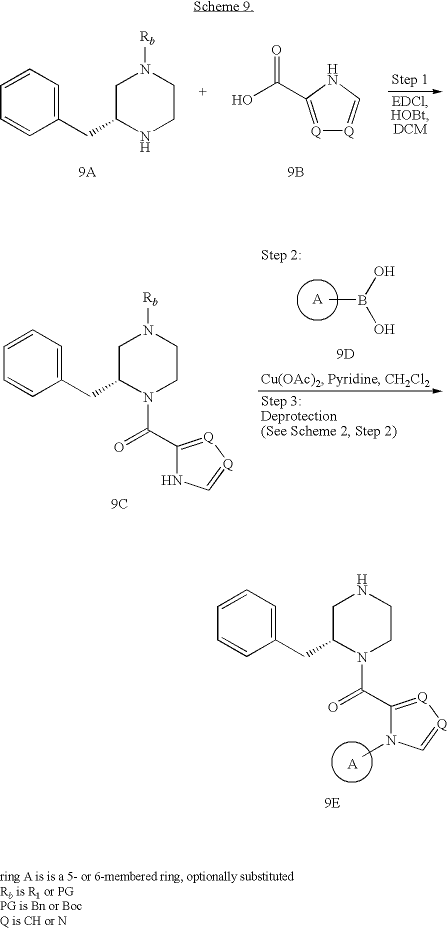

In connection to Scheme 9, the amine component 9A can be coupled to N—H containing heterocyclic carboxylic acid 9B (Step 1) as described in Scheme 2, Step 1. The heterocycle 9C (1.0 equiv.) can be coupled to various boronic acids, e.g. 9D (2-4 equiv.) using Cu(OAc)₂ (3-10 equiv.) in the presence of base (TEA or pyridine, 3-10 equiv.) in dichloromethane (0.1-0.3 M) at room temperature for 1-7 d. Deprotection as described in Scheme 2, Step 2 can afford the desired amine 9E (Steps 2 and 3).

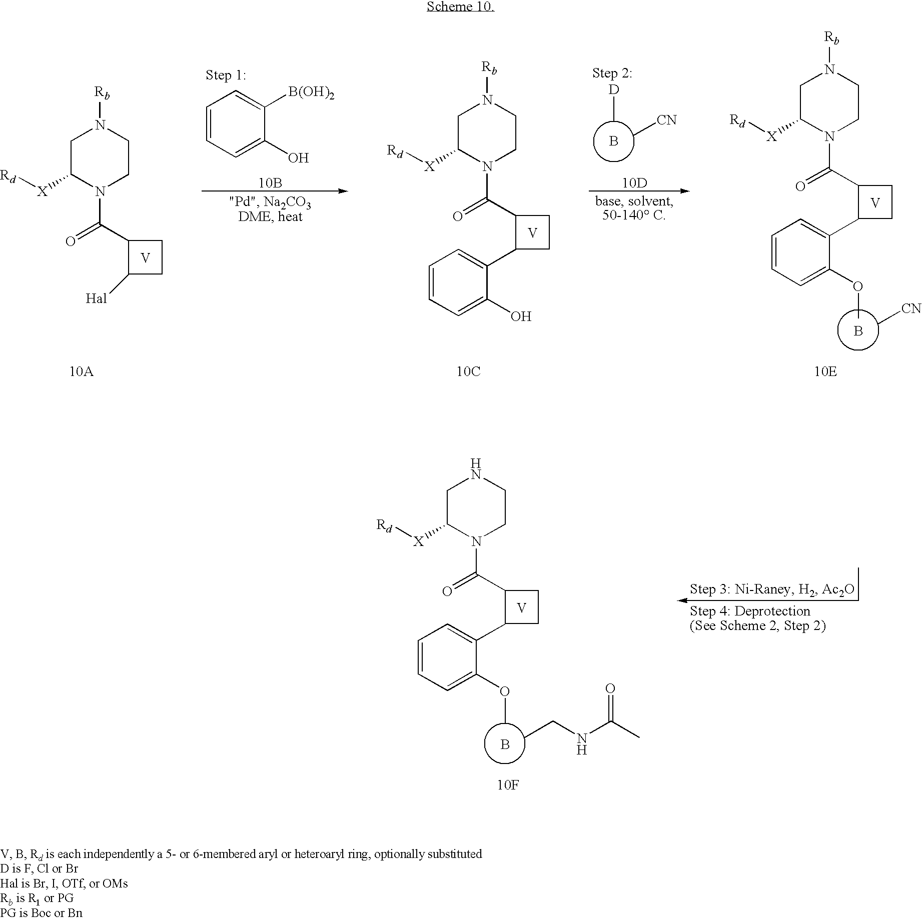

Many target compounds 10F may be prepared via the route described in Scheme 4. Specifically, in connection to Scheme 10, the coupling of haloaryl compound 10A with 2-hydroxyphenylboronic acid (10B) forms phenol 10C which is then used in the nucleophilic displacement (Step 2) of the aryl or heteroaryl halide 10D forming nitrile 10E. 10E can be reduced to the primary amine and in situ converted to an acetyl-derivative (Step 3). The secondary amine functionality may then be deprotected (Step 4) as described in Scheme 2, Step 2 to afford the target compounds 10F.

In each of the above reaction procedures or schemes, the various substituents may be selected from among the various substituents otherwise taught herein.

Chiral components can be separated and purified using any of a variety of techniques known to those skilled in the art. For example, chiral components can be purified using supercritical fluid chromatography (SFC). In one particular variation, chiral analytical SFC/MS analyses are conducted using a Berger analytical SFC system (AutoChem, Newark, Del.) which consists of a Berger SFC dual pump fluid control module with a Berger FCM 1100/1200 supercritical fluid pump and FCM 1200 modifier fluid pump, a Berger TCM 2000 oven, and an Alcott 718 autosampler. The integrated system can be controlled by BI-SFC Chemstation software version 3.4. Detection can be accomplished with a Waters ZQ 2000 detector operated in positive mode with an ESI interface and a scan range from 200-800 Da with 0.5 second per scan. Chromatographic separations can be performed on a ChiralPak AD-H, ChiralPak AS-H, ChiralCel OD-H, or ChiralCel OJ-H column (5μ, 4.6×250 mm; Chiral Technologies, Inc. West Chester, Pa.) with 10 to 40% methanol as the modifier and with or without ammonium acetate (10 mM). Any of a variety of flow rates can be utilized including, for example, 1.5 or 3.5 mL/min with an inlet pressure set at 100 bar. Additionally, a variety of sample injection conditions can be used including, for example, sample injections of either 5 or 10 μL in methanol at 0.1 mg/mL in concentration.

In another variation, preparative chiral separations are performed using a Berger MultiGram II SFC purification system. For example, samples can be loaded onto a ChiralPak AD column (21×250 mm, 10g). In particular variations, the flow rate for separation can be 70 mL/min, the injection volume up to 2 mL, and the inlet pressure set at 130 bar. Stacked injections can be applied to increase the efficiency.

Description of the syntheses of particular compounds according to the present invention based on the above reaction schemes as set forth herein.

The present invention is further exemplified, but not limited, by examples provided below that describe the synthesis of particular compounds according to the invention.

### Biological Testing

The activity of compounds as renin inhibitors may be assayed in vitro, in vivo or in a cell line. Example D below provides an in vitro enzymatic activity assay for activity against Renin.

Test compounds in varying concentrations may be reacted with recombinant human renin in the presence of substrate, e.g., QXL520-γ-Abu-Ile-His-Pro-Phe-His-Leu-Val-Ile-His-Thr-Lys (HiLyteFluo488)-Arg-OH (Anaspec, San Jose, Calif.). The reaction can be followed kinetically using fluorescence (excitation λ=485 nm; emission λ=538 nm). Inhibition constants (IC₅₀) may be calculated by non-linear curve fitting of the compound concentrations and fluorescence intensities to the standard IC₅₀ equation. IC₅₀ values for selected compounds of the present invention are given in Table 1.

### EXAMPLE

### Example 1

### ((R)-2-benzylpiperazin-1-yl)(3-(2-phenoxyphenyl)thiophen-2-yl)methanone

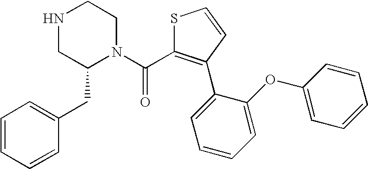

### 1A. (R)-(3-bromothiophen-2-yl)(2,4-dibenzylpiperazin-1-yl)methanone

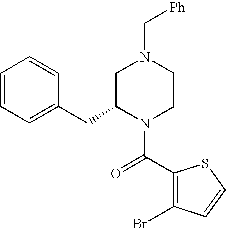

(R)-1,3-dibenzylpiperazine (2.66 g, 10.0 mmol) and 3-bromothiophene-2-carboxylic acid (2.30 g, 11.1 mmol) were dissolved in dichloromethane (50 mL). EDCI (2.30 g, 12.0 mmol) and HOBt (1.80 g, 11.8 mmol) were added and the reaction mixture was stirred at room temperature overnight. The reaction mixture concentrated in vacuo, diluted with ethyl acetate (100 mL) and washed with water (50 mL), NaHCO₃ (sat. aq., 50 mL), dried (MgSO₄) and concentrated in vacuo. The crude material was purified by flash column chromatography on silica gel (90g SiO₂, hexanes-ethyl acetate-dichloromethane 6:1:1, 1200 mL) to afford the title compound as a yellow viscous oil (3.19 g, 70%). ESI-MS: m/z 455.3 (M+H)⁺.

### 1B. ((R)-2,4-dibenzylpiperazin-1-yl)(3-(2-phenoxyphenyl)thiophen-2-yl)methanone

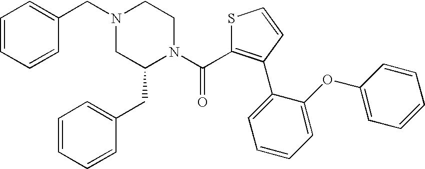

(R)-(3-bromothiophen-2-yl)(2,4-dibenzylpiperazin-1-yl)methanone (227.2 mg, 0.500 mmol) and 2-phenoxyphenylboronic acid (128.4 mg, 0.600 mmol) were suspended in Na₂CO₃ (2M aq., 0.9 mL, 1.80 mmol), EtOH (2 mL) and benzene (2 mL) under nitrogen atmosphere. Pd(PPh₃)₄ (58.0 mg, 0.0502 mmol) was added and the reaction mixture was tightly sealed and stirred at 90° C. overnight. It was extracted with ethyl acetate (2×3 mL) and the combined organic extracts were washed with brine (5 mL), dried (MgSO₄), filtered and concentrated in vacuo to afford the title compound as a red oil (0.260 g, 95%). ESI-MS: m/z 545.4 (M+H)⁺.

### 1C. ((R)-2-benzylpiperazin-1-yl)(3-(2-phenoxyphenyl)thiophen-2-yl)methanone

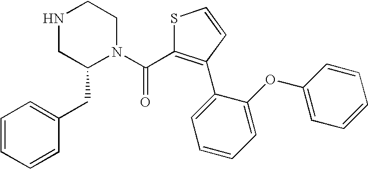

((R)-2,4-dibenzylpiperazin-1-yl)(3-(2-phenoxyphenyl)thiophen-2-yl)methanone (0.260 g, 0.477 mmol) was dissolved in dichloroethane (2 mL) and cooled to 0° C. 1-Chloroethyl chloroformate (1.00 mL, 5.27 mmol) was added and the reaction mixture was heated under reflux (90° C.) in a closed vial for 3 h. The mixture was concentrated in vacuo at 35° C. to remove the excess chloroformate and dissolved in MeOH (3 mL). It was then heated to reflux for 30 min, cooled, concentrated in vacuo and the crude product was purified by HPLC (25-40% acetonitrile in water, buffered by 0.05% TFA) to afford the title compound as a white solid (57.0 mg, 26%). ESI-MS: m/z 455.3 (M+H)⁺.

### Example 2

### ((S)-2-benzylpiperazin-1-yl)(2-phenylcyclopropyl)methanone

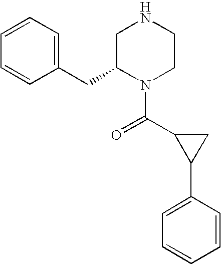

The title compound was prepared as described for Example 1-A,C. ESI-MS: m/z 321.4 (M+H)⁺.

### Example 3

### (R)-(2-benzylpiperazin-1-yl)(3′,4′-dimethylbiphenyl-2-yl)methanone

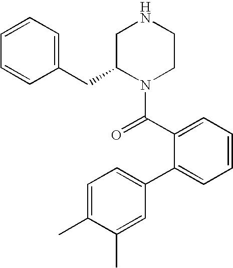

### 3A. (R)-(2,4-dibenzylpiperazin-1-yl)(3′,4′-dimethylbiphenyl-2-yl)methanone

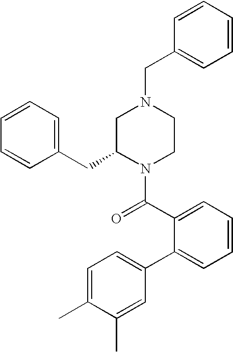

The title compound was prepared as described for Example 1-A using 3′,4′-dimethylbiphenyl-2-carboxylic acid instead of 3-bromothiophene-2-carboxylic acid. ESI-MS: m/z 475.4 (M+H)⁺.

### 3B. (R)-(2-benzylpiperazin-1-yl)(3′,4′-dimethylbiphenyl-2-yl)methanone

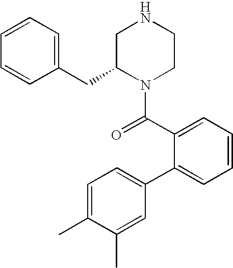

(R)-(2,4-dibenzylpiperazin-1-yl)(3′,4′-dimethylbiphenyl-2-yl)methanone (453 mg, 0.954 mmol) was dissolved in dioxane (7 mL), and Pd(OH)₂/C (20% w/w, 1.50 g) was added to the reaction mixture followed by ammonium formate (1.50 g). The reaction mixture was capped and stirred at 90° C. for 16 h. It was cooled to room temperature and filtered through a short plug of celite. The plug was washed with ethyl acetate (20 mL) and the filtrate was concentrated in vacuo. The crude product was purified by HPLC (25-40% acetonitrile in water, TFA buffered) to afford the title compound as a white solid (TFA salt; 104 mg, 22%). ESI-MS: m/z 385.3 (M+H)⁺.

### Example 4

### (R)-(2-benzylpiperazin-1-yl)(biphenyl-2-yl)methanone

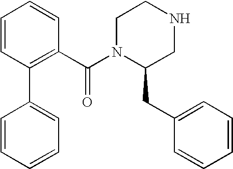

The title compound was prepared as described for Example 3. ESI-MS: m/z 357.4 (M+H)⁺.

### Example 5

### ((R)-2-benzylpiperazin-1-yl)(2′-chlorobiphenyl-2-yl)methanone

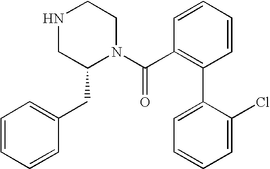

The title compound was prepared as described for Example 1A and C. ESI-MS: m/z 391.4 (M+H)⁺.

### Example 6

### (R)-(2-benzylpiperazin-1-yl)(1-phenyl-1H-pyrazol-5-yl)methanone

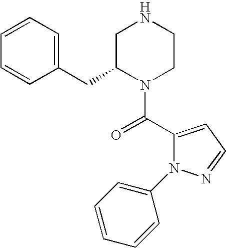

The title compound was prepared as described for Example 3. ESI-MS: m/z 347.4 (M+H)⁺.

### Example 7

### (R)-(2-benzylpiperazin-1-yl)(5-phenyloxazol-4-yl)methanone

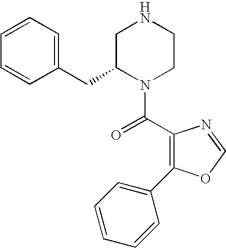

The title compound was prepared as described for Example 3. ESI-MS: m/z 348.4 (M+H)⁺.

### Example 8

### ((R)-2-benzylpiperazin-1-yl)(1-(2-(trifluoromethyl)phenyl)-1H-imidazol-2-yl)methanone

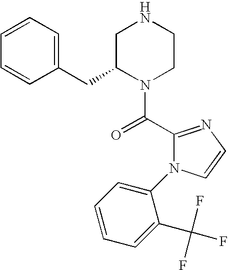

The title compound was prepared as described for Example 3. ESI-MS: m/z 415.4 (M+H)⁺.

### Example 9

### 2-(2-(2-((R)-2-benzylpiperazine-1-carbonyl)thiophen-3-yl)phenoxy)-4,6-dimethylnicotinonitrile

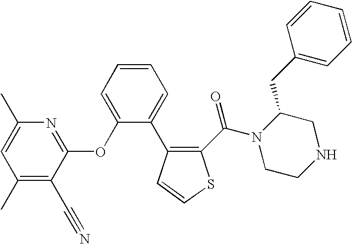

### 9A. (R)-tert-butyl 3-benzyl-4-(3-bromothiophene-2-carbonyl)piperazine-1-carboxylate

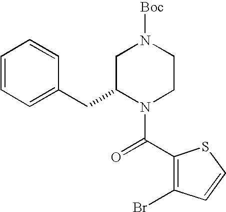

(R)-tert-butyl 3-benzylpiperazine-1-carboxylate (Ref: WO02089738) (2.77 g, 10.0 mmol) and 3-bromothiophene-2-carboxylic acid (2.30 g, 11.1 mmol) were dissolved in dichloromethane (20 mL). EDCI (2.30 g, 12.0 mmol) and HOBt (1.80 g, 11.8 mmol) were added and the reaction mixture was stirred at room temperature overnight. The reaction mixture concentrated in vacuo, diluted with ethyl acetate (100 mL), washed with water (50 mL), NaHCO₃ (sat. aq., 50 mL), dried (MgSO₄) and concentrated n vacuo. The crude material was purified by flash column chromatography on silica gel (120g SiO₂, hexanes-ethyl acetate 3:1 (1 L), then 2:1 (500 mL)) to afford the title compound as a yellow viscous oil (3.45 g, 74%). ESI-MS: m/z 465.2 (M+H)⁺.

### 9B. (3R)-tert-butyl 3-benzyl-4-(3-(2-hydroxyphenyl)thiophene-2-carbonyl)piperazine-1-carboxylate

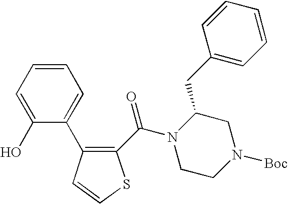

(R)-tert-butyl 3-benzyl-4-(3-bromothiophene-2-carbonyl)piperazine-1-carboxylate (2.00 g, 4.30 mmol) and 2-hydroxybenzeneboronic acid (0.720 g, 5.22 mmol) were suspended in DME (5 mL) and Na₂CO₃ (2M aq., 5 mL). The reaction mixture was degassed and Pd(PPh₃)₄ (400 mg, 0.346 mmol) was added. The reaction vial was capped and heated at 90° C. for 20 h. The reaction mixture was diluted water (5 mL) and extracted with ethyl acetate (2×10 mL). The combined organic layers were washed with water (10 mL), HCl (1N, 10 mL), NaHCO₃ (sat., 10 mL), brine (10 mL), dried (MgSO₄) and concentrated in vacuo. The crude oil was purified by flash column chromatography on silica gel to afford the title compound as an off-white solid (0.821 g, 40%). ESI-MS: m/z 479.4 (M+H)⁺.

### 9C. (3R)-tert-butyl 3-benzyl-4-(3-(2-(3-cyano-4,6-dimethylpyridin-2-yloxy)phenyl)thiophene-2-carbonyl)piperazine-1-carboxylate

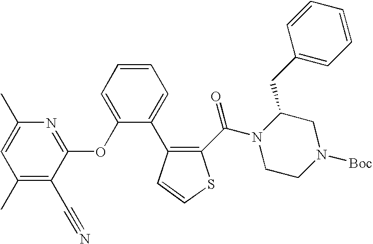

(3R)-tert-butyl 3-benzyl-4-(3-(2-hydroxyphenyl)thiophene-2-carbonyl)piperazine-1-carboxylate (50.0 mg, 0.104 mmol), and 2-chloro-4,6-dimethylnicotinonitrile (100 mg, 0.600 mmol) were dissolved in DMF (0.3 mL). K₂CO₃ was added and the reaction mixture was heated in a closed vial at 90° C. for 1 h. The mixture was cooled to room temperature, diluted with water (3 mL) and extracted with ethyl acetate (4×2 mL). The combined organic extracts were dried (MgSO₄), filtered and concentrated in vacuo at 60° C. to afford the title compound as a dark oil (contains excess 2-chloro-4,6-dimethylnicotinonitrile), which was used in the next step without further purification. ESI-MS: m/z 609.4 (M+H)⁺.

### 9D. 2-(2-(2-((R)-2-benzylpiperazine-1-carbonyl)thiophen-3-yl)phenoxy)-4,6-dimethylnicotinonitrile

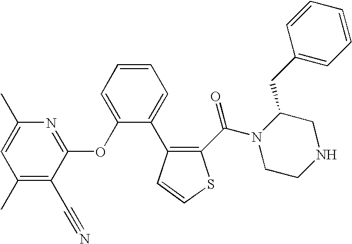

2-(2-(2-((R)-2-benzylpiperazine-1-carbonyl)thiophen-3-yl)phenoxy)-4,6-dimethylnicotinonitrile (crude material from the step C) was dissolved in dichloromethane (2 mL) and TFA (1 mL) and stirred loosely capped for 15 min. The reaction mixture was concentrated in vacuo and the crude product was purified by HPLC to afford 25.0 mg (40%) as a white solid (TFA salt). ESI-MS: m/z 509.4 (M+H)⁺.

### Example 10

### 2-(2-(2-((R)-2-benzylpiperazine-1-carbonyl)thiophen-3-yl)phenoxy)benzonitrile

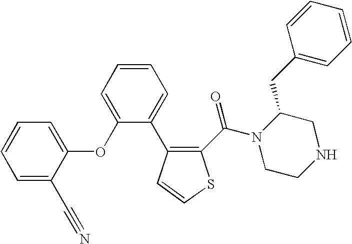

The title compound was prepared as described for Example 9. ESI-MS: m/z 480.3 (M+H)⁺.

### Example 11

### 2-(2-(2-((R)-2-benzylpiperazine-1-carbonyl)thiophen-3-yl)phenoxy)nicotinonitrile

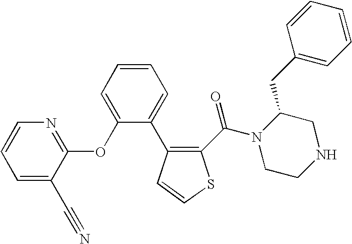

The title compound was prepared as described for Example 9. ESI-MS: m/z 481.4 (M+H)⁺.

### Example 12

### ((R)-2-benzylpiperazin-1-yl)(2-(2-phenoxyphenyl)thiophen-3-yl)methanone

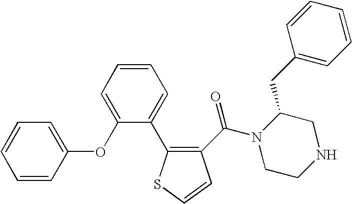

### 12A. (R)-tert-butyl 3-benzyl-4-(2-bromothiophene-3-carbonyl)piperazine-1-carboxylate

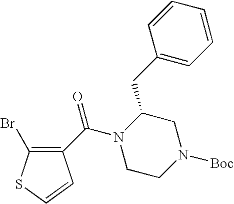

The title compound was prepared as described for Example 9-A. ESI-MS: m/z 465.2 (M+H)⁺.

### 12B. (3R)-tert-butyl 3-benzyl-4-(2-(2-phenoxyphenyl)thiophene-3-carbonyl)piperazine-1-carboxylate

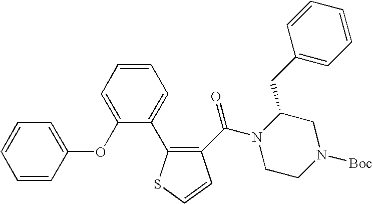

The title compound was prepared as described for Example 1-B. ESI-MS: m/z 555.4 (M+H)⁺.

### 12C. ((R)-2-benzylpiperazin-1-yl)(2-(2-phenoxyphenyl)thiophen-3-yl)methanone

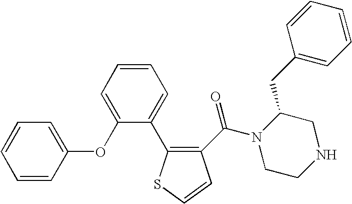

The title compound was prepared as described for Example 9-D. ESI-MS: m/z 455.3 (M+H)⁺.

### Example 13

### ((R)-2-benzylpiperazin-1-yl)(1,3-dimethyl-4-(2-phenoxyphenyl)-1H-pyrazol-5-yl)methanone

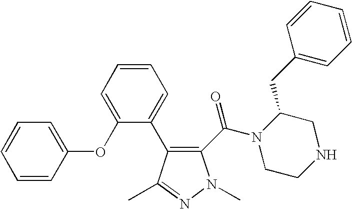

The title compound was prepared as described for Example 12. ESI-MS: m/z 467.4 (M+H)⁺.

### Example 14

### ((R)-2-benzylpiperazin-1-yl)(1-ethyl-3-methyl-4-(2-phenoxyphenyl)-1H-pyrazol-5-yl)methanone

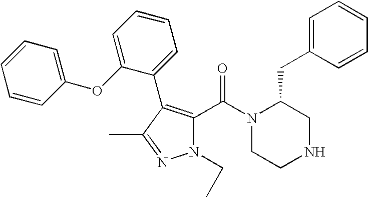

The title compound was prepared as described for Example 12. ESI-MS: m/z 481.4 (M+H)⁺.

### Example 15

### ((R)-2-benzylpiperazin-1-yl)(5-fluoro-2′-phenoxybiphenyl-2-yl)methanone

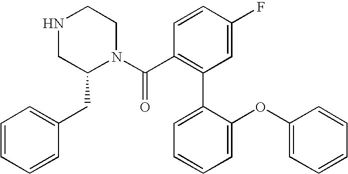

The title compound was prepared as described for Example 12. ESI-MS: m/z 467.4 (M+H)⁺.

### Example 16

### ((R)-2-benzylpiperazin-1-yl)(4-fluoro-2′-phenoxybiphenyl-2-yl)methanone

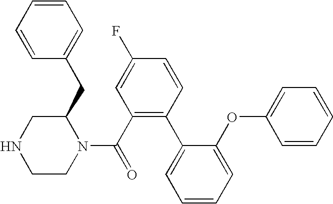

The title compound was prepared as described for Example 12. ESI-MS: m/z 467.4 (M+H)⁺.

### Example 17

### ((R)-2-benzylpiperazin-1-yl)(1-methyl-4-(2-phenoxyphenyl)-1H-pyrazol-3-yl)methanone

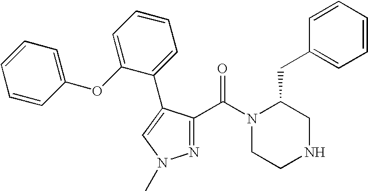

The title compound was prepared as described for Example 12. ESI-MS: m/z 453.4 (M+H)⁺.

### Example 18

### (R)-(3-(1-benzyl-1H-pyrazol-4-yl)thiophen-2-yl)(2-benzylpiperazin-1-yl)methanone

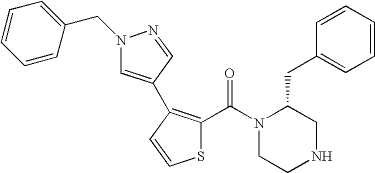

The title compound was prepared as described for Example 12 using 1-benzyl-1H-pyrazol-4-ylboronic acid instead of 2-phenoxyphenylboronic acid. ESI-MS: m/z 443.4 (M+H)⁺.

### Example 19

### ((R)-2-benzylpiperazin-1-yl)(2-(biphenyl-2-yl)-thiophen-3-yl)-methanone

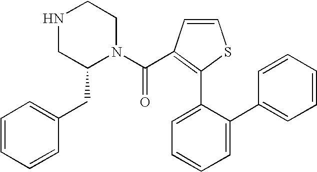

The title compound was prepared as described for Example 12. ESI-MS: m/z 439.3 (M+H)⁺.

### Example 20

### (R)-2-(2-(2-benzylpiperazine-1-carbonyl)phenylthio)benzonitrile

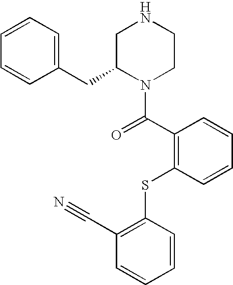

### 20A. (R)-tert-butyl 3-benzyl-4-(2-(2-cyanophenylthio)benzoyl)piperazine-1-carboxylate

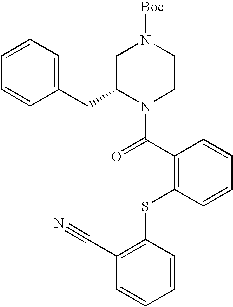

The title compound was prepared by the coupling of (R)-tert-butyl-3-benzylpiperazine-1-carboxyate to 2-(2-cyanophenylthio)benzoic acid as described in Scheme 2. 2-(2-cyanophenylthio)benzoic acid is available commercially. ESI-MS: m/z 514.4 (M+H)⁺.

### 20B. (R)-2-(2-(2-benzylpiperazine-1-carbonyl)phenylthio)benzonitrile

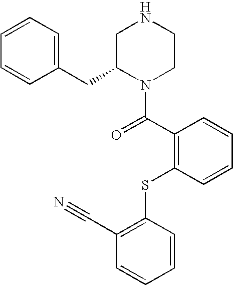

The title compound was prepared as described for Example 9-D. ESI-MS: m/z 414.4 (M+H)⁺.

### Example 21

### (R)-(2-benzylphenyl)(2-benzylpiperazin-1-yl)methanone

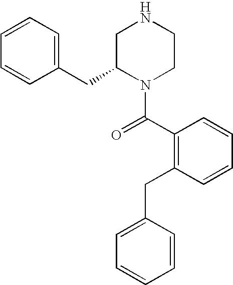

The title compound was prepared as described for Example 20. 2-benzylbenzoic acid is available commercially. ESI-MS: m/z 371.4 (M+H)⁺.

### Example 22

### (R)-(2-benzoylphenyl)(2-benzylpiperazin-1-yl)methanone

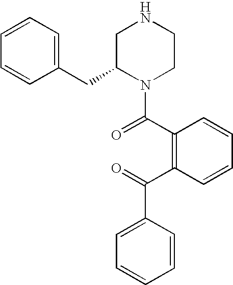

The title compound was prepared as described for Example 20. ESI-MS: m/z 385.4 (M+H)⁺.

### Example 23

### (R)-(2-(benzyloxy)phenyl)(2-benzylpiperazin-1-yl)methanone

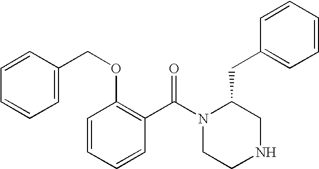

The title compound was prepared as described for Example 20. ESI-MS: m/z 387 (M+H)⁺.

### Example 24

### 1-benzhydryl-4-((R)-2-benzylpiperazine-1-carbonyl)pyrrolidin-2-one

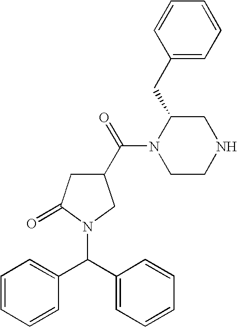

The title compound was prepared as described for Example 20. ESI-MS: m/z 454 (M+H)⁺.

### Example 25

### (R)-(2-benzylpiperazin-1-yl)(2-(4-methoxybenzoyl)phenyl)methanone

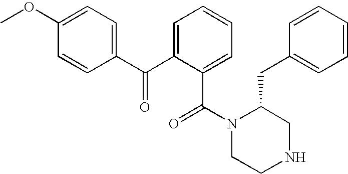

The title compound was prepared as described for Example 20. ESI-MS: m/z 415 (M+H)⁺.

### Example 26

### ((R)-2-benzylpiperazin-1-yl)(1-(naphthalen-1-yl)-1H-imidazol-5-yl)methanone

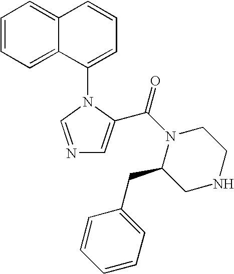

The title compound was prepared as described for Example 20. ESI-MS: m/z 397 (M+H)⁺.

### Example 27

### ((R)-2-benzylpiperazin-1-yl)(1-(biphenyl-2-yl)-1H-imidazol-5-yl)methanone

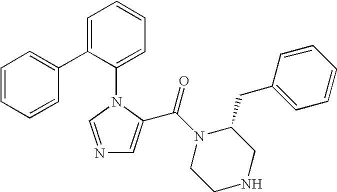

The title compound was prepared as described for Example 20. ESI-MS: m/z 423 (M+H)⁺.

### Example 28

### ((R)-2-benzylpiperazin-1-yl)(2-(3,5-dimethyl-1H-pyrazol-4-yl)phenyl)methanone

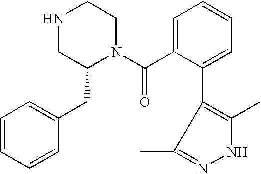

The title compound was prepared as described for Example 20. ESI-MS: m/z 375 (M+H)⁺.

### Example 29

### (R)-(2-benzylpiperazin-1-yl)(3-methyl-5-phenylisoxazol-4-yl)methanone

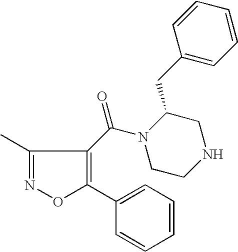

The title compound was prepared as described for Example 20. ESI-MS: m/z 362 (M+H)⁺.

### Example 30

### (R)-(2-benzylpiperazin-1-yl)(5-phenyl-2-(pyridin-3-yl)thiazol-4-yl)methanone

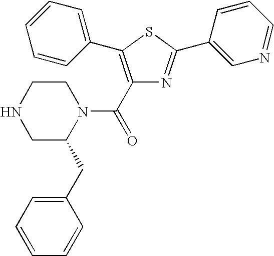

The title compound was prepared as described for Example 20. ESI-MS: m/z 441 (M+H)⁺.

### Example 31

### (R)-(2-benzylpiperazin-1-yl)(4′-fluorobiphenyl-2-yl)methanone

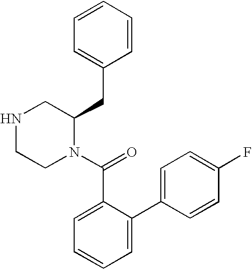

The title compound was prepared as described for Example 20. ESI-MS: m/z 375 (M+H)⁺.

### Example 32

### (R)-(2-(benzo[d]thiazol-2-yl)phenyl)(2-benzylpiperazin-1-yl)methanone

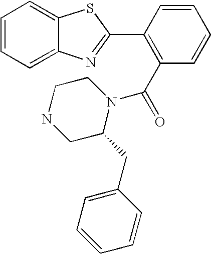

The title compound was prepared as described for Example 20. ESI-MS: m/z 414 (M+H)⁺.

### Example 33

### ((R)-2-benzylpiperazin-1-yl)(3-(2-chlorophenyl)-5-cyclopropylisoxazol-4-yl)methanone

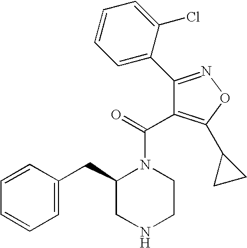

The title compound was prepared as described for Example 20. ESI-MS: m/z 422 (M+H)⁺.

### Example 34

### (R)-(6-benzyl-2-methyl-6H-thieno[2,3-b]pyrrol-5-yl)(2-benzylpiperazin-1-yl)methanone

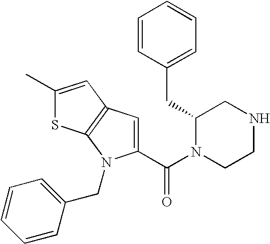

The title compound was prepared as described for Example 20. ESI-MS: m/z 430 (M+H)⁺.

### Example 35

### (R)-(2-benzylpiperazin-1-yl)(2,2-dimethyl-2,3-dihydrobenzofuran-7-yl)methanone

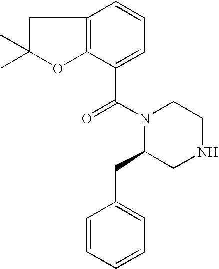

The title compound was prepared as described for Example 20. ESI-MS: m/z 351 (M+H)⁺.

### Example 36

### (R)-(2-benzylpiperazin-1-yl)(4-(4-chlorophenyl)thiophen-2-yl)methanone

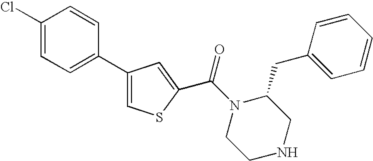

The title compound was prepared as described for Example 20. ESI-MS: m/z 397 (M+H)⁺.

### Example 37

### (R)-(2-benzylpiperazin-1-yl)(1-((5-methyl-2-phenyloxazol-4-yl)methyl)-1H-indazol-3-yl)methanone

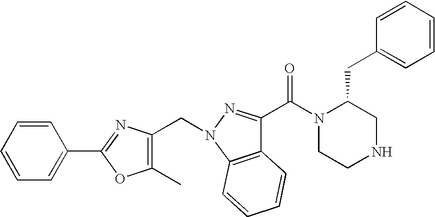

### 37A. 1-((5-methyl-2-phenyloxazol-4-yl)methyl)-1H-indazole-3-carboxylic acid

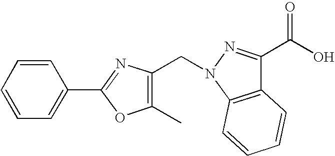

The title compound can be prepared by the nucleophilic displacement of 4-(chloromethyl)-5-methyl-2-phenyloxazole (Bioorg Med Chem Lett. 2004, 14, 6113-6116) with methyl-1H-indazole-3-carboxylate, using base (NaH or K₂CO₃) in DMF or THF and heating to 50-140° C. for 0.5 h to 3 d. The resulting ester can be saponified with a base (e.g. excess 1 M aq. NaOH in MeOH (0.1-0.3 M)) with heating.

### 37B. (R)-(2-benzylpiperazin-1-yl)(1-((5-methyl-2-phenyloxazol-4-yl)methyl)-1H-indazol-3-yl)methanone

The title compound was prepared by the coupling of 1-((5-methyl-2-phenyloxazol-4-yl)methyl)-1H-indazole-3-carboxylic acid to (R)-tert-butyl-3-benzylpiperazine-1-carboxylate as described in Scheme 2. ESI-MS: m/z 492 (M+H)⁺.

### Example 38

### (R)-(2-benzylpiperazin-1-yl)(1-tert-butyl-5-phenyl-1H-pyrazol-4-yl)methanone

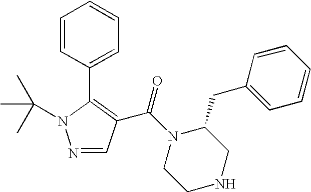

The title compound was prepared as described for Example 20. ESI-MS: m/z 403 (M+H)⁺.

### Example 39

### N-(2-(2-(2-((R)-2-benzylpiperazine-1-carbonyl)thiophen-3-yl)phenoxy)phenethyl)acetamide

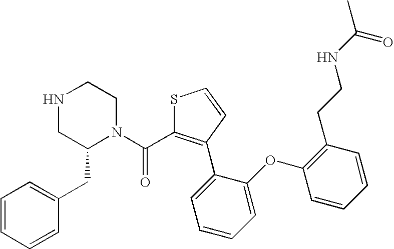

### 39A. ((R)-2,4-dibenzylpiperazin-1-yl)(3-(2-hydroxyphenyl)thiophen-2-yl)methanone

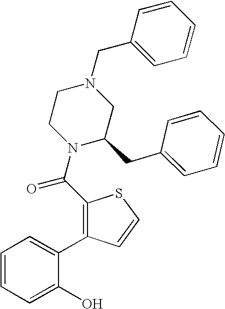

(R)-(3-bromothiophen-2-yl)(2,4-dibenzylpiperazin-1-yl)methanone (1.00 g, 0.220 mmol) and 2-hydroxyphenylboronic acid (363 mg, 0.263 mmol) were dissolved in DME (4 mL) and Na₂CO₃ (2M aq., 2.5 mL, 5.00 mmol) was added. The reaction mixture was purged with nitrogen and Pd(PPh₃)₄ (200 mg, 0.173 mmol) was added. The reaction mixture was stirred at 90° C. for 20 h, diluted with ethyl acetate (15 mL) and water (10 mL). The layers were separated and the water layer was extracted with ethyl acetate (5 mL). The combined organic layers were washed with water (10 mL), HCl (1N aq., 10 mL), NaHCO₃ (sat. aq., 10 mL), brine (10 mL), dried (MgSO₄), filtered and concentrated in vacuo. The crude product was purified using flash column chromatography on silica gel (90g SiO₂, hexanes-ethyl acetate-dichloromethane 4:4:1, 900 mL) to afford the title compound as an off-white solid (0.440 g, 43%). ESI-MS: m/z 469.4 (M+H)⁺.

### 39B. 2-(2-(2-((R)-2,4-dibenzylpiperazine-1-carbonyl)thiophen-3-yl)phenoxy)benzaldehyde

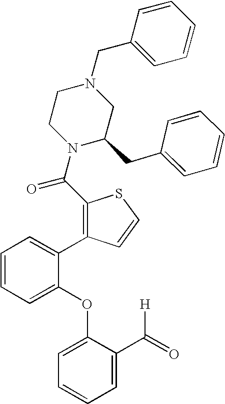

The title compound was prepared as described for Example 9-C, using 2-fluorobenzaldehyde instead of 2-chloro-4,6-dimethylnicotinonitrile. ESI-MS: m/z 573.4 (M+H)⁺.

### 39C. N-(2-(2-(2-((R)-2,4-dibenzylpiperazine-1-carbonyl)thiophen-3-yl)phenoxy)benzyl)acetamide

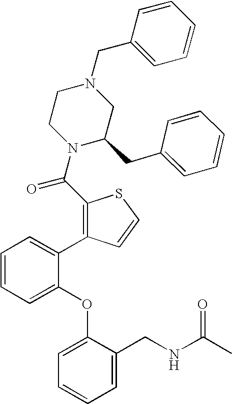

### 39D. N-(2-(2-(2-((R)-2-benzylpiperazine-1-carbonyl)thiophen-3-yl)phenoxy)phenethyl)acetamide

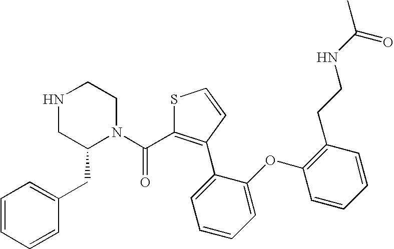

### Example 40

### N-((2-(2-(2-((R)-2-benzylpiperazine-1-carbonyl)thiophen-3-yl)phenoxy)pyridin-3-yl)methyl)acetamide

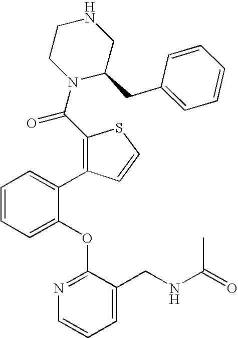

### 40A. (3R)-tert-butyl 4-(3-(2-(3-(acetamidomethyl)pyridin-2-yloxy)phenyl)thiophene-2-carbonyl)-3-benzylpiperazine-1-carboxylate

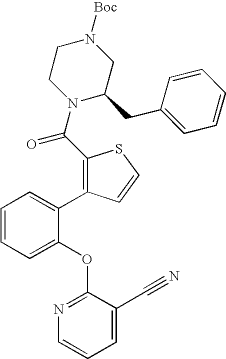

The title compound was prepared as described for Example 9-A-C, using 2-chloronicotinonitrile instead of 2-chloro-4,6-dimethylnicotinonitrile. ESI-MS: m/z 581.4 (M+H)⁺.

### 40B. (3R)-tert-butyl 4-(3-(2-(3-(acetamidomethyl)pyridin-2-yloxy)phenyl)thiophene-2-carbonyl)-3-benzylpiperazine-1-carboxylate

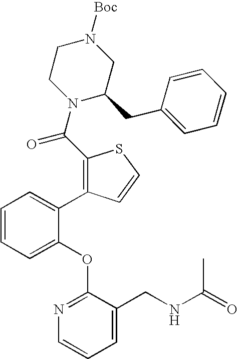

The title compound was prepared as described for Example 39A-C. ESI-MS: m/z 627.5 (M+H)⁺.

### 40C. N-((2-(2-(2-((R)-2-benzylpiperazine-1-carbonyl)thiophen-3-yl)phenoxy)pyridin-3-yl)methyl)acetamide

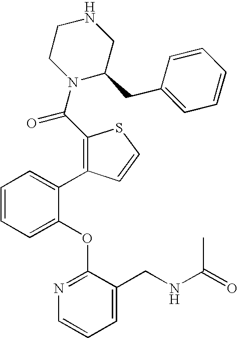

The title compound was prepared as described for Example 9-D. ESI-MS: m/z 527.4 (M+H)⁺.

### Example 41

### ((R)-2-benzylpiperazin-1-yl)(2-(2-(morpholinomethyl)phenyl)-thiophen-3-yl)methanone

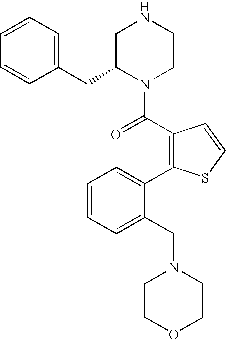

The title compound was prepared as described for Example 9 using 2-(morpholinomethyl)phenylboronic acid instead of 2-phenoxyphenylboronic acid. ESI-MS: m/z 462.4 (M+H)⁺.

### Example 42

### ((R)-2-benzylpiperazin-1-yl)(1-(2-phenoxyphenyl)-1H-pyrrol-2-yl)methanone

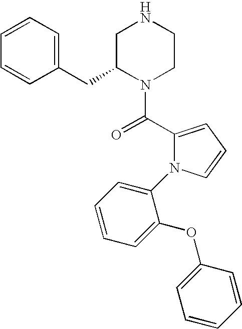

(R)-tert-butyl 3-benzyl-4-(1H-pyrrole-2-carbonyl)piperazine-1-carboxylate (25 mg, 0.06 mmol) was dissolved in dichloromethane (0.5 mL) and Cu(Oac)₂ (17 mg, 0.094 mmol) was added followed by 2-phenoxyphenylboronic acid (26.9 mg, 0.13 mmol) and pyridine (0.01 mL, 0.13 mmol). The reaction was allowed to stir at room temperature overnight. Excess dichloromethane (20 mL) was added and the solution was washed with water (25 mL) and brine (20 mL). The organics were collected, dried with sodium sulfate, and concentrated to an oil. The residue was taken up in dichloromethane (5 mL) and trifluoroacetic acid (2 mL) was added. The mixture was stirred at room temperature for 1 hour and then concentrated to an oil in vacuo. The residue was purified by HPLC (30-60% acetonitrile in water, TFA buffered) to afford the title compound as a white semisolid (27 mg, 66%). ESI-MS: m/z 438.4 (M+H)⁺.

### Example 43

### (R)-(2-benzylpiperazin-1-yl)(4-(2-phenoxyphenyl)thiophen-2-yl)methanone

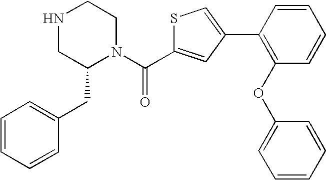

The title compound was prepared as described for Example 12. ESI-MS: m/z 455.3 (M+H)⁺.

### Example 44

### (R)-(2-benzylpiperazin-1-yl)(5-(2-phenoxyphenyl)thiophen-2-yl)methanone

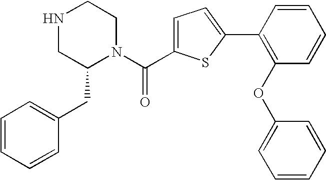

The title compound was prepared as described for Example 12. ESI-MS: m/z 455.3 (M+H)⁺.

### Example 45

### (R)-(2-benzylpiperazin-1-yl)(5-(2-phenoxyphenyl)furan-2-yl)methanone

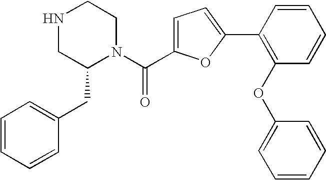

The title compound was prepared as described for Example 12. ESI-MS: m/z 439.4 (M+H)⁺.

### Example 46

### (R)-(2-benzylpiperazin-1-yl)(5-(biphenyl-2-yl)-1,3,4-oxadiazol-2-yl)methanone

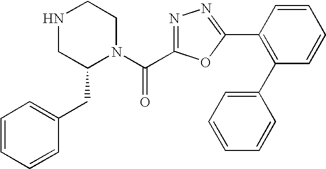

### 46A. 2-(Biphenyl-2-yl)-1,3,4-oxadiazole

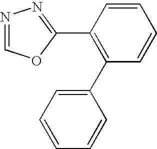

Biphenyl-2-carbohydrazide (1.00 g, 4.71 mmol) was suspended in triethylorthoformate (10 mL) and heated to 145° C. in a closed vial for 11 h. The reaction mixture was concentrated in vacuo at 80° C. and then in vacuum (0.2 mm Hg) at 80° C. to afford the title compound as a clear yellow oil (1.04 g, 99%).

### 46B. (R)-(2-benzylpiperazin-1-yl)(5-(biphenyl-2-yl)-1,3,4-oxadiazol-2-yl)methanone

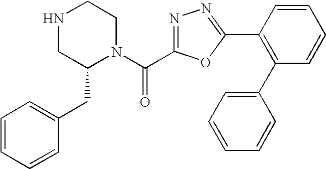

2-(Biphenyl-2-yl)-1,3,4-oxadiazole (260 mg, 1.17 mmol) was dissolved in THF, cooled to −78° C. under nitrogen atmosphere and treated dropwise with n-BuLi (1.6 M in hexanes, 0.73 mL, 1.17 mmol). The yellow solution was stirred for 1 h and CO₂ (crushed solid, ˜2 g) was added in one portion to the reaction mixture. The mixture was stirred at −78° C. for 2 h and a solution of (R)-tert-butyl 3-benzylpiperazine-1-carboxylate (332 mg, 1.20 mmol), EDCI (230 mg, 1.20 mmol), HOBt (180 mg, 1.18 mmol) in dichloromethane (2 mL) was added dropwise. The opaque reaction mixture was allowed to warm to room temperature, diluted with DMF (3 mL) and concentrated in vacuo until THF and dichloromethane were evaporated. The resulting DMF solution was stirred for 2 h, concentrated in vacuo at 90° C. and the residue dissolved in dichloromethane (3 mL) and treated with trifluoroacetic acid (3 mL). The reaction mixture was stirred overnight, concentrated in vacuo, diluted with water (3 mL) and brought to pH>13 with NaOH (5N). The mixture was extracted with EtOAc (5 3 mL), and the combined organic extracts were concentrated under reduced pressure. The crude product was purified by HPCL (30-45% acetonitrile in water, buffered with TFA) to afford the title compounds as a white solid (TFA salt, 0.108 g, 17%). ESI-MS: m/z 425.4 (M+H)⁺.

### BIOLOGICAL EXAMPLES

### Example A

### Expression of Preprorenin and Purification of Prorenin

The sequence of human wild-type renin is known in the art; see, Imai, T. et al., Proc. Natl. Acad. Sci. USA 1983, 80, 7105-7409. It is noted that the fragment of the renin protein useful for the assay comprises amino acid residues 67-406 of human renin (active Renin). To prepare active Renin, a fragment longer than active Renin, a preprorenin (e.g., comprising residues 1-406), may be expressed and from which a prorenin (e.g., comprising residues 23-406) may be recovered. The prorenin may later be cleaved to obtain active Renin.

Expression of human preprorenin (residues 1-406) can be conducted using a FreeStyle 293 Expression System (Invitrogen Corp.), wherein the plasmid DNA for human prorenin expression (pcDNA3.1(+)/hREN) is used to conduct transient expression in FreeStyle 293-F cells. After transfection of the plasmid DNA, the cells are subjected to shaking at 37° C., 8% CO₂ and 125 rpm for 3 days.

The prorenin protein is then accumulated and purified by salting out. Powdered ammonium sulfate is added to the culture medium and dissolved to make a 40% saturation of the salt. The resulting precipitate can be collected by centrifugation and discarded. Ammonium sulfate is added to the remaining solution and dissolved to make an 80% saturation of salt. The resulting precipitate can be collected by, for example, centrifugation. The prorenin protein is recovered by dissolving the precipitate in buffer.

The concentrated liquid is subjected to gel filtration chromatography using, for example, HiLoad 16/60 Superdex 200 pg (Amersham Biosciences, Inc.) equilibrated with 20 mM Tris-hydrochloric acid (pH 8.0) containing 0.15 M sodium chloride, at a flow rate of 1.4 ml/min, to obtain 3.6 mg of purified prorenin (residues 24-406).

### Example B

### Purification of Active Renin

To 3.6 mg of prorenin (residues 24-406, as prepared in Example A) dissolved in 5.2 ml of 0.1 M Tris-hydrochloric acid (pH 8.0), is added 12 pg of trypsin (Roche Diagnostics Corp.), and the mixture is allowed to react at 28° C. for 55 minutes to carry out activation of Renin. After the reaction, 0.4 ml of immobilized trypsin inhibitor (Pierce Biotechnology, Inc.) is added to remove the trypsin used in the activation, by adsorption. The reaction liquid containing the active renin is concentrated using Vivaspin 20 (molecular weight of the fraction 10,000; Vivascience, Inc.), and diluted with 20 mM Tris-hydrochloric acid (pH 8.0). The diluted liquid is fed to a TSKgel DEAE-5 PW column (7.5 mm I.D.×75 mm, Tosoh Corp.) equilibrated with 20 mM Tris-hydrochloric acid (pH 8.0) at a flow rate of 1 ml/min to adsorb the active renin (residues 67-406). The column is washed with the buffer solution used for the equilibration, and then elution is carried out by means of a linear concentration gradient of sodium chloride from 0 M to 0.3 M, to obtain 1.5 mg of purified active renin (residues 67-406).

### Example C

### Establishment of Renin Expressing Vector

A plasmid DNA to express human renin in HEK293 cells can be prepared as follows. PCR is carried out using human renal cDNA (Clontech Laboratories, Inc., Marathon Ready cDNA) as the template and using two synthetic DNAs (5′-AAGCTTATGGATGGATGGAGA-3′ (SEQ ID NO: 1) and 5′-GGATCCTCAGCGGGCCAAGGC-3′ (SEQ ID NO: 2)), and the obtained fragment is cloned using TOPO TA Cloning Kit (Invitrogen Corp.). The obtained fragment is subcloned into pcDNA3.1 (+) that has been cleaved by HindIII and BamHI, to obtain a plasmid DNA for human preprorenin expression (pcDNA3.1(+)/hREN).

### Example D

### Assaying the In Vitro Enzymatic Activity of Renin Inhibitors

Solutions of test compounds in varying concentrations (≦2 mM final concentration) are prepared in dimethyl sulfoxide (DMSO) and then diluted into assay buffer comprising 50 mM Hepes, 1 mM EDTA, 1 mM DTT, 0.1 mg/ml BSA, 0.01% Brij35, pH 7.4. Alternatively, the assay can be performed with a high BSA concentration, wherein the buffer contains an additional 2% BSA.

Recombinant human renin (3 nM final concentration) is added to the dilutions and pre-incubated with the compounds for 10 minutes at 37° C. As described in Examples A-C above, human renin can be obtained by expressing preprorenin (residue 1-406) in mammalian cells, treating the prorenin (residues 24-406) contained in the culture supernatant with trypsin, and isolate the active form (residues 67-406). After pre-incubation, the reaction is initiated with 1 μM of substrate QXL520-γ-Abu-Ile-His-Pro-Phe-His-Leu-Val-Ile-His-Thr-Lys (HiLyteFluo488)-Arg-OH (Anaspec, San Jose, Calif.). The final DMSO in the assay is 5%. The total volume of the reaction mixture is 20 μL, which can be placed on Greiner 384-well small volume plates.

Renin activity may be determined via fluorescence (excitation λ=485 nm; emission λ=538 nm), e.g., on a Molecular Devices SPECTROmax GEMINI XPS. The fluorescence intensity is determined upon the addition of substrate and determined again after incubation at 37° C. for one hour. The fluorescence intensity of a blank (no inhibition) using vehicle alone is also determined. Renin activity is linearly proportional to the change in fluorescence observed (final−initial).

The percent inhibition of renin at a given compound concentration is defined as:

100%×[1−(F_{compound}/F_{blank})]

where F_{compound} is the observed fluorescence at a given concentration of test compound and F_{blank} is the observed fluorescence in the presence of vehicle alone.

The pIC₅₀ value (negative log of the molar concentration of the compound that produces 50% inhibition) of a test compound is calculated by non-linear least squares curve fitting of the equation:

Percent Inhibition=100%/(1+(10^{-pIC50}/10^{log [I]}))

to percent inhibition versus compound concentration. The 50% inhibitory concentration (IC₅₀) of a test compound is calculated by raising 10 to the negative pIC₅₀ (10^{-pIC50}).

IC₅₀ values for selected compounds of the present invention are given in Table 1.

TABLE 1
IC₅₀ of Exemplified Compounds Against Renin

| EXAMPLE | IC₅₀ (μM) |
|---|---|
| 1 | <1 |
| 2 | >100 |
| 3 | <1 |
| 4 | 1-50 |
| 5 | 1-50 |
| 6 | 1-50 |
| 7 | 1-50 |
| 8 | 50-100 |
| 9 | 1-50 |
| 10 | 1-50 |
| 11 | 1-50 |
| 12 | <1 |
| 13 | 1-50 |
| 14 | 1-50 |
| 15 | 1-50 |
| 16 | 1-50 |
| 17 | <1 |
| 18 | 1-50 |
| 19 | 1-50 |
| 20 | 1-50 |
| 21 | 1-50 |
| 22 | 1-50 |
| 23 | 1-50 |
| 24 | 1-50 |
| 25 | 50-100 |
| 26 | >100 |
| 27 | 1-50 |
| 28 | >100 |
| 29 | 50-100 |
| 30 | 1-50 |
| 31 | 1-50 |
| 32 | 1-50 |
| 33 | 1-50 |
| 34 | 1-50 |
| 35 | 1-50 |
| 36 | 1-50 |
| 37 | 1-50 |
| 38 | 1-50 |
| 39 | <1 |
| 40 | 1-50 |
| 41 | 1-50 |
| 42 | 1-50 |
| 43 | 1-50 |
| 44 | 1-50 |
| 45 | 1-50 |
| 46 | 1-50 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A compound of a formula selected from the group consisting of: 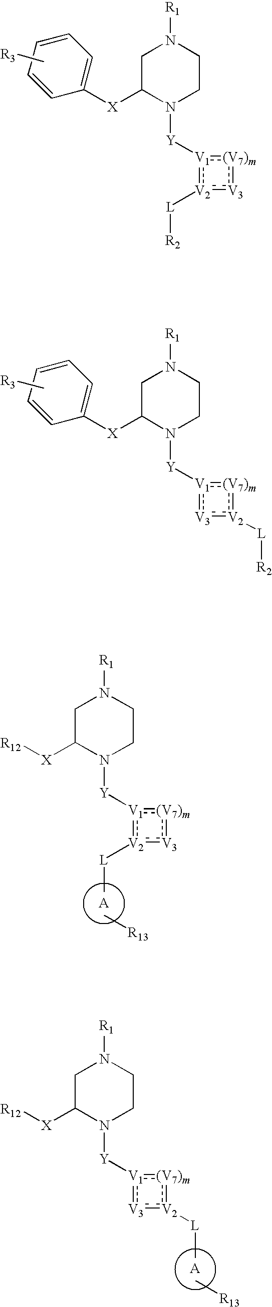 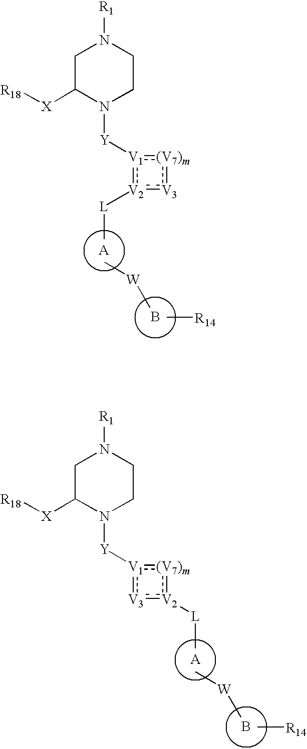
wherein
m is selected from the group consisting of 0, 1, 2, 3, and 4;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
L is absent or is a linker providing 1, 2 or 3 atom separation between V₂ and R₂, wherein the atoms of the linker providing the separation are selected from the group consisting of carbon, oxygen, nitrogen, and sulfur atoms;
V₁ and V₂ are each independently selected from the group consisting of —CR₂₂— and —N—;
V₃ is selected from the group consisting of —CH₂—, —CH═, —NH—, —N═, —O— and —S—;
each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen;
W is absent or is selected from the group consisting of —CR₁₅R₁₆—, —NR₁₇—, —O— and —S—;
X is —(CR₄R₅)ₙ—, where n is selected from the group consisting of 1 and 2;
Y is selected from the group consisting of —C(O)— and —S(O)₂—;
R₁ is selected from the group consisting of hydrogen and a substituent convertible in vivo to hydrogen;
R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted;
R₃ is selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, aminocarbonyloxy and carbonylalkoxy, each substituted or unsubstituted;
R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R₁₂ is a substituted or unsubstituted phenyl or (C₄₋₇)heteroaryl;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond;
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and
R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;
or any two R₁₉, R₂₀ and R₂₁ are taken together to form a 3, 4, 5, 6 or 7 membered ring; and
wherein the compound includes any hydrate, solvate, tautomer, enantiomer, and pharmaceutically acceptable salt form of the compound.

2. The compound of claim 1 having a formula selected from the group consisting of: 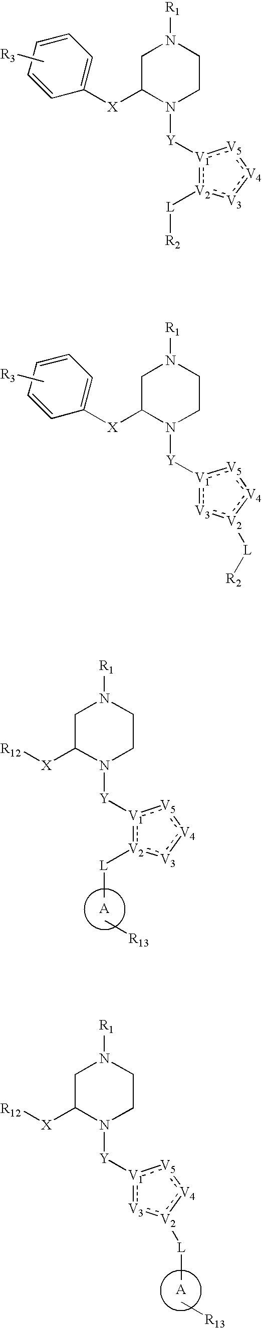 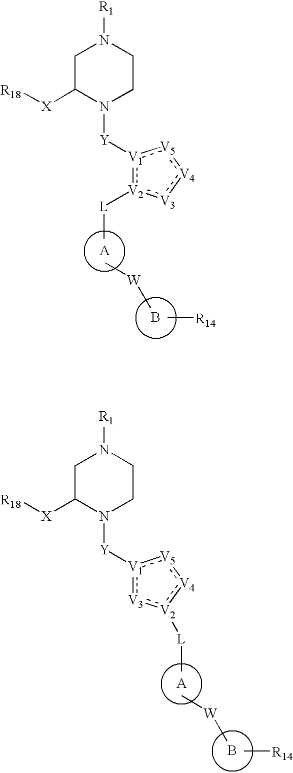
wherein
V₄ and V₅ are each independently selected from the group consisting of —CR₁₉R₂₀, —NR₂₁—, —O— and —S—, and at least one of V₄ and V₅ is selected from the group consisting of —CH₂—, —CH═, —NH—, —N═, —O— and —S—.

3. The compound of claim 1 having a formula selected from the group consisting of: 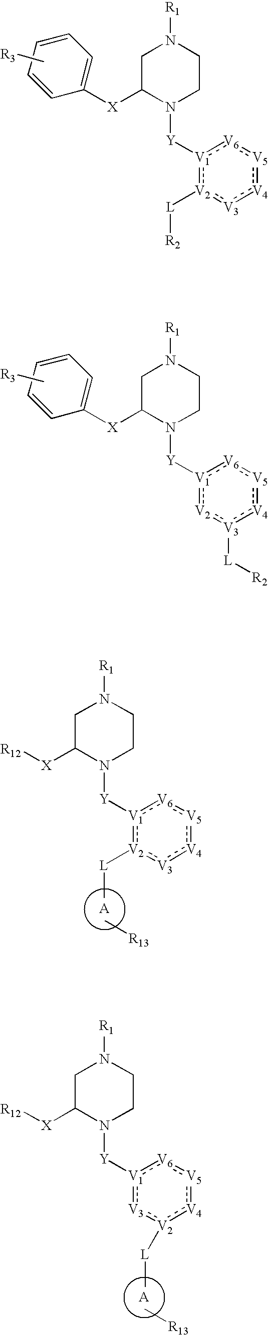 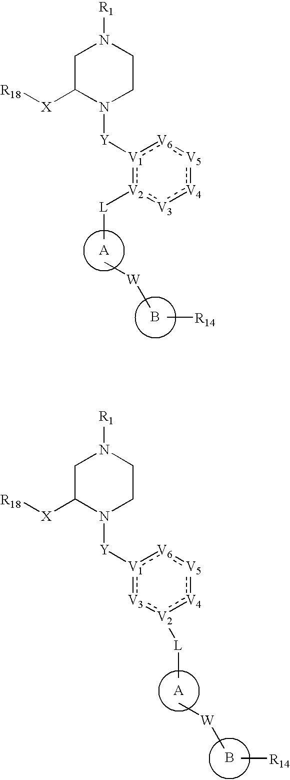
wherein
V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—.

4. The compound according to claim 1, wherein X is selected from the group consisting of —CH₂— and —CH₂CH₂—.

5. The compound according to claim 1, wherein R₄ is selected from the group consisting of hydrogen and halo.

6. The compound according to claim 1, wherein R₅ is selected from the group consisting of hydrogen and halo.

7. The compound according to claim 1, wherein R₁ is hydrogen.

8. The compound according to claim 1, wherein R₁ is methyl or ethyl.

9. The compound according to claim 1, wherein R₁₂ is a substituted or unsubstituted phenyl.

10. The compound according to claim 1, wherein R₃ is selected from the group consisting of 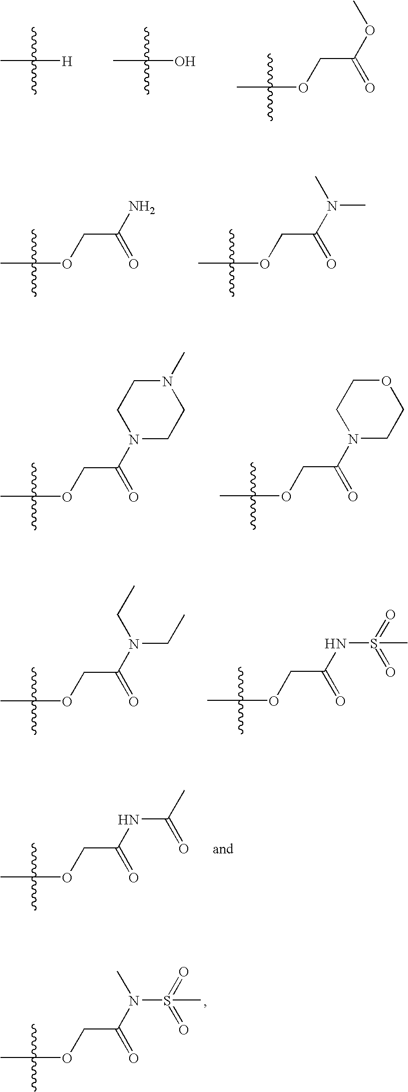
each substituted or unsubstituted.

11. The compound according to claim 1, wherein R₂ is selected from the group consisting of phenyl and hetero(C₁₋₅)aryl, each unsubstituted or substituted with one or more substituents.

12. The compound according to claim 11, wherein the substituents on R₂ are each independently selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀)alkyl, each substituted or unsubstituted.

13. The compound according to claim 11, wherein R₂ is substituted with a substituent selected from the group consisting of 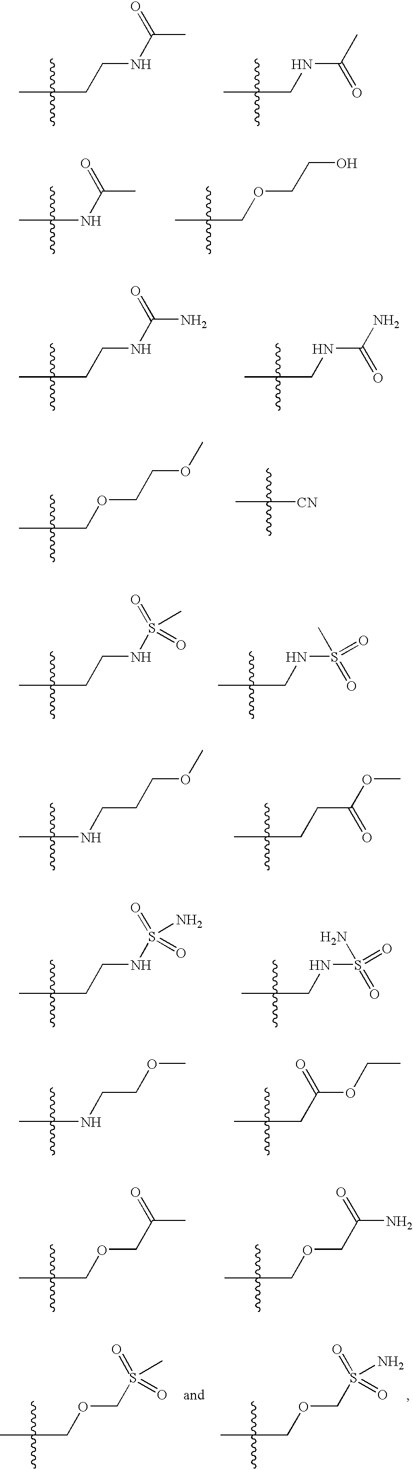
each substituted or unsubstituted.

14. The compound according to claim 1, wherein ring A is selected from the group consisting of phenyl and hetero(C₁₋₅)aryl, each substituted or unsubstituted.

15. The compound according to claim 1, wherein R₁₃ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl and cycloalkoxy(C₃₋₆)carbonylalkyl(C₁₋₁₀-)alkyl, each substituted or unsubstituted.

16. The compound according to claim 1, wherein R₁₃ is selected from the group consisting of 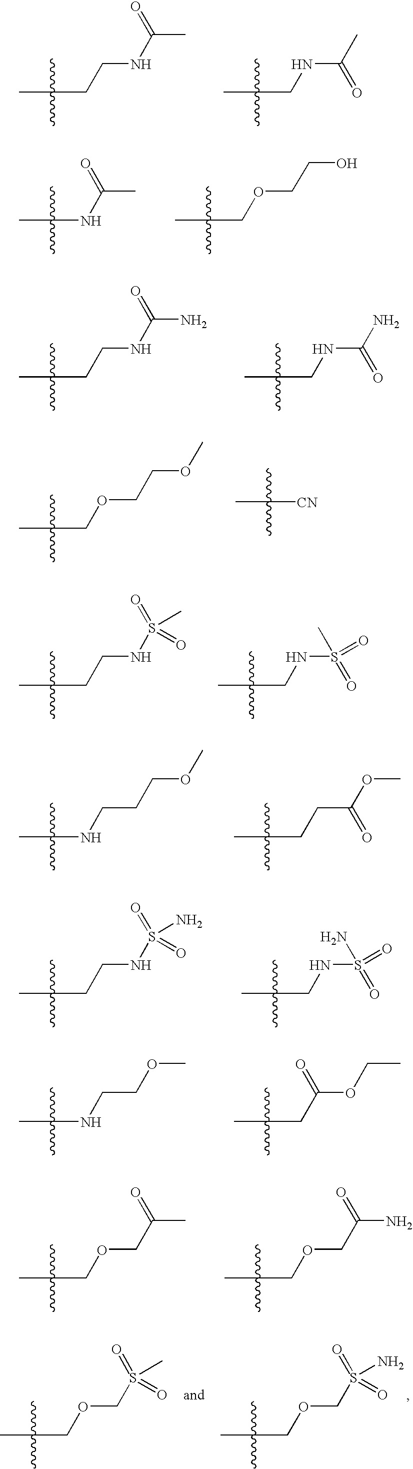
each substituted or unsubstituted.

17. The compound according to claim 1, wherein W is selected from the group consisting of —CR₁₅R₁₆—, —NH— and —O—, wherein R₁₅ and R₁₆ are each independently selected from the group consisting of hydrogen, halo, hydroxyl and substituted or unsubstituted (C₁₋₃)alkyl.

18. The compound according to claim 1, wherein R₁₅ is selected from the group consisting of hydrogen, hydroxyl, halo and substituted or unsubstituted (C₁₋₃)alkyl.

19. The compound according to claim 1, wherein R₁₆ is selected from the group consisting of hydrogen, hydroxyl, halo, and substituted or unsubstituted (C₁₋₃)alkyl.

20. The compound according to claim 1, wherein W is selected from the group consisting of —NR₁₇—, where R₁₇ is selected from the group consisting of 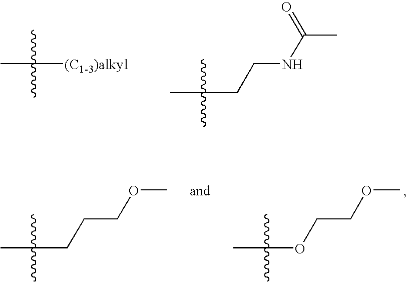
each substituted or unsubstituted.

21. The compound according to claim 1, wherein R₁₈ is selected from the group consisting of (C₁₋₆)alkyl, (C₃₋₇)cycloalkyl, (C₄₋₇)aryl and hetero(C₁₋₅)aryl, each substituted or unsubstituted.

22. The compound according to claim 1, wherein ring B is selected from the group consisting of phenyl and hetero(C₁₋₁₀)aryl, each substituted or unsubstituted.

23. The compound according to claim 1, wherein R₁₄ is selected from the group consisting of halo, (C₁₋₃)alkyl, hydroxy(C₁₋₃)alkyl, hydroxycarbonyl (C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl; hydroxycarbonyl(C₁₋₁₀)alkyl, alkyl(C₁₋₃)aminocarbonylalkyl(C₁₋₁₀)alkyl, alkoxy(C₁₋₃)carbonylalkyl(C₁₋₁₀)alkyl, cycloalkoxy(C₃₋₆)carbonylalkyl(C₁₋₁₀)alkyl, amido(C₁₋₁₀)alkyl, alkoxyalkoxy(C₁₋₁₀)alkyl, alkoxy(C₁₋₁₀)alkyl and alkoxyalkoxy, each substituted or unsubstituted.

24. The compound according to claim 1, wherein R₁₄ is selected from the group consisting of 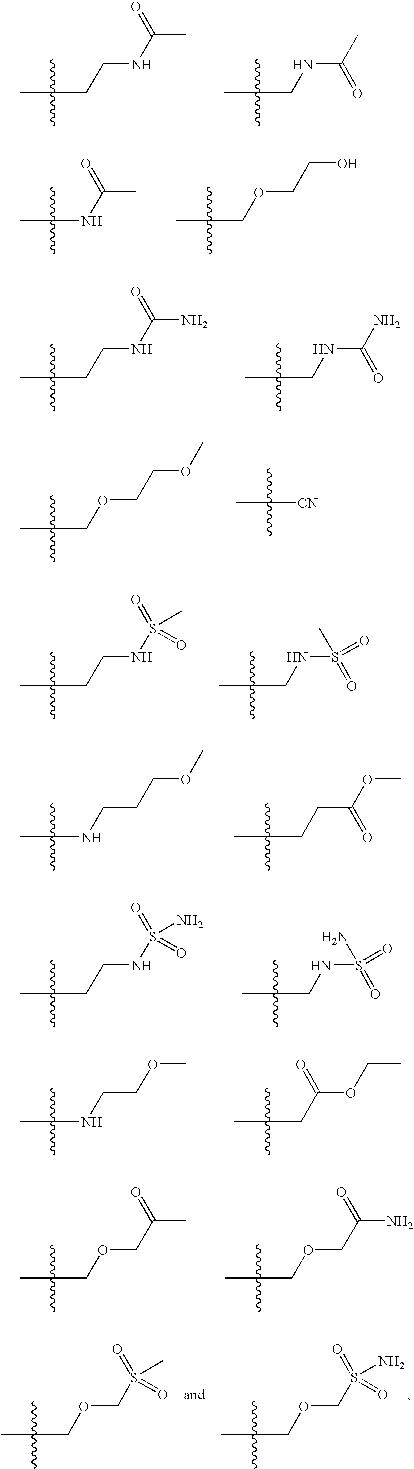
each substituted or unsubstituted.

25. The compound according to claim 1, wherein V₁ is —C═.

26. The compound according to claim 1, wherein V₂ is —C═.

27. The compound according to claim 1, wherein V₃ is selected from the group consisting of —CH═ and —NH═.

28. The compound according to claim 1, wherein V₁, V₂ and V₃ are each independently selected from the group consisting of —C═, —N—, —CH═, and —N═.

29. The compound according to claim 2, wherein V₄ is selected from the group consisting of —CH═ and —N═.

30. The compound according to claim 3, wherein V₄ is selected from the group consisting of —CH═ and —N═.

31. The compound according to claim 2, wherein V₅ is selected from the group consisting of —CH═ and —N═

32. The compound according to claim 3, wherein V₅ is selected from the group consisting of —CH═ and —N═.

33. The compound according to claim 3, wherein V₆ is selected from the group consisting of —CH═ and —N═.

34. The compound according to claim 1, wherein R₁₉ is selected from the group consisting of hydrogen, halo, oxy, hydroxyl, (C₁₋₃)alkyl and alkoxy, each substituted or unsubstituted.

35. The compound according to claim 1, wherein R₂₀ is selected from the group consisting of hydrogen, halo, oxy, hydroxyl, (C₁₋₃)alkyl and alkoxy, each substituted or unsubstituted.

36. The compound according to claim 1, wherein R₂₁ is selected from the group consisting of hydrogen and substituted or unsubstituted (C₁₋₃)alkyl.

37. The compound according to claim 1, wherein L is absent.

38. The compound according to claim 1, wherein Y is —C(O)—.

39. A compound selected from the group consisting of:
((R)-2-benzylpiperazin-1-yl)(3-(2-phenoxyphenyl)thiophen-2-yl)methanone;
((S)-2-benzylpiperazin-1-yl)(2-phenylcyclopropyl)methanone;
(R)-(2-benzylpiperazin-1-yl)(3′,4′-dimethylbiphenyl-2-yl)methanone;
(R)-(2-benzylpiperazin-1-yl)(biphenyl-2-yl)methanone;
((R)-2-benzylpiperazin-1-yl)(2′-chlorobiphenyl-2-yl)methanone;
(R)-(2-benzylpiperazin-1-yl)(1-phenyl-1H-pyrazol-5-yl)methanone;
(R)-(2-benzylpiperazin-1-yl)(5-phenyloxazol-4-yl)methanone;
((R)-2-benzylpiperazin-1-yl)(1-(2-(trifluoromethyl)phenyl)-1H-imidazol-2-yl)methanone;
2-(2-(2-((R)-2-benzylpiperazine-1-carbonyl)thiophen-3-yl)phenoxy)-4,6-dimethylnicotinonitrile;
2-(2-(2-((R)-2-benzylpiperazine-1-carbonyl)thiophen-3-yl)phenoxy)benzonitrile;
2-(2-(2-((R)-2-benzylpiperazine-1-carbonyl)thiophen-3-yl)phenoxy)nicotinonitrile;
((R)-2-benzylpiperazin-1-yl)(2-(2-phenoxyphenyl)thiophen-3-yl)methanone;
((R)-2-benzylpiperazin-1-yl)(1,3-dimethyl-4-(2-phenoxyphenyl)-1H-pyrazol-5-yl)methanone;
((R)-2-benzylpiperazin-1-yl)(1-ethyl-3-methyl-4-(2-phenoxyphenyl)-1H-pyrazol-5-yl)methanone;
((R)-2-benzylpiperazin-1-yl)(5-fluoro-2′-phenoxybiphenyl-2-yl)methanone;
((R)-2-benzylpiperazin-1-yl)(4-fluoro-2′-phenoxybiphenyl-2-yl)methanone;
((R)-2-benzylpiperazin-1-yl)(1-methyl-4-(2-phenoxyphenyl)-1H-pyrazol-3-yl)methanone;
(R)-(3-(1-benzyl-1H-pyrazol-4-yl)thiophen-2-yl)(2-benzylpiperazin-1-yl)methanone;
((R)-2-benzylpiperazin-1-yl)(2-(biphenyl-2-yl)-thiophen-3-yl)-methanone;
(R)-2-(2-(2-benzylpiperazine-1-carbonyl)phenylthio)benzonitrile;
(R)-(2-benzylphenyl)(2-benzylpiperazin-1-yl)methanone;
(R)-(2-benzoylphenyl)(2-benzylpiperazin-1-yl)methanone;
(R)-(2-(benzyloxy)phenyl)(2-benzylpiperazin-1-yl)methanone;
1-benzhydryl-4-((R)-2-benzylpiperazine-1-carbonyl)pyrrolidin-2-one;
(R)-(2-benzylpiperazin-1-yl)(2-(4-methoxybenzoyl)phenyl)methanone;
((R)-2-benzylpiperazin-1-yl)(1-(naphthalen-1-yl)-1H-imidazol-5-yl)methanone;
((R)-2-benzylpiperazin-1-yl)(1-(biphenyl-2-yl)-1H-imidazol-5-yl)methanone;
((R)-2-benzylpiperazin-1-yl)(2-(3,5-dimethyl-1H-pyrazol-4-yl)phenyl)methanone;
(R)-(2-benzylpiperazin-1-yl)(3-methyl-5-phenylisoxazol-4-yl)methanone;
(R)-(2-benzylpiperazin-1-yl)(5-phenyl-2-(pyridin-3-yl)thiazol-4-yl)methanone;
(R)-(2-benzylpiperazin-1-yl)(4′-fluorobiphenyl-2-yl)methanone;
(R)-(2-(benzo[d]thiazol-2-yl)phenyl)(2-benzylpiperazin-1-yl)methanone;
((R)-2-benzylpiperazin-1-yl)(3-(2-chlorophenyl)-5-cyclopropylisoxazol-4-yl)methanone;
(R)-(6-benzyl-2-methyl-6H-thieno[2,3-b]pyrrol-5-yl)(2-benzylpiperazin-1-yl)methanone;
(R)-(2-benzylpiperazin-1-yl)(2,2-dimethyl-2,3-dihydrobenzofuran-7-yl)methanone;
(R)-(2-benzylpiperazin-1-yl)(4-(4-chlorophenyl)thiophen-2-yl)methanone;
(R)-(2-benzylpiperazin-1-yl)(1-((5-methyl-2-phenyloxazol-4-yl)methyl)-1H-indazol-3-yl)methanone;
(R)-(2-benzylpiperazin-1-yl)(1-tert-butyl-5-phenyl-1H-pyrazol-4-yl)methanone;
N-(2-(2-(2-((R)-2-benzylpiperazine-1-carbonyl)thiophen-3-yl)phenoxy)phenethyl)acetamide;
N-((2-(2-(2-((R)-2-benzylpiperazine-1-carbonyl)thiophen-3-yl)phenoxy)pyridin-3-yl)methyl)acetamide;
((R)-2-benzylpiperazin-1-yl)(2-(2-(morpholinomethyl)phenyl)-thiophen-3-yl)methanone;
((R)-2-benzylpiperazin-1-yl)(1-(2-phenoxyphenyl)-1H-pyrrol-2-yl)methanone;
(R)-(2-benzylpiperazin-1-yl)(4-(2-phenoxyphenyl)thiophen-2-yl)methanone;
(R)-(2-benzylpiperazin-1-yl)(5-(2-phenoxyphenyl)thiophen-2-yl)methanone;
(R)-(2-benzylpiperazin-1-yl)(5-(2-phenoxyphenyl)furan-2-yl)methanone; and
(R)-(2-benzylpiperazin-1-yl)(5-(biphenyl-2-yl)-1,3,4-oxadiazol-2-yl)methanone.

40. The compound according to claim 1, wherein the compound is in the form of a pharmaceutically acceptable salt.

41. The compound according to claim 1, wherein the compound is present in a mixture of stereoisomers.

42. The compound according to claim 1, wherein the compound comprises a single stereoisomer.

43. A pharmaceutical composition comprising as an active ingredient a compound according to claim 1.

44. The pharmaceutical composition according to claim 43, wherein the composition is a solid formulation adapted for oral administration.

45. The pharmaceutical composition according to claim 43, wherein the composition is a liquid formulation adapted for oral administration.

46. The pharmaceutical composition according to claim 43, wherein the composition is a tablet.

47. The pharmaceutical composition according to claim 43, wherein the composition is a liquid formulation adapted for parenteral administration.

48. A pharmaceutical composition comprising a compound according to claim 1, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

49. A kit comprising:
a compound according to claim 1; and
instructions which comprise one or more forms of information selected from the group consisting of indicating a disease state for which the compound is to be administered, storage information for the compound, dosing information and instructions regarding how to administer the compound.

50. The kit of claim 49, wherein the kit comprises the compound in a multiple dose form.

51. An article of manufacture comprising:
a compound according to claim 1; and
packaging materials.

52. The article of manufacture of claim 51, wherein the packaging material comprises a container for housing the compound.

53. The article of manufacture of claim 52, wherein the container comprises a label indicating one or more members of the group consisting of a disease state for which the compound is to be administered, storage information, dosing information and/or instructions regarding how to administer the compound.

54. The article of manufacture of claim 51, wherein the article of manufacture comprises the compound in a multiple dose form.

55. A therapeutic method comprising administering a compound according to claim 1 to a subject.

56. A method of inhibiting renin comprising contacting renin with a compound according to claim 1.

57. A method of inhibiting renin comprising causing a compound according to claim 1 to be present in a subject in order to inhibit renin in vivo.

58. A method of inhibiting renin comprising administering a first compound to a subject that is converted in vivo to a second compound wherein the second compound inhibits renin in vivo, the second compound being a compound according to claim 1.

59. A method of treating a disease state for which renin possesses activity that contributes to the pathology and/or symptomology of the disease state, the method comprising causing a compound according to claim 1 to be present in a subject in a therapeutically effective amount for the disease state.

60. The method according to claim 59, wherein the disease state is selected from the group consisting of cardiovascular disease, hypertension, congestive heart failure, myocardial infarction, renal protection, inflammation, neurological diseases and cancer.

61. A method of treating a disease state for which renin possesses activity that contributes to the pathology and/or symptomology of the disease state, the method comprising administering a compound according to claim 1 to a subject.

62. The method according to claim 61, wherein the disease state is selected from the group consisting of cardiovascular disease, hypertension, congestive heart failure, myocardial infarction, renal protection, inflammation, neurological diseases and cancer.

63. A method of treating a disease state for which renin possesses activity that contributes to the pathology and/or symptomology of the disease state, the method comprising administering a first compound to a subject that is converted in vivo to a second compound wherein the second compound inhibits renin in vivo, the second compound being a compound according to claim 1.

64. The method according to claim 63, wherein the disease state is selected from the group consisting of cardiovascular disease, hypertension, congestive heart failure, myocardial infarction, renal protection, inflammation, neurological diseases and cancer.

65. A method comprising:
coupling a compound having the formula 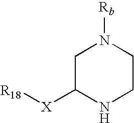
to a compound of the formula R′—Y-J,
under conditions that form a reaction product of the formula 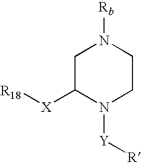
wherein
J is a leaving group;
X is —(CR₄R₅)ₙ—, where n is selected from the group consisting of 1 and 2, and R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
Y is selected from the group consisting of —C(O)— and —S(O)₂—;
R_{b} is selected from the group consisting of R₁, benzyl and Boc, where R₁ is selected from the group consisting of hydrogen and a substituent convertible in vivo to hydrogen;
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
R′ is selected from the group consisting of 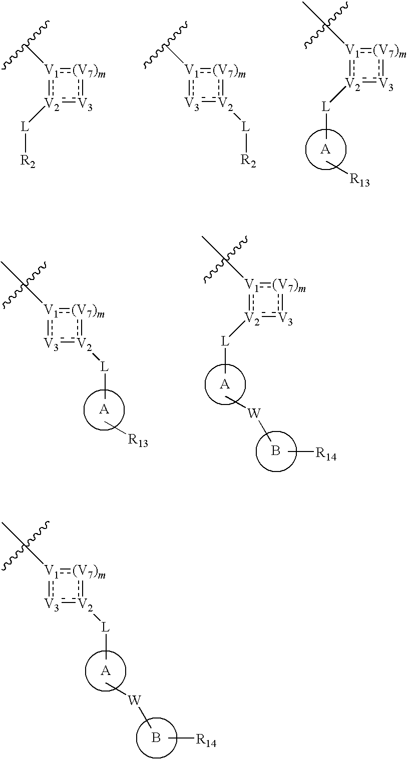
where
m is selected from the group consisting of 0, 1, 2, 3, and 4;
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted,
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted,
V₁ and V₂ are each independently selected from the group consisting of —CR₂₂— and —N—,
V₃ is selected from the group consisting of —CH₂—, —CH═, —NH—, —N═, —O— and —S—,
each V₇ is independently selected from the group consisting of —CR₁₉R₂₀—, —NR₂₁—, —O— and —S—, with the proviso that when m is 2 or 3, no two adjacent V₇ are both substituted with a substituent other than hydrogen,
L is absent or is a linker providing 1, 2 or 3 atom separation between ring V and R₂ or ring A, wherein the atoms of the linker providing the separation are selected from the group consisting of carbon, oxygen, nitrogen, and sulfur atoms,
W is absent or is selected from the group consisting of —CR₁₅R₁₆—, —NR₁₇—, —O— and —S—,
R₂ is selected from the group consisting of (C₃₋₇)cycloalkyl, hetero(C₃₋₇)cycloalkyl, (C₄₋₇)aryl and hetero(C₁₋₆)aryl, each substituted or unsubstituted,
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted,
R₁₄ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl, hetero(C₄₋₁₂)bicycloaryl, amidoalkyl, alkoxyalkoxyalkyl, alkoxyalkyl and alkoxyalkoxy, each substituted or unsubstituted,
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond, and
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond
R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and
R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;
or any two R₁₉, R₂₀ and R₂₁ are taken together to form a 3, 4, 5, 6 or 7 membered ring; and
wherein the reaction product includes any hydrate, solvate, tautomer, enantiomer, and pharmaceutically acceptable salt form of the reaction product.

66. The method according to claim 65, wherein Y is —C(O)—.

67. The method according to claim 65, wherein L is absent, or is selected from the group consisting of —S—, —C(O)— and —CH₂—.

68. The method according to claim 65, wherein L is absent.

69. The method according to claim 65, wherein W is absent, or is selected from the group consisting of —CR₁₅R₁₆—, —NH— and —O—, where R₁₅ and R₁₆ are each independently selected from the group consisting of hydrogen, halo, hydroxyl and substituted and unsubstituted (C₁₋₃)alkyl.

70. The method according to claim 65, wherein X is selected from the group consisting of —CH₂— and —CH₂CH₂—.

71. The method according to claim 65, wherein R₁₈ is an unsubstituted phenyl

72. The method according to claim 65, wherein R₁₈ is a substituted phenyl and the substituent is selected from the group consisting of 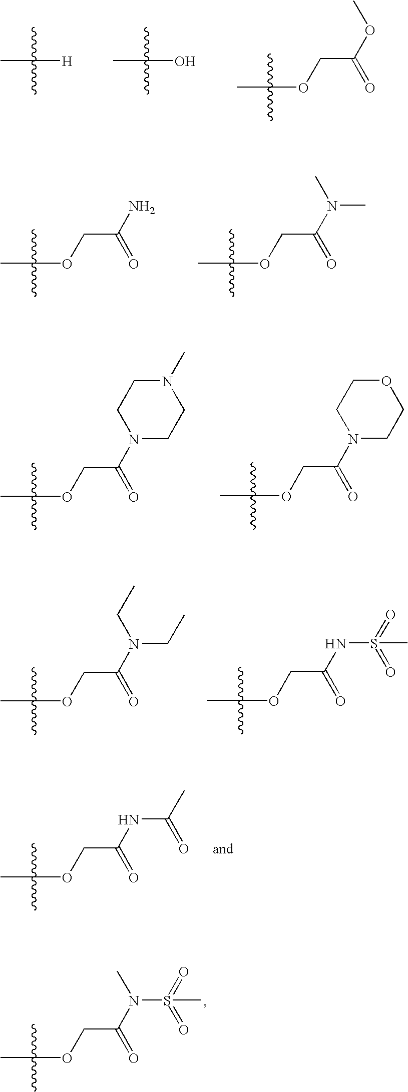
each substituted or unsubstituted.

73. The method according to claim 65 further comprising:
coupling a compound of the formula 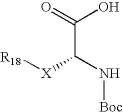
to another compound of the formula 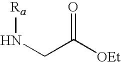
under conditions that form a compound having the formula 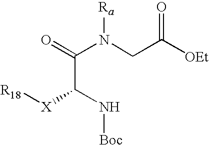
cyclizing the compound immediately above to form a compound having the formula 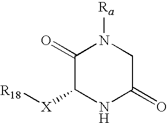
reducing the compound immediately above to a product having the formula 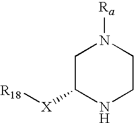
wherein
Rₐ is R₁ or benzyl.

74. A method comprising
coupling a compound having the formula 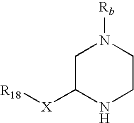
to a compound of a formula 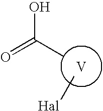
under conditions that form a compound of the formula: 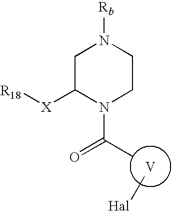
wherein
ring V is a 3, 4, 5, 6, or 7 membered, saturated, unsaturated, or aromatic carbocycle or heterocycle, substituted or unsubstituted;
Hal is selected from the group consisting of —Br, —I, —OTf, and —OMs;
X is —(CR₄R₅)ₙ—, where n is selected from the group consisting of 1 and 2, and R₄ and R₅ are each independently selected from the group consisting of hydrogen, halo, thio, oxy, hydroxyl and (C₁₋₁₀)alkyl, each substituted or unsubstituted, or R₄ and R₅ are taken together to form a ring;
R_{b} is selected from the group consisting of R₁, benzyl and a Boc group, and R₁ is selected from the group consisting of hydrogen and a substituent convertible in vivo to hydrogen; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted; and
the compound formed includes any hydrate, solvate, tautomer, enantiomer, and pharmaceutically acceptable salt form of the reaction product.

75. The method according to claim 74 further comprising
reacting the compound of the formula 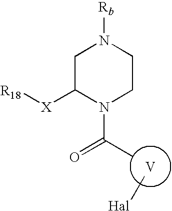
with a boronic acid derivative selected from the group consisting of: 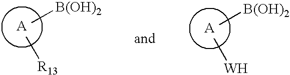
under conditions that form a compound selected from the group consisting of: 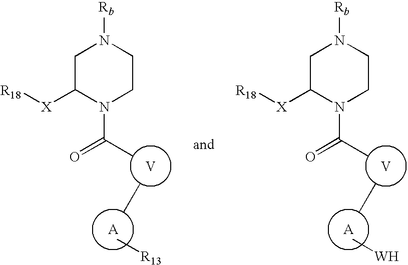
wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
W is absent or is selected from the group consisting of —CR₁₅R₁₆—, —NR₁₇, —O— and —S—;
R₁₃ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond; and
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond.

76. The method according to claim 75, wherein R₁₃ is selected from the group consisting of 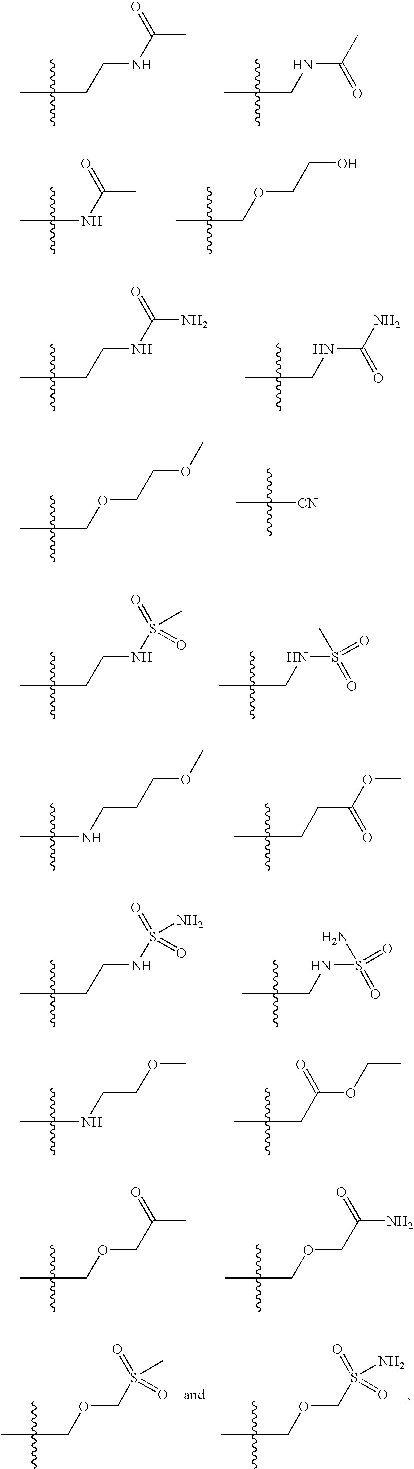
each substituted or unsubstituted.

77. The method according to claim 75 further comprising
reacting the compound having the formula 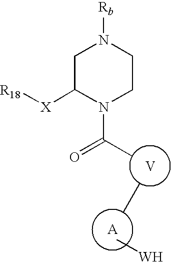
with a cyclic halide of the formula 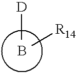
under conditions that form a compound having the formula 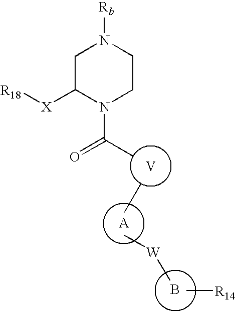
wherein
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
D is selected from the group consisting of —F, —Cl and —Br; and
R₁₄ is selected from the group consisting of 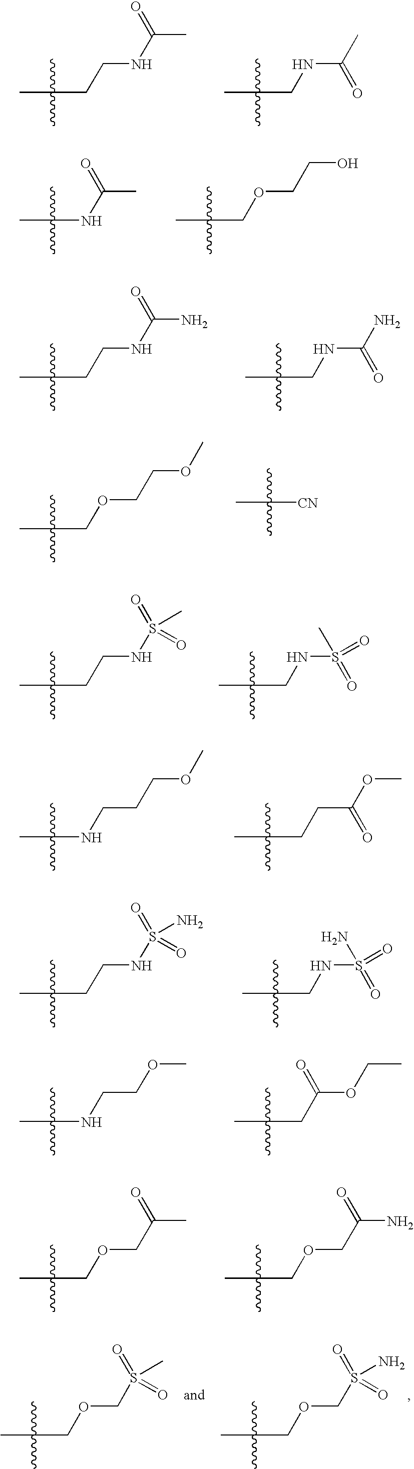
each substituted or unsubstituted.

78. The method of claim 77 where R₁₄ is —CN, the method further comprising:
converting the —CN group under conditions that forms an acyl-derivative having the formula 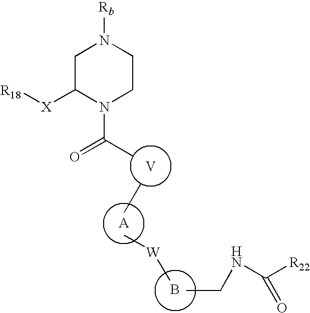
where
R₂₂ is selected from the group containing C₁₋₁₀alkyl and amino, substituted or unsubstituted.

79. The method according to claim 74, wherein X is selected from the group consisting of —CH₂— and —CH₂CH₂—.

80. The method according to claim 74, wherein R₁₈ is an unsubstituted phenyl

81. The method according to claim 74, wherein R₁₈ is a substituted phenyl and the substituent is selected from the group consisting of 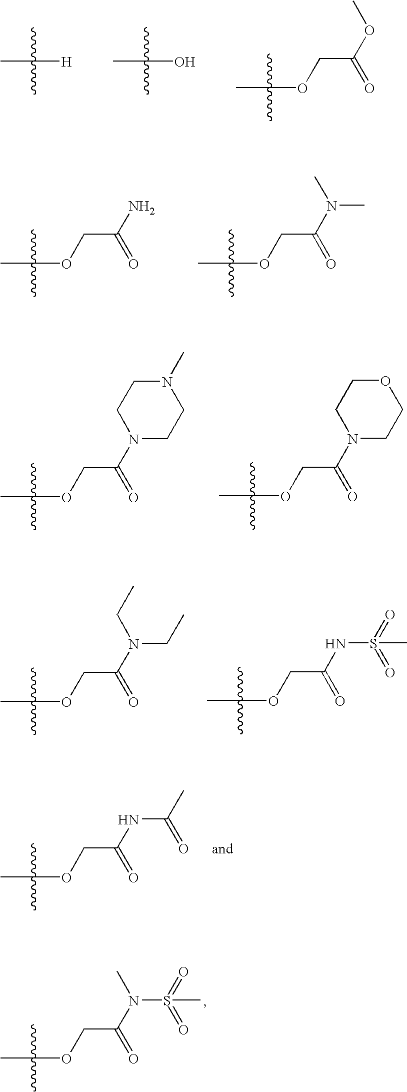
each substituted or unsubstituted.

82. The method according to claim 74, wherein ring V is selected from the group consisting of: 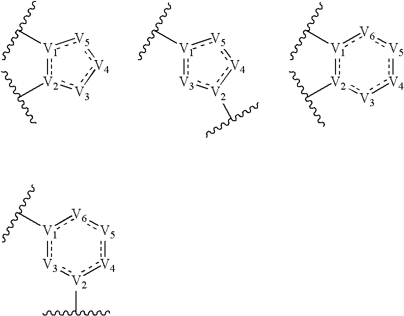
where
V₁ and V₂ are each independently selected from the group consisting of —CR₂₂— and —N—;
V₃ is selected from the group consisting of —CH₂—, —CH═, —NH—, —N═, —O— and —S—;
V₄, V₅ and V₆ are each independently selected from the group consisting of —CR₁₉R₂₀—, —NR₂₁—, —O— and —S—, and at least one of V₅ and V₆ is selected from the group consisting of —CH₂—, —CH═, —NH—, —N═, —O— and —S—;
R₁₉, R₂₀ and R₂₂ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₂₀ and R₂₂ are each independently absent when the carbon atom to which it is bound forms part of a double bond; and
R₂₁ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₂₁ is absent when the atom to which it is bound forms part of a double bond;
or any two R₁₉, R₂₀ and R₂₁ are taken together to form a 3, 4, 5, 6 or 7 membered ring.

83. The method according to claim 75, wherein ring A is selected from the group consisting of (C₃₋₇)cycloalkyl, hetero(C₃₋₇)cycloalkyl, (C₄₋₇)aryl and hetero(C₁₋₆)aryl, each substituted or unsubstituted.

84. The method according to claim 75, wherein ring A is selected from the group consisting of phenyl and hetero(C₁₋₅)aryl, each substituted or unsubstituted.

85. The method according to claim 75, wherein ring A is substituted with a substituent selected from the group consisting of 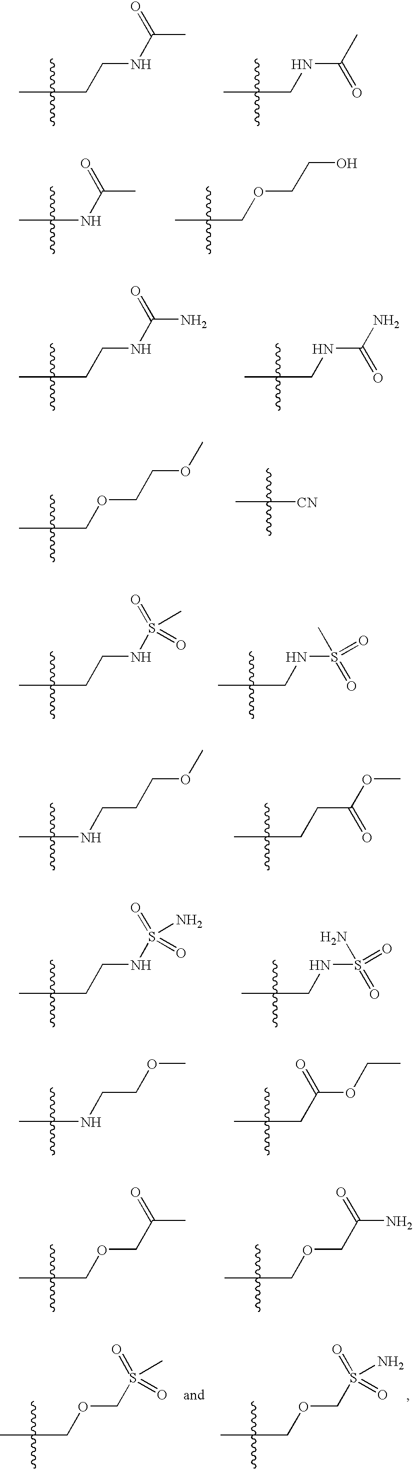
each substituted or unsubstituted.

86. The method according to claim 77, wherein ring B is selected from the group consisting of phenyl and hetero(C₁₋₁₀)aryl, each substituted or unsubstituted.

87. The method according to claim 77, wherein ring B is substituted with a substituent selected from the group consisting of 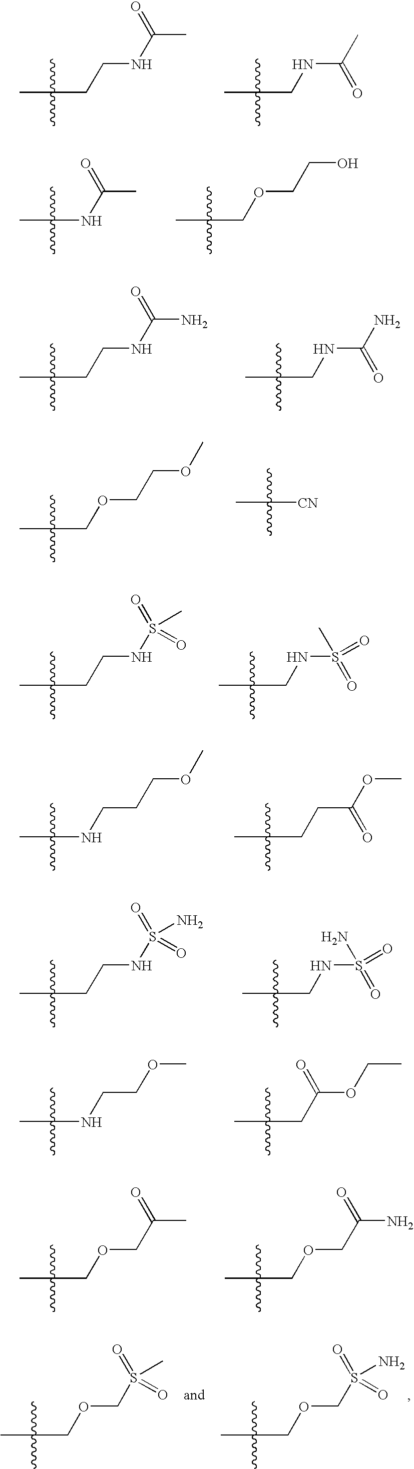
each substituted or unsubstituted.

88. The method according to claim 74 further comprising:
coupling a compound of the formula 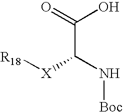
to another compound of the formula 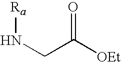
under conditions that form a compound having the formula 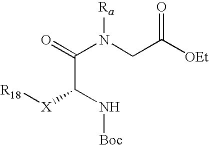
cyclizing the compound immediately above to form a compound having the formula 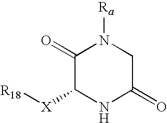
reducing the compound immediately above to a product having the formula 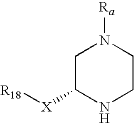
wherein
Rₐ is R₁ or benzyl.

89. A compound of the formula 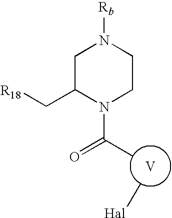
wherein
ring V is a 3, 5, or 6 membered, saturated, unsaturated or aromatic carbocycle or heterocycle, each substituted or unsubstituted;
Hal is selected from the group consisting of —Br, —I, —OTf, and —OMs;
R_{b} is selected from the group consisting of R₁, benzyl and a Boc group, where R₁ is selected from the group consisting of hydrogen and a substituent convertible in vivo to hydrogen; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

90. The compound according to claim 89, wherein R₁₈ is a unsubstituted phenyl,

91. The compound according to claim 89, wherein R₁₈ is a substituted phenyl, wherein the substituent is selected from the group consisting of 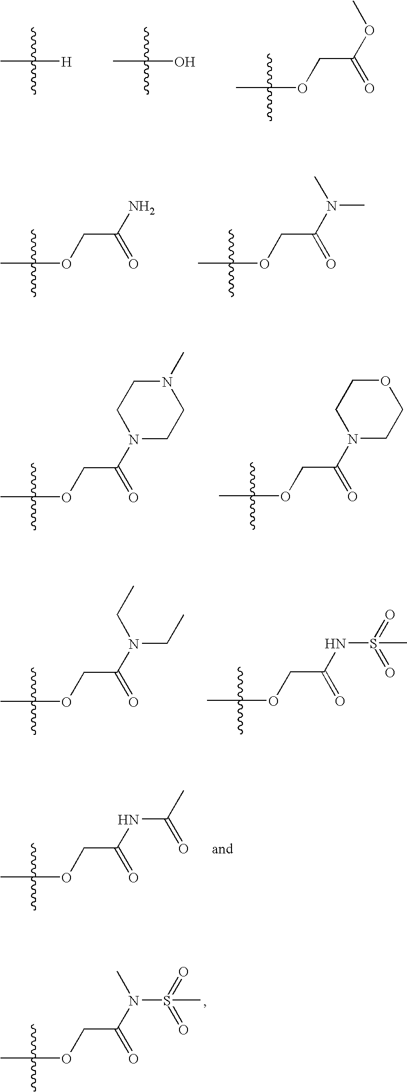
each substituted or unsubstituted.

92. The compound according to claim 89, wherein R₁ is hydrogen, methyl or ethyl.

93. A compound of the formula 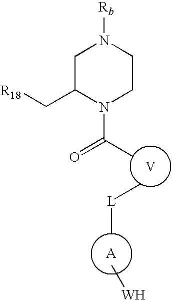
wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring V is a 3, 5, or 6, membered, saturated, unsaturated or aromatic carbocycle or heterocycle, each substituted or unsubstituted;
L is absent or is a linker providing 1, 2 or 3 atom separation between ring V and ring A, wherein the atoms of the linker providing the separation are selected from the group consisting of carbon, oxygen, nitrogen, and sulfur atoms;
W is absent or is selected from the group consisting of —CR₁₅R₁₆—, —NR₁₇—, —O— and —S—;
R_{b} is selected from the group consisting of R₁, benzyl and a Boc group, where R₁ is selected from the group consisting of hydrogen and a substituent convertible in vivo to hydrogen;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

94. The compound according to claim 93, wherein L is absent.

95. The compound according to claim 93, wherein R₁ is hydrogen, methyl or ethyl.

96. The compound according to claim 93, wherein R₁₈ is a unsubstituted phenyl,

97. The compound according to claim 93, wherein R₁₈ is a substituted phenyl, wherein the substituent is selected from the group consisting of 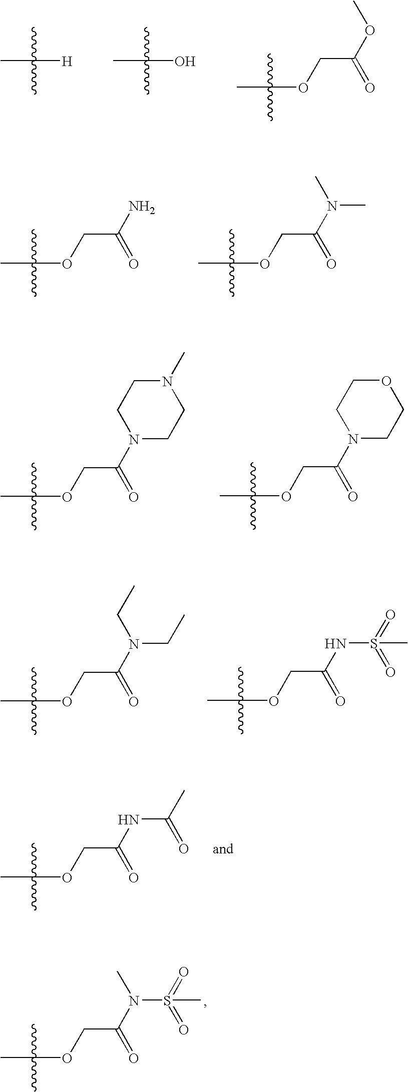
each substituted or unsubstituted.

98. A compound of the formula 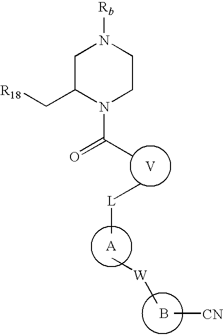
wherein
ring A is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring B is selected from the group consisting of (C₃₋₇)cycloalkyl, hetero(C₃₋₇)cycloalkyl, (C₉₋₁₂)bicycloalkyl, hetero (C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted;
ring V is a 3, 5, or 6 membered, saturated, unsaturated or aromatic carbocycle or heterocycle, each substituted or unsubstituted;
L is absent or is a linker providing 1, 2 or 3 atom separation between ring V and ring A, wherein the atoms of the linker providing the separation are selected from the group consisting of carbon, oxygen, nitrogen, and sulfur atoms;
W is absent or is selected from the group consisting of —CR₁₅R₁₆—, —NR₁₇—, —O— and —S—;
Y is selected from the group consisting of —C(O)— and —S(O)₂—;
R_{b} is selected from the group consisting of R₁, benzyl and a Boc group;
R₁ is selected from the group consisting of hydrogen and a substituent convertible in vivo to hydrogen;
R₁₅ and R₁₆ are each independently selected from the group consisting of hydrogen, halo, nitro, cyano, thio, oxy, hydroxy, carbonyloxy, alkoxy, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₇)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted, or R₁₅ and R₁₆ are taken together to form oxo, or R₁₆ is absent when the atom to which it is bound forms part of a double bond;
R₁₇ is selected from the group consisting of hydrogen, cyano, thio, oxy, hydroxy, carbonyloxy, alkoxy, aryloxy, heteroaryloxy, carbonyl, oxycarbonyl, aminocarbonyl, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₄₋₇)aryl and hetero(C₁₋₁₀)aryl, each substituted or unsubstituted, or R₁₇ is absent when the atom to which it is bound forms part of a double bond; and
R₁₈ is selected from the group consisting of hydrogen, halo, nitro, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, (C₃₋₇)cycloalkyl(C₁₋₅)alkyl, hetero(C₃₋₇)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₇)cycloalkyl, hetero(C₃₋₇)cycloalkyl, (C₉₋₁₂)bicycloalkyl, hetero(C₃₋₁₂)bicycloalkyl, (C₄₋₇)aryl, hetero(C₁₋₁₀)aryl, (C₉₋₁₂)bicycloaryl and hetero(C₄₋₁₂)bicycloaryl, each substituted or unsubstituted.

99. The compound according to claim 98, wherein R₁₈ is unsubstituted phenyl.

100. The compound according to claim 98, wherein R₁₈ is a substituted phenyl, wherein the substituent is selected from the group consisting of 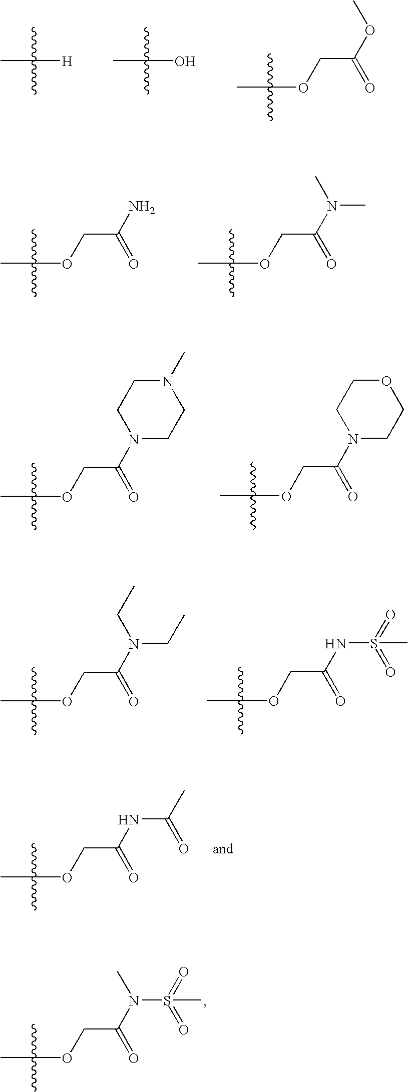
each substituted or unsubstituted.

101. The compound according to claim 98, wherein L is absent.

102. The compound according to claim 98, wherein R₁ is hydrogen, methyl or ethyl.
